SEQUENCE LISTING (57 sequences)

SEQ ID NO: 1 - RNA, 20 nt, Homo sapiens
  source 1..20: mol_type = mRNA
gctccgcgctgctcaacccc

SEQ ID NO: 2 - RNA, 20 nt, Homo sapiens
  source 1..20: mol_type = mRNA
agcctcccgaaccagagcga

SEQ ID NO: 3 - RNA, 20 nt, Homo sapiens
  source 1..20: mol_type = mRNA
cacagagccatcaaaatcac

SEQ ID NO: 4 - RNA, 20 nt, Homo sapiens
  source 1..20: mol_type = mRNA
cagagccatcaaaatcacag

SEQ ID NO: 5 - RNA, 20 nt, Homo sapiens
  source 1..20: mol_type = mRNA
agagccatcaaaatcacagt

SEQ ID NO: 6 - RNA, 20 nt, Homo sapiens
  source 1..20: mol_type = mRNA
gagccatcaaaatcacagtg

SEQ ID NO: 7 - RNA, 20 nt, Homo sapiens
  source 1..20: mol_type = mRNA
agccatcaaaatcacagtgg

SEQ ID NO: 8 - RNA, 20 nt, Homo sapiens
  source 1..20: mol_type = mRNA
gccatcaaaatcacagtgga

SEQ ID NO: 9 - RNA, 20 nt, Homo sapiens
  source 1..20: mol_type = mRNA
agcttcactctgaccatcac

SEQ ID NO: 10 - RNA, 20 nt, Homo sapiens
  source 1..20: mol_type = mRNA
ccagatggcactctggtcac

SEQ ID NO: 11 - RNA, 20 nt, Homo sapiens
  source 1..20: mol_type = mRNA
ggcaatgatgaaaactactc

SEQ ID NO: 12 - RNA, 20 nt, Homo sapiens
  source 1..20: mol_type = mRNA
acaagaccctgcccatcgct

SEQ ID NO: 13 - RNA, 20 nt, Homo sapiens
  source 1..20: mol_type = mRNA
tagagccatcaaaatcacag

SEQ ID NO: 14 - RNA, 20 nt, Homo sapiens
  source 1..20: mol_type = mRNA
ccggatggcactctggtcac

SEQ ID NO: 15 - RNA, 20 nt, Homo sapiens
  source 1..20: mol_type = mRNA
gatggcactctggtcactgt

SEQ ID NO: 16 - RNA, 20 nt, Homo sapiens
  source 1..20: mol_type = mRNA
caatgatgaaaactactcgg

SEQ ID NO: 17 - RNA, 20 nt, Homo sapiens
  source 1..20: mol_type = mRNA
gcagccatgaagaaccaggt

SEQ ID NO: 18 - RNA, 20 nt, Homo sapiens
  source 1..20: mol_type = mRNA
tcactctgaccatcactgtc

SEQ ID NO: 19 - RNA, 20 nt, Homo sapiens
  source 1..20: mol_type = mRNA
gatggcactctggtcaccgt

SEQ ID NO: 20 - DNA, 20 bp, synthetic construct
  modified_base 10: mod_base = OTHER; note = 5-methylcytosine
  modified_base 13: mod_base = OTHER; note = 5-methylcytosine
  modified_base 15: mod_base = OTHER; note = 5-methylcytosine
  modified_base 20: mod_base = OTHER; note = 5-methylcytosine
  source 1..20: mol_type = other DNA
ggggttgagnagngnggagn

SEQ ID NO: 21 - DNA, 20 bp, synthetic construct
  modified_base 2: mod_base = OTHER; note = 5-methylcytosine
  modified_base 4: mod_base = OTHER; note = 5-methylcytosine
  modified_base 6: mod_base = OTHER; note = 5-methylcytosine
  modified_base 12: mod_base = OTHER; note = 5-methylcytosine
  modified_base 19: mod_base = OTHER; note = 5-methylcytosine
  source 1..20: mol_type = other DNA
tngntntggttngggaggnt

SEQ ID NO: 22 - DNA, 20 bp, synthetic construct
  modified_base 14: mod_base = OTHER; note = 5-methylcytosine
  modified_base 16: mod_base = OTHER; note = 5-methylcytosine
  source 1..20: mol_type = other DNA
gtgattttgatggntntgtg

SEQ ID NO: 23 - DNA, 20 bp, synthetic construct
  modified_base 1: mod_base = OTHER; note = 5-methylcytosine
  modified_base 16: mod_base = OTHER; note = 5-methylcytosine
  modified_base 18: mod_base = OTHER; note = 5-methylcytosine
  source 1..20: mol_type = other DNA
ntgtgattttgatggntntg

SEQ ID NO: 24 - DNA, 20 bp, synthetic construct
  modified_base 2: mod_base = OTHER; note = 5-methylcytosine
  modified_base 17: mod_base = OTHER; note = 5-methylcytosine
  modified_base 19: mod_base = OTHER; note = 5-methylcytosine
  source 1..20: mol_type = other DNA
antgtgattttgatggntnt

SEQ ID NO: 25 - DNA, 20 bp, synthetic construct
  modified_base 1: mod_base = OTHER; note = 5-methylcytosine
  modified_base 3: mod_base = OTHER; note = 5-methylcytosine
  modified_base 18: mod_base = OTHER; note = 5-methylcytosine
  modified_base 20: mod_base = OTHER; note = 5-methylcytosine
  source 1..20: mol_type = other DNA
nantgtgattttgatggntn

SEQ ID NO: 26 - DNA, 20 bp, Synthetic construct
  modified_base 1: mod_base = OTHER; note = 5-methylcytosine
  modified_base 2: mod_base = OTHER; note = 5-methylcytosine
  modified_base 4: mod_base = OTHER; note = 5-methylcytosine
  modified_base 19: mod_base = OTHER; note = 5-methylcytosine
  source 1..20: mol_type = other DNA
nnantgtgattttgatggnt

SEQ ID NO: 27 - DNA, 20 bp, synthetic construct
  modified_base 2: mod_base = OTHER; note = 5-methylcytosine
  modified_base 3: mod_base = OTHER; note = 5-methylcytosine
  modified_base 5: mod_base = OTHER; note = 5-methylcytosine
  modified_base 20: mod_base = OTHER; note = 5-methylcytosine
  source 1..20: mol_type = other DNA
tnnantgtgattttgatggn

SEQ ID NO: 28 - DNA, 20 bp, synthetic construct
  modified_base 9: mod_base = OTHER; note = 5-methylcytosine
  modified_base 19: mod_base = OTHER; note = 5-methylcytosine
  source 1..20: mol_type = other DNA
gtgatggtnagagtgaagnt

SEQ ID NO: 29 - DNA, 20 bp, synthetic construct
  modified_base 5: mod_base = OTHER; note = 5-methylcytosine
  modified_base 6: mod_base = OTHER; note = 5-methylcytosine
  modified_base 13: mod_base = OTHER; note = 5-methylcytosine
  modified_base 14: mod_base = OTHER; note = 5-methylcytosine
  modified_base 17: mod_base = OTHER; note = 5-methylcytosine
  source 1..20: mol_type = other DNA
gtgannagagtgnnatntgg

SEQ ID NO: 30 - DNA, 20 bp, synthetic construct
  modified_base 11: mod_base = OTHER; note = 5-methylcytosine
  modified_base 14: mod_base = OTHER; note = 5-methylcytosine
  modified_base 19: mod_base = OTHER; note = 5-methylcytosine
  modified_base 20: mod_base = OTHER; note = 5-methylcytosine
  source 1..20: mol_type = other DNA
gagtagttttnatnattgnn

SEQ ID NO: 31 - DNA, 20 bp, synthetic construct
  modified_base 3: mod_base = OTHER; note = 5-methylcytosine
  modified_base 10: mod_base = OTHER; note = 5-methylcytosine
  modified_base 16: mod_base = OTHER; note = 5-methylcytosine
  source 1..20: mol_type = other DNA
agngatgggnagggtnttgt

SEQ ID NO: 32 - DNA, 20 bp, synthetic construct
  modified_base 1: mod_base = OTHER; note = 5-methylcytosine
  modified_base 16: mod_base = OTHER; note = 5-methylcytosine
  modified_base 18: mod_base = OTHER; note = 5-methylcytosine
  source 1..20: mol_type = other DNA
ntgtgattttgatggntnta

SEQ ID NO: 33 - DNA, 20 bp, synthetic construct
  modified_base 5: mod_base = OTHER; note = 5-methylcytosine
  modified_base 6: mod_base = OTHER; note = 5-methylcytosine
  modified_base 13: mod_base = OTHER; note = 5-methylcytosine
  modified_base 14: mod_base = OTHER; note = 5-methylcytosine
  modified_base 17: mod_base = OTHER; note = 5-methylcytosine
  modified_base 18: mod_base = OTHER; note = 5-methylcytosine
  source 1..20: mol_type = other DNA
gtgannagagtgnnatnngg

SEQ ID NO: 34 - DNA, 20 bp, synthetic construct
  modified_base 2: mod_base = OTHER; note = 5-methylcytosine
  modified_base 8: mod_base = OTHER; note = 5-methylcytosine
  modified_base 9: mod_base = OTHER; note = 5-methylcytosine
  modified_base 16: mod_base = OTHER; note = 5-methylcytosine
  modified_base 17: mod_base = OTHER; note = 5-methylcytosine
  modified_base 20: mod_base = OTHER; note = 5-methylcytosine
  source 1..20: mol_type = other DNA
anagtgannagagtgnnatn

SEQ ID NO: 35 - DNA, 20 bp, synthetic construct
  modified_base 1: mod_base = OTHER; note = 5-methylcytosine
  modified_base 2: mod_base = OTHER; note = 5-methylcytosine
  modified_base 13: mod_base = OTHER; note = 5-methylcytosine
  modified_base 16: mod_base = OTHER; note = 5-methylcytosine
  source 1..20: mol_type = other DNA
nngagtagttttnatnattg

SEQ ID NO: 36 - DNA, 20 bp, synthetic construct
  modified_base 2: mod_base = OTHER; note = 5-methylcytosine
  modified_base 3: mod_base = OTHER; note = 5-methylcytosine
  modified_base 9: mod_base = OTHER; note = 5-methylcytosine
  modified_base 12: mod_base = OTHER; note = 5-methylcytosine
  modified_base 17: mod_base = OTHER; note = 5-methylcytosine
  modified_base 20: mod_base = OTHER; note = 5-methylcytosine
  source 1..20: mol_type = other DNA
anntggttnttnatggntgn

SEQ ID NO: 37 - DNA, 20 bp, synthetic construct
  modified_base 3: mod_base = OTHER; note = 5-methylcytosine
  modified_base 13: mod_base = OTHER; note = 5-methylcytosine
  source 1..20: mol_type = other DNA
ganagtgatggtnagagtga

SEQ ID NO: 38 - DNA, 20 bp, synthetic construct
  modified_base 2: mod_base = OTHER; note = 5-methylcytosine
  modified_base 8: mod_base = OTHER; note = 5-methylcytosine
  modified_base 9: mod_base = OTHER; note = 5-methylcytosine
  modified_base 16: mod_base = OTHER; note = 5-methylcytosine
  modified_base 17: mod_base = OTHER; note = 5-methylcytosine
  modified_base 20: mod_base = OTHER; note = 5-methylcytosine
  source 1..20: mol_type = other DNA
anggtgannagagtgnnatn

SEQ ID NO: 39 - DNA, 20 bp, Homo sapiens
  source 1..20: mol_type = genomic DNA
gctccgcgctgctcaacccc

SEQ ID NO: 40 - DNA, 20 bp, Homo sapiens
  source 1..20: mol_type = genomic DNA
agcctcccgaaccagagcga

SEQ ID NO: 41 - DNA, 20 bp, Homo sapiens
  source 1..20: mol_type = genomic DNA
cacagagccatcaaaatcac

SEQ ID NO: 42 - DNA, 20 bp, Homo sapiens
  source 1..20: mol_type = genomic DNA
cagagccatcaaaatcacag

SEQ ID NO: 43 - DNA, 20 bp, Homo sapiens
  source 1..20: mol_type = genomic DNA
agagccatcaaaatcacagt

SEQ ID NO: 44 - DNA, 20 bp, Homo sapiens
  source 1..20: mol_type = genomic DNA
gagccatcaaaatcacagtg

SEQ ID NO: 45 - DNA, 20 bp, Homo sapiens
  source 1..20: mol_type = genomic DNA
agccatcaaaatcacagtgg

SEQ ID NO: 46 - DNA, 20 bp, Homo sapiens
  source 1..20: mol_type = genomic DNA
gccatcaaaatcacagtgga

SEQ ID NO: 47 - DNA, 20 bp, Homo sapiens
  source 1..20: mol_type = genomic DNA
agcttcactctgaccatcac

SEQ ID NO: 48 - DNA, 20 bp, Homo sapiens
  source 1..20: mol_type = genomic DNA
ccagatggcactctggtcac

SEQ ID NO: 49 - DNA, 20 bp, Homo sapiens
  source 1..20: mol_type = genomic DNA
ggcaatgatgaaaactactc

SEQ ID NO: 50 - DNA, 20 bp, Homo sapiens
  source 1..20: mol_type = genomic DNA
acaagaccctgcccatcgct

SEQ ID NO: 51 - DNA, 20 bp, Homo sapiens
  source 1..20: mol_type = genomic DNA
tagagccatcaaaatcacag

SEQ ID NO: 52 - DNA, 20 bp, Homo sapiens
  source 1..20: mol_type = genomic DNA
ccggatggcactctggtcac

SEQ ID NO: 53 - DNA, 20 bp, Homo sapiens
  source 1..20: mol_type = genomic DNA
gatggcactctggtcactgt

SEQ ID NO: 54 - DNA, 20 bp, Homo sapiens
  source 1..20: mol_type = genomic DNA
caatgatgaaaactactcgg

SEQ ID NO: 55 - DNA, 20 bp, Homo sapiens
  source 1..20: mol_type = genomic DNA
gcagccatgaagaaccaggt

SEQ ID NO: 56 - DNA, 20 bp, Homo sapiens
  source 1..20: mol_type = genomic DNA
tcactctgaccatcactgtc

SEQ ID NO: 57 - DNA, 20 bp, Homo sapiens
  source 1..20: mol_type = genomic DNA
gatggcactctggtcaccgt

(19) United States
(12) Patent Application Publication
(10) Pub. No.: US 2026/0124232 A1
(43) Pub. Date: May 7, 2026

(54) RUNX1 MODULATORS

(71) Applicant: Sanford Burnham Prebys Medical Discovery Institute, La Jolla, CA (US)

(72) Inventors: Carter PALMER, La Jolla, CA (US); Christine S. LIU, San Diego, CA (US); Linnea RANSOM, San Diego, CA (US); Nyssa WILLIAMS, Carlsbad, CA (US); Jerold CHUN, La Jolla, CA (US)

(21) Appl. No.: 19/326,037

(22) Filed: Sep. 11, 2025

Related U.S. Application Data
(63) Continuation of application No. PCT/US2024/019997, filed on Mar. 14, 2024.
(60) Provisional application No. 63/490,685, filed on Mar. 16, 2023.

(51) Int. Cl. A61K 31/7125; A61K 31/712; A61P 25/28; C12N 15/113

(52) U.S. Cl. CPC A61K 31/7125; A61K 31/712; A61P 25/28; C12N 15/113; C12N 2310/11; C12N 2310/315; C12N 2310/321; C12N 2310/3341; C12N 2310/341

(57) ABSTRACT

Provided herein are compounds, compositions, and methods for lowering expression levels of runt-related transcription factor 1 (RUNX1) in a cell, tissue or animal. Further provided are methods of improving memory and cognitive functioning in individuals with Down syndrome (DS) using an antisense compound targeted to a RUNX1 nucleic acid. Also provided are uses of disclosed compounds and compositions in the manufacture of a medicament for treatment of diseases and disorders. Further provided are methods of decreasing inflammation and slowing cognitive decline in individuals with dementia.

### CROSS REFERENCE

This application is a continuation of International Application No. PCT/US2024/019997, filed Mar. 14, 2024, and claims the benefit of U.S. Provisional Application No. 63/490,685, filed Mar. 16, 2023, all of which are incorporated by reference in their entirety herein.

### STATEMENT REGARDING FEDERALLY SPONSORED RESEARCH OR DEVELOPMENT

This invention was made with government support under R56 AG073965 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, generated on Mar. 13, 2024, is named 42256-615 301_SL.XML and is 92,074 bytes in size.

### BACKGROUND

RUNX1 is a transcription factor expressed in immune cells throughout the body. Within the central nervous system (CNS) it is expressed in microglia, the “resident immune cells” of the brain. Microglia have numerous functions and states. They are classically noted for modulating inflammation and are now recognized to have a multitude of diverse functions including synaptic maintenance, memory removal, CNS surveillance, aggregate formation, and others. It has been shown that individuals with Down syndrome (DS) have abnormal microglia, and that these are characterized by significant overexpression of RUNX1, a gene that lies on human chromosome 21. Additionally, it has been shown that RUNX1 overexpression is associated with the rare primary microgliopathy Nasu Hakola Disorder as well as microglia in Alzheimer's disease. RUNX1 expression in microglia increases with the development of Alzheimer's disease and has been identified to regulate disease pathways in microglia in neurodegeneration. Decreasing RUNX1 levels induces microglia turnover and appears to return microglia to a more homeostatic state, decreasing neuroinflammation as a result. There is currently a lack of effective therapeutics to decrease neuroinflammation in numerous neurodegenerative diseases, including Down syndrome associated dementia, Alzheimer's disease in the general population, and Nasu Hakola disorder.

### BRIEF SUMMARY OF THE INVENTION

Provided herein are methods to decrease RUNX1 levels to control microglial functional states. More specifically, the present application discloses the ability to decrease RUNX1 levels using antisense oligonucleotides (ASOs). Decreasing RUNX1 levels can modify transcriptomic, morphological, and functional aspects of microglia and may, for example, normalize the microglia in Down syndrome brains, Alzheimer's disease, and generally inflamed brains, in turn improving memory and potentially additional cognitive attributes for these individuals.

One embodiment provides an oligomeric compound or a salt thereof, comprising a modified oligonucleotide consisting of 10-30 linked nucleosides wherein the modified oligonucleotide has a nucleobase sequence that is at least 80% complementary to any of the nucleobase sequences of SEQ ID NO:1-19 when measured across the entire nucleobase sequence of the modified oligonucleotide, and wherein the modified oligonucleotide comprises at least one modification selected from a modified sugar, a sugar surrogate, and a modified internucleoside linkage.

One embodiment provides an oligomeric compound, or a salt thereof, comprising a modified oligonucleotide consisting of 10-30 linked nucleosides wherein the modified oligonucleotide has a nucleobase sequence that is targeted to any of the nucleobase sequences of SEQ ID NO: 39-57, when measured across the entire nucleobase sequence of the modified oligonucleotide, and wherein the modified oligonucleotide comprises at least one modification selected from a modified sugar, a sugar surrogate, and a modified internucleoside linkage.

One embodiment provides an oligomeric compound (formula ASO (1)) according to the following formula:

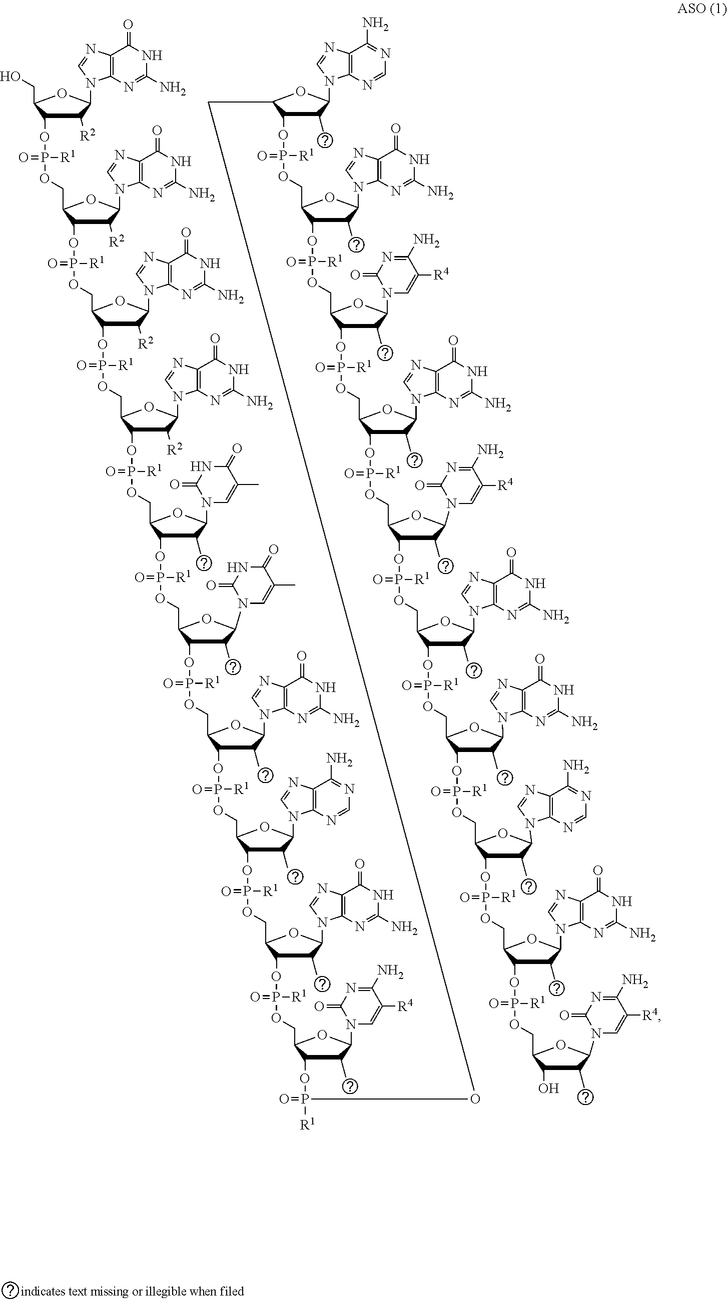

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (2)) according to the following formula:

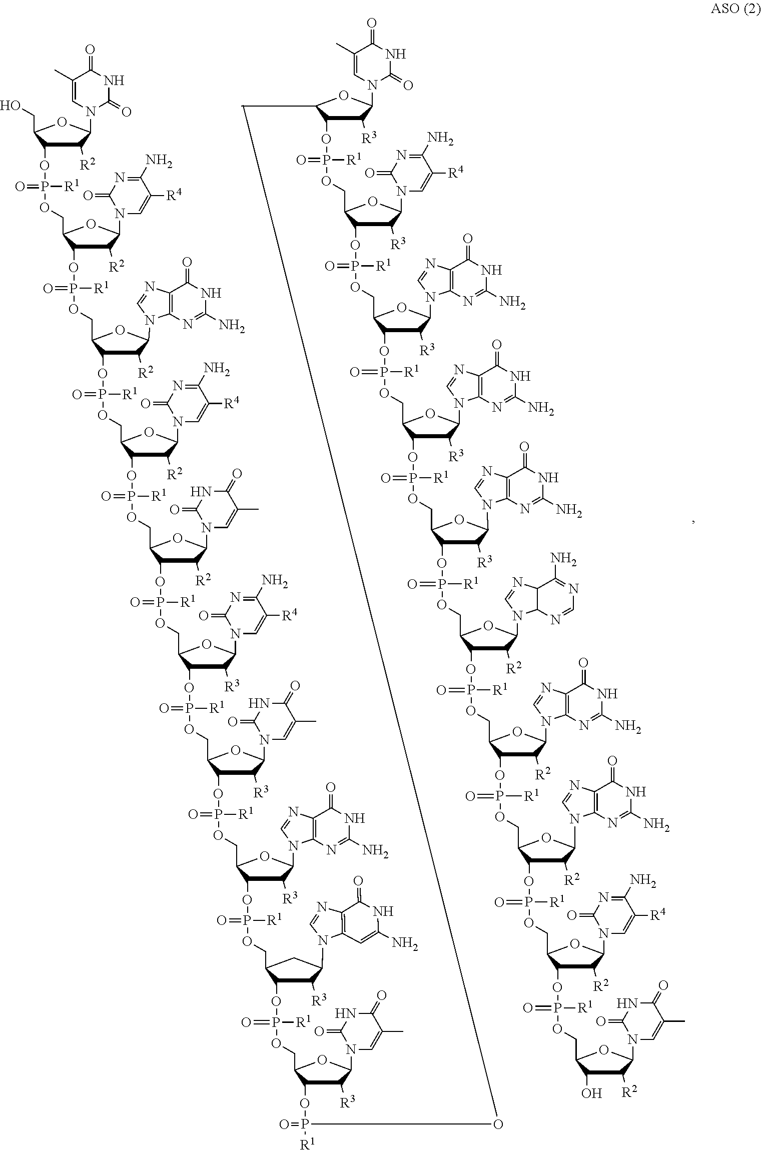

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OC₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (3)) according to the following formula:

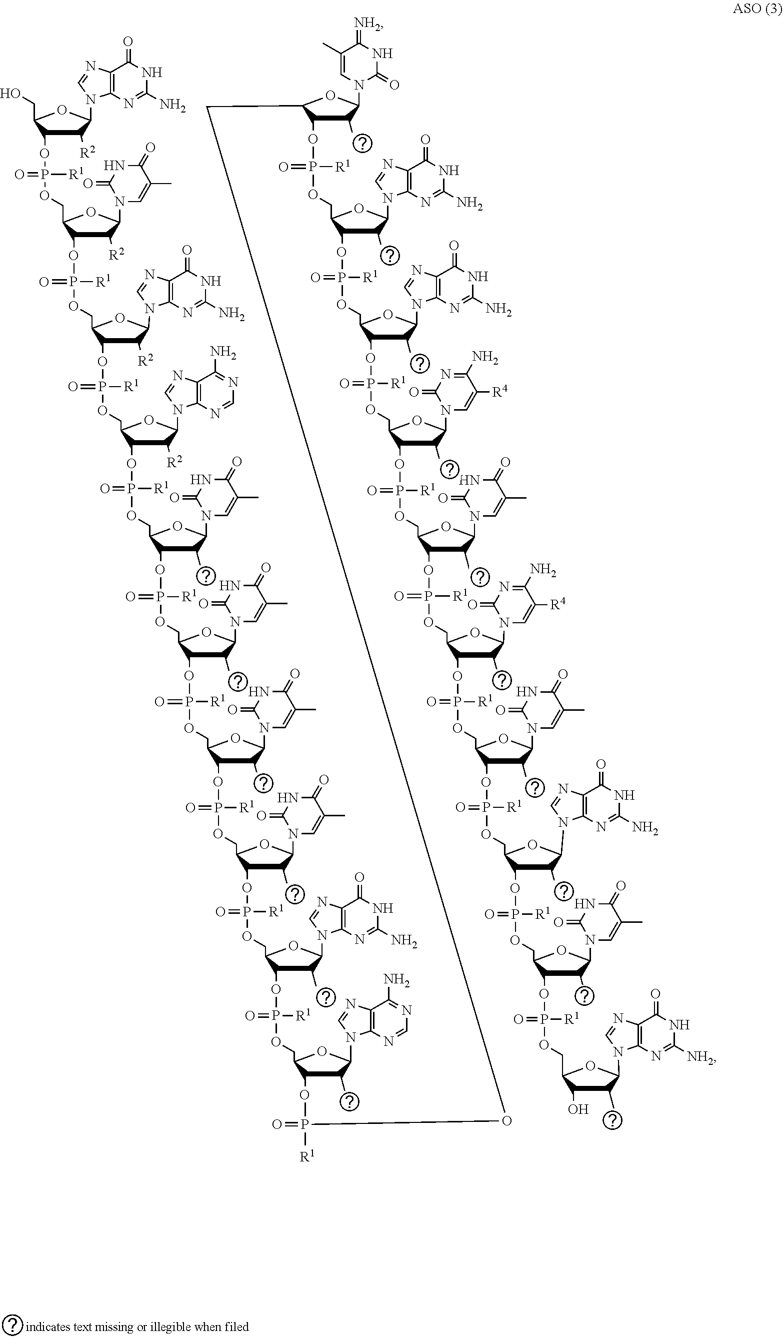

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃:
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (4)) according to the formula:

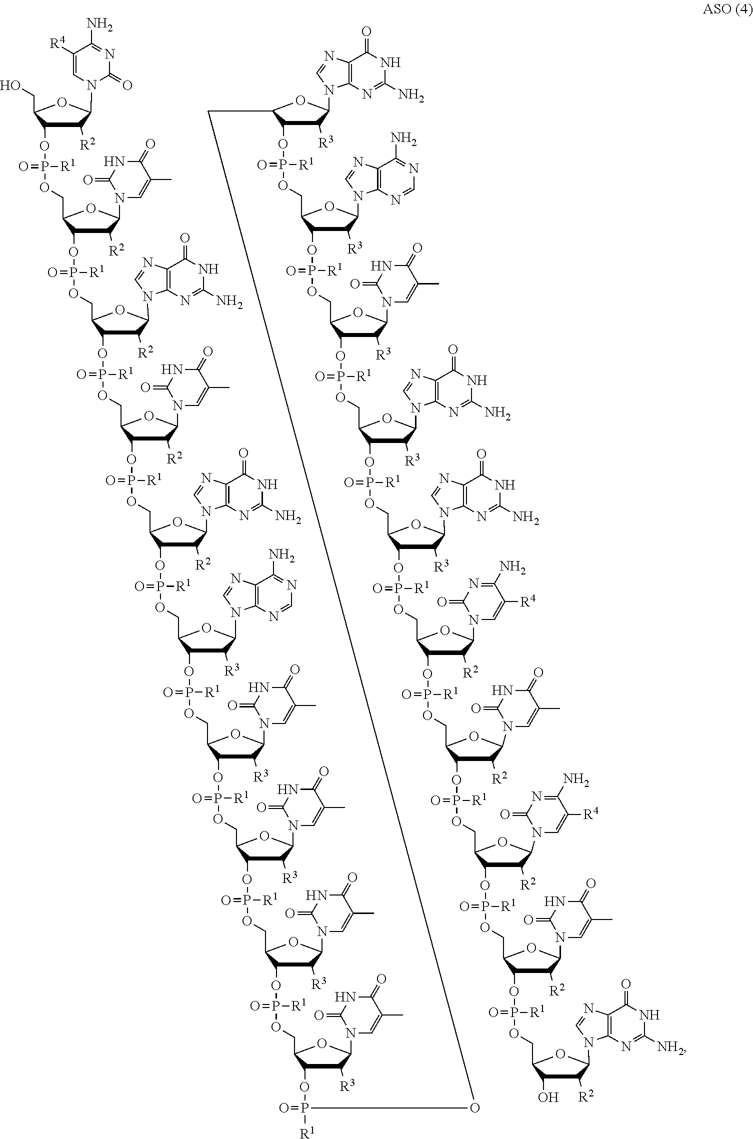

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (5)) according to the formula:

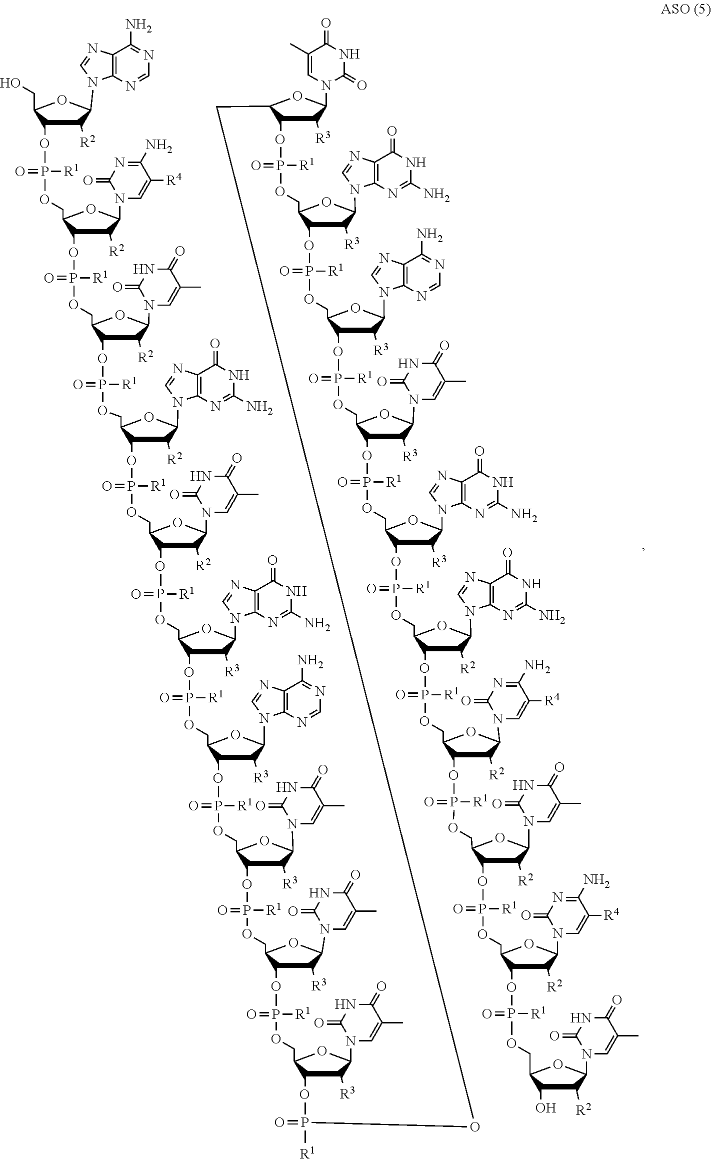

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (6)) according to the formula:

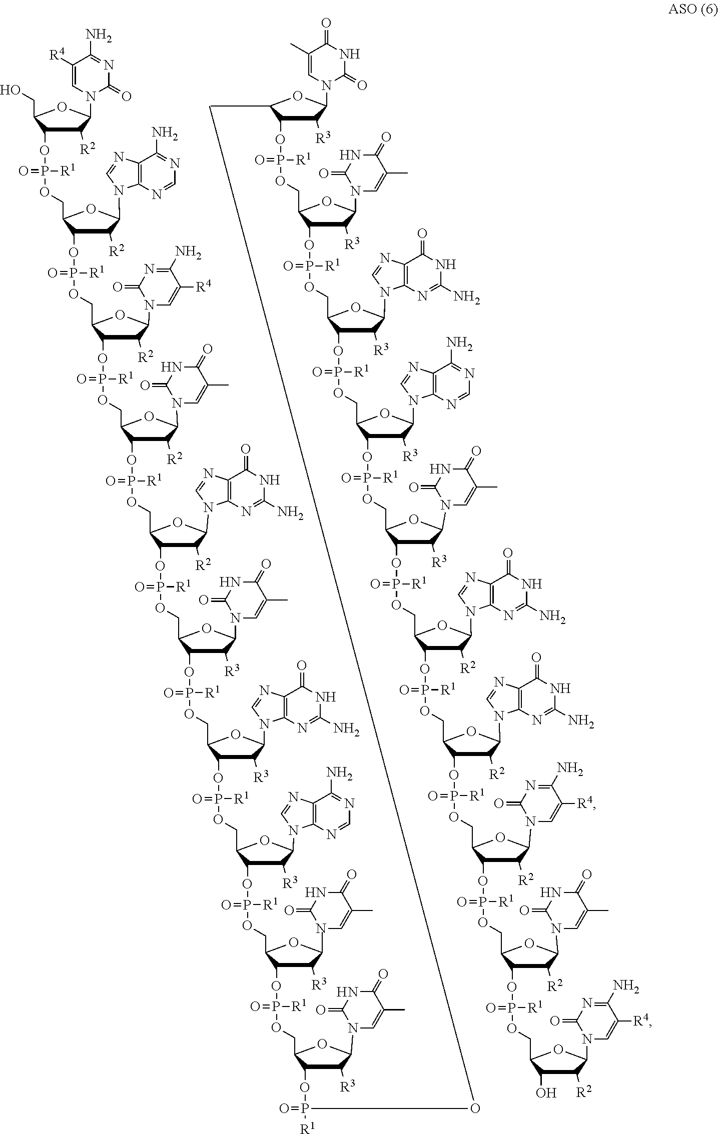

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides ani oligomeric compound (formula ASO (7)) according to the formula:

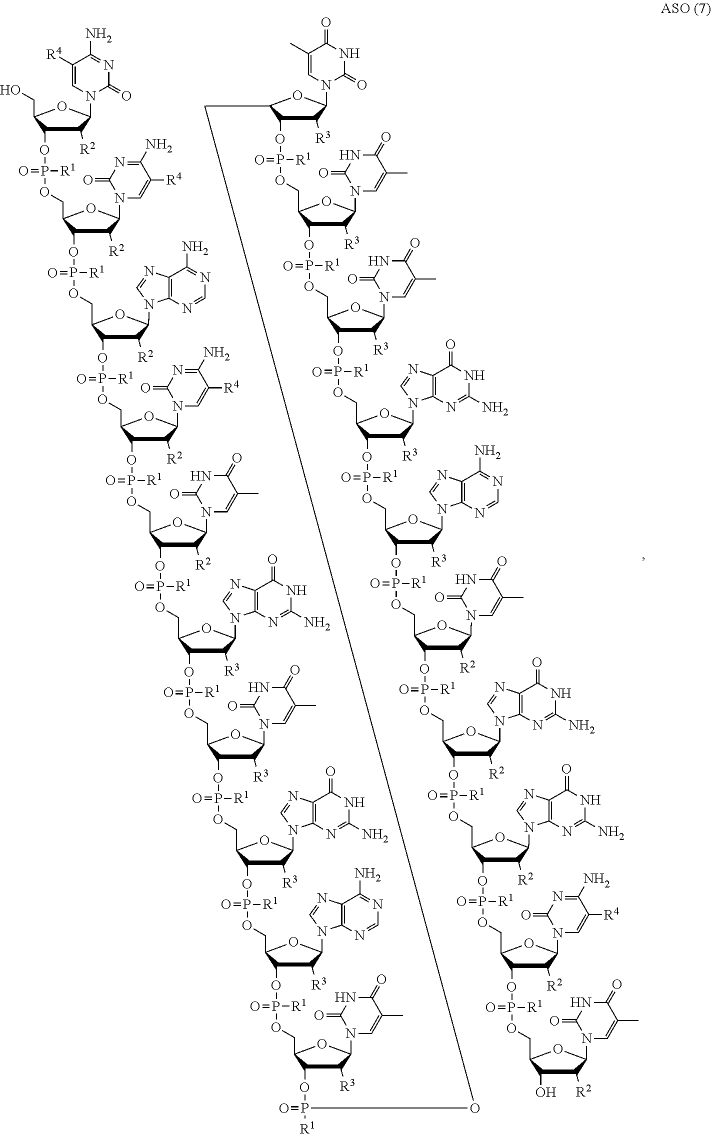

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (8)) according to the formula:

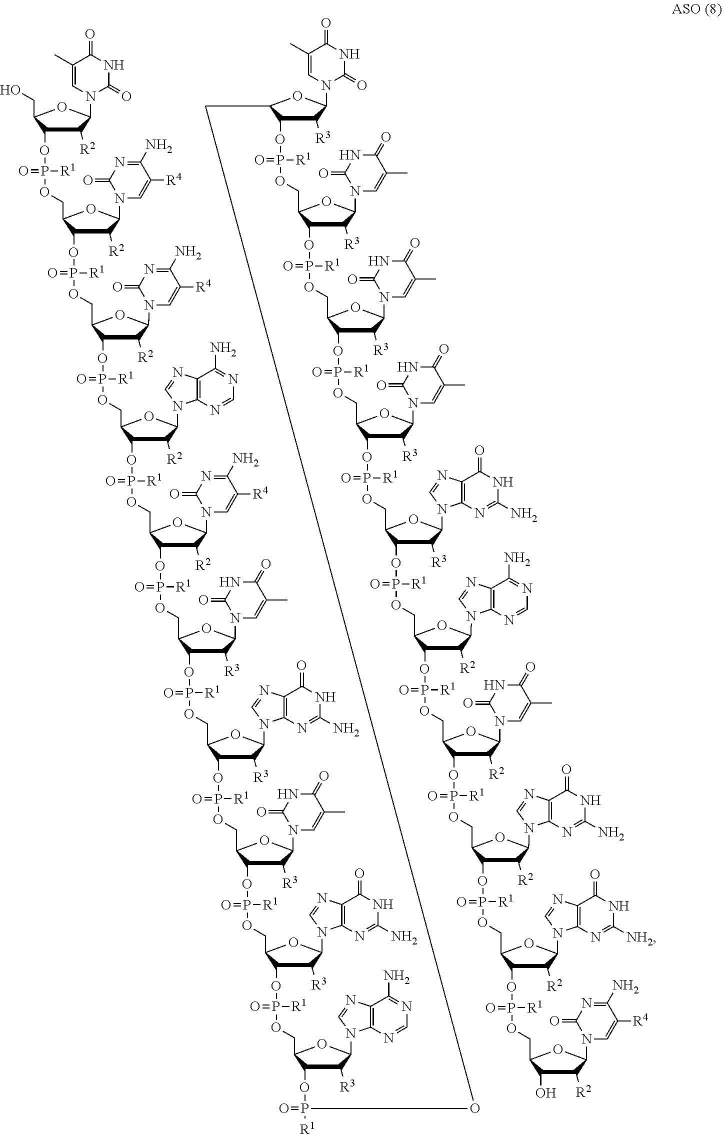

or a salt thereof, wherein:
each R¹ is independently selected from the group consisting of OH and SH;
each R² is independently selected from the group consisting of hydrogen, halo, OCH₃, OCF₃, OCH₂C-13, and OCH₂CH₂OCH₃, OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (9)) according to the formula:

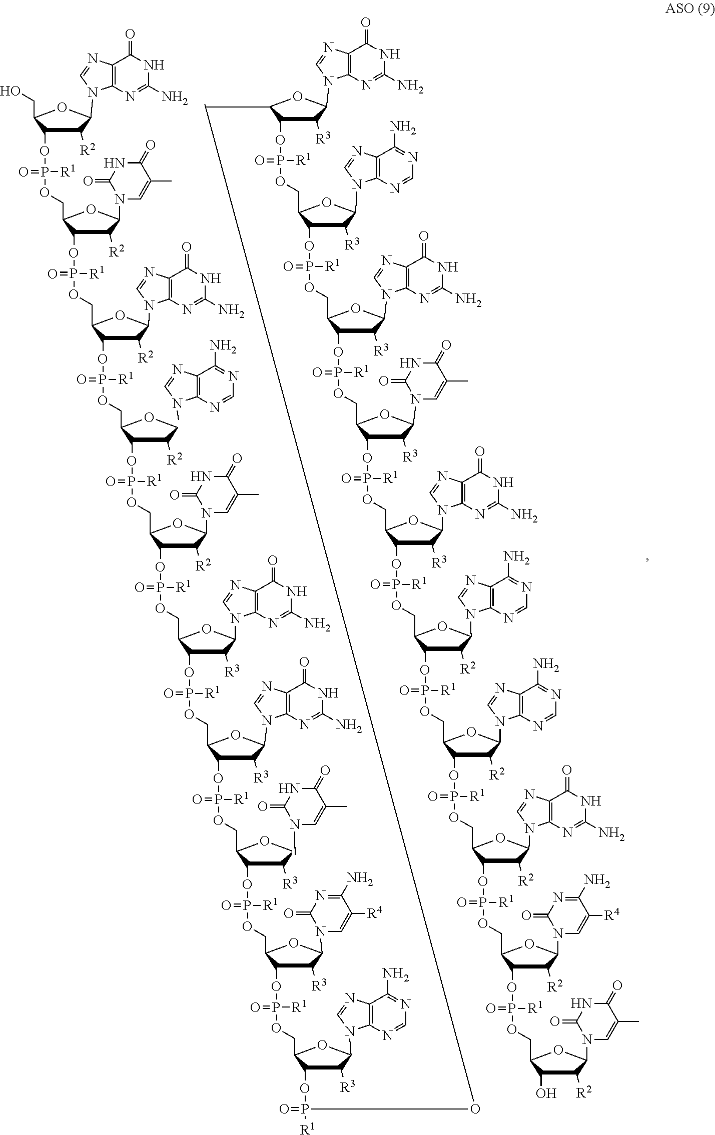

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (10)) according to the formula:

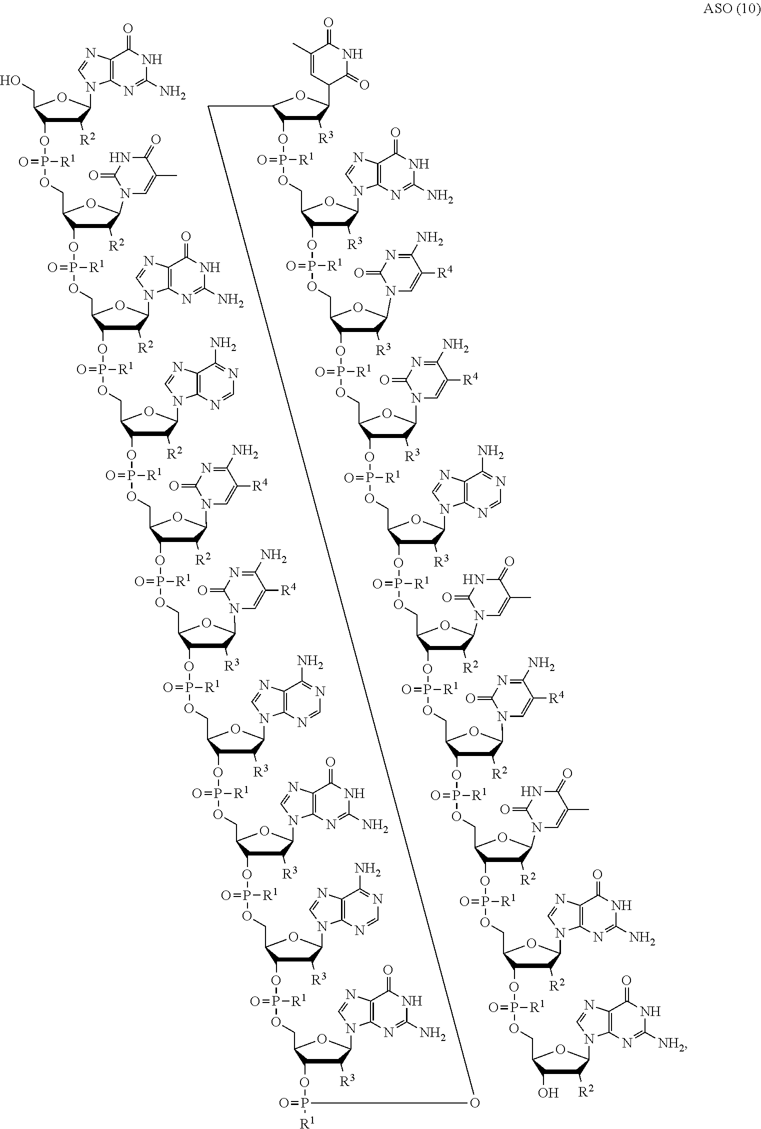

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (11)) according to the formula:

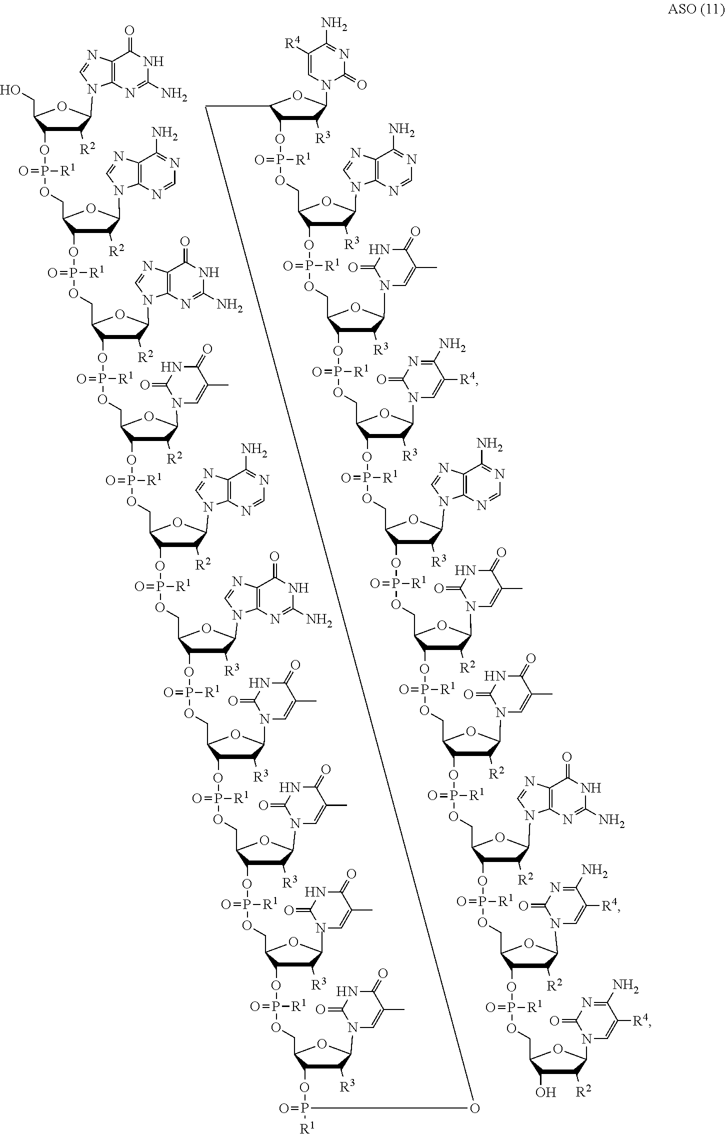

or a salt thereof, wherein:
each R¹ is independently selected from the group consisting of OH and SH;
each R² is independently selected from the group consisting of hydrogen, halo, OCH₃, OCF₃, OCH₂CH₃OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (12)) according to the formula:

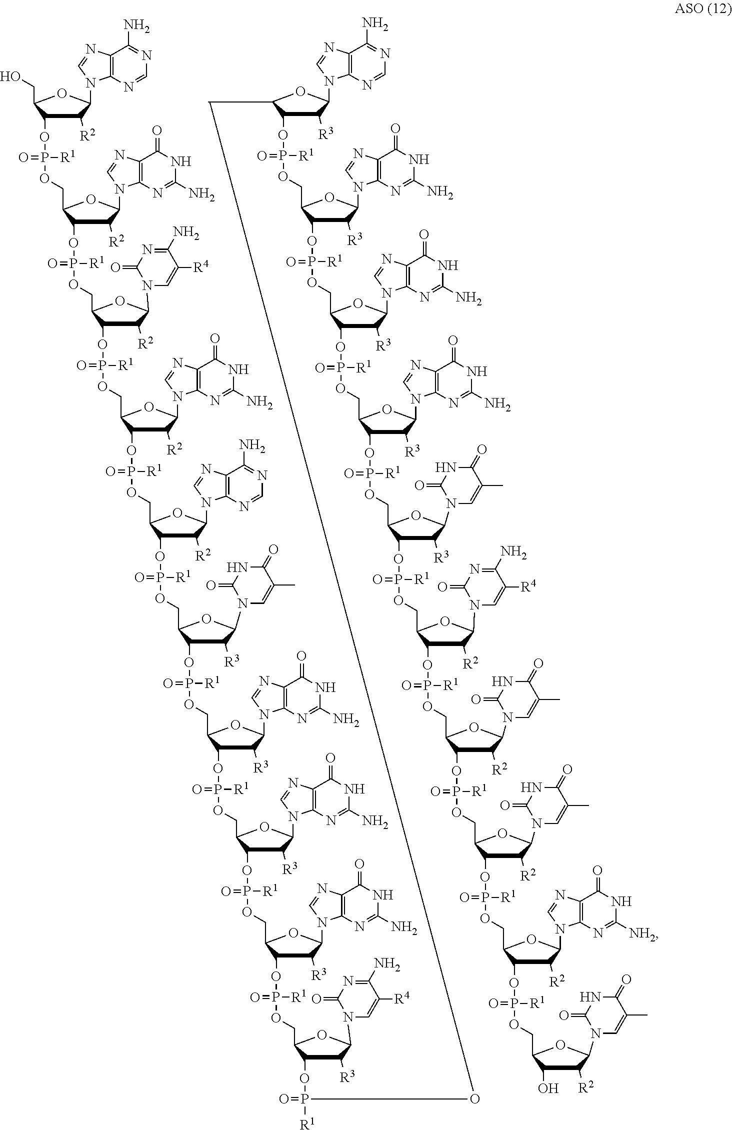

or a salt thereof, wherein.
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂F;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, PA is hydrogen. In some embodiments, R⁴ is CH₃.

One embodiment provides an oligomeric compound (formula ASO (13)) according to the formula:

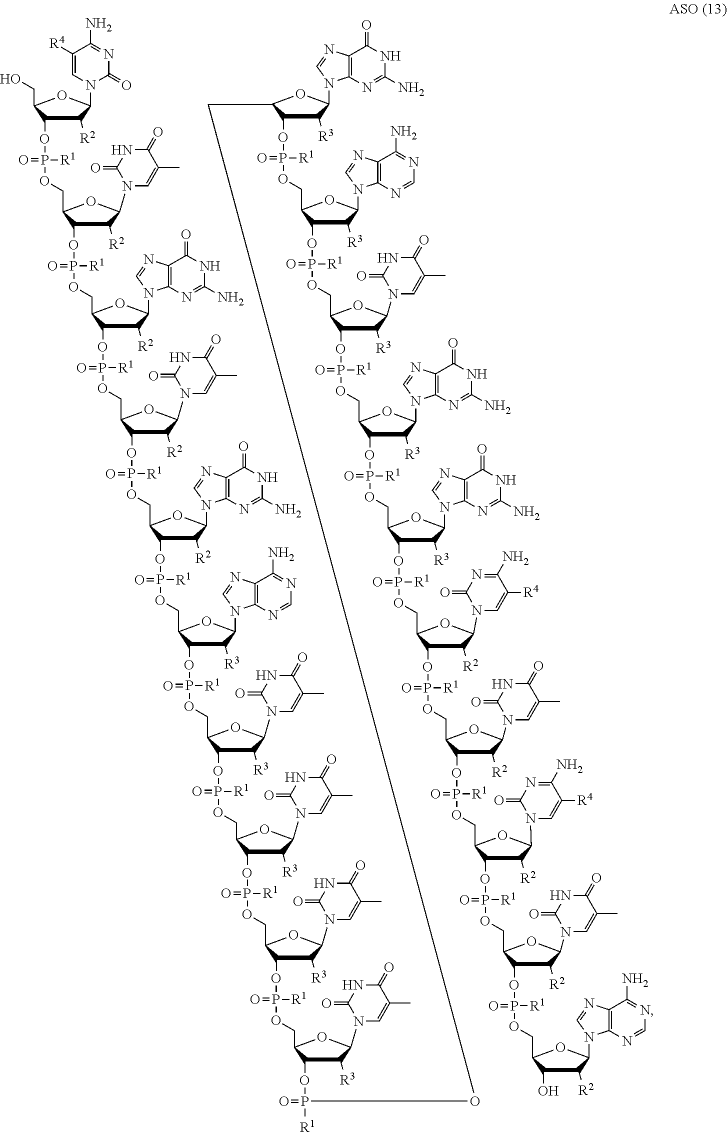

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH. OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (14)) according to the formula:

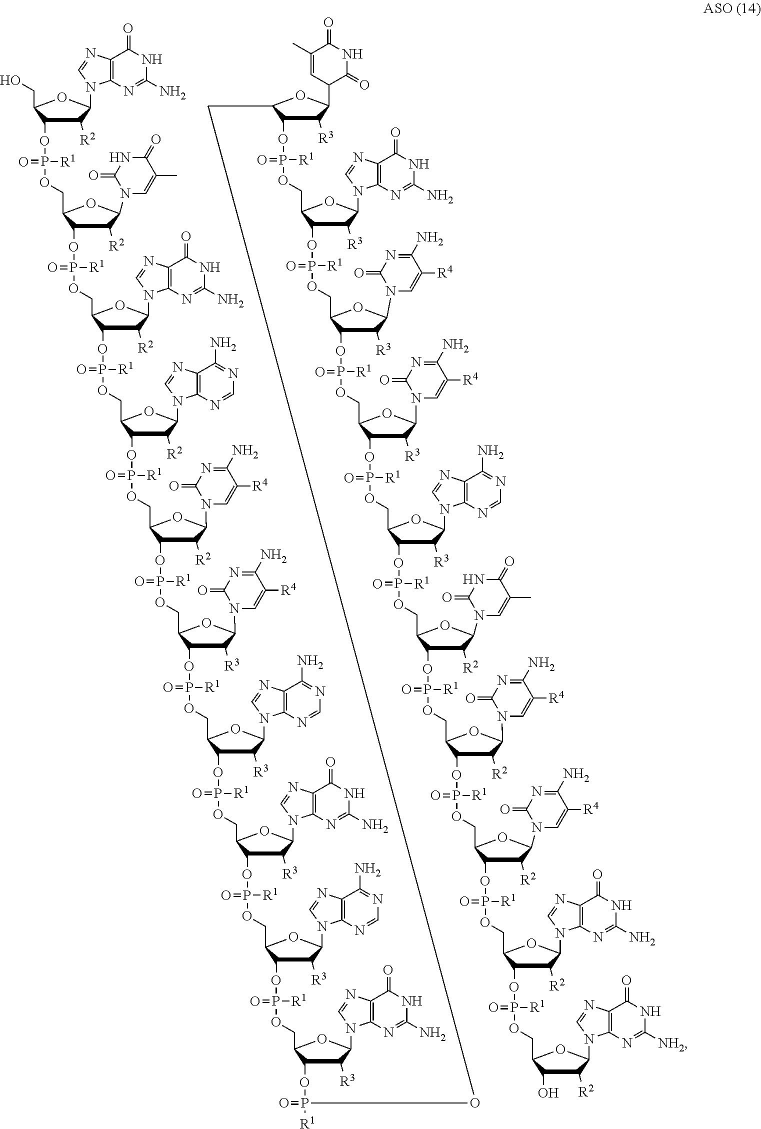

or a salt thereof, wherein:
each R¹ is independently selected from the group consisting of OH and SH;
each R² is independently selected from the group consisting of hydrogen, halo, OCH₃, OCF₃, OCH₂CH₃, OCH₁H₂CH₂OCH₃, and OCH-2CF₃; each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (15)) according to the formula:

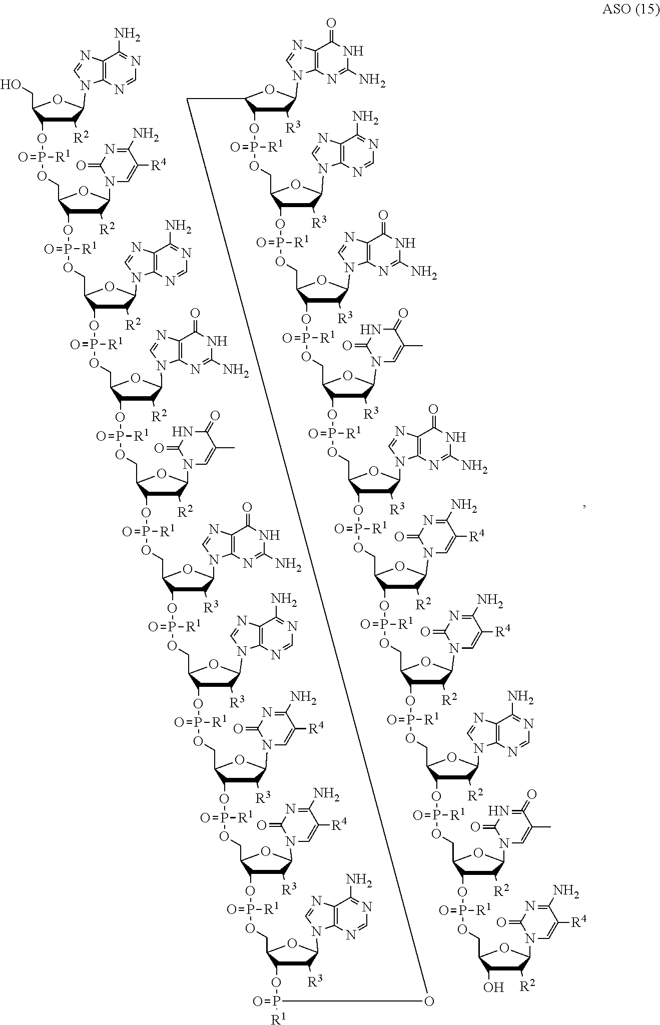

or a salt thereof, wherein:
each R¹ is independently selected from the group consisting of OH and SH;
each R² is independently selected from the group consisting of hydrogen, halo. OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (16)) according to the formula:

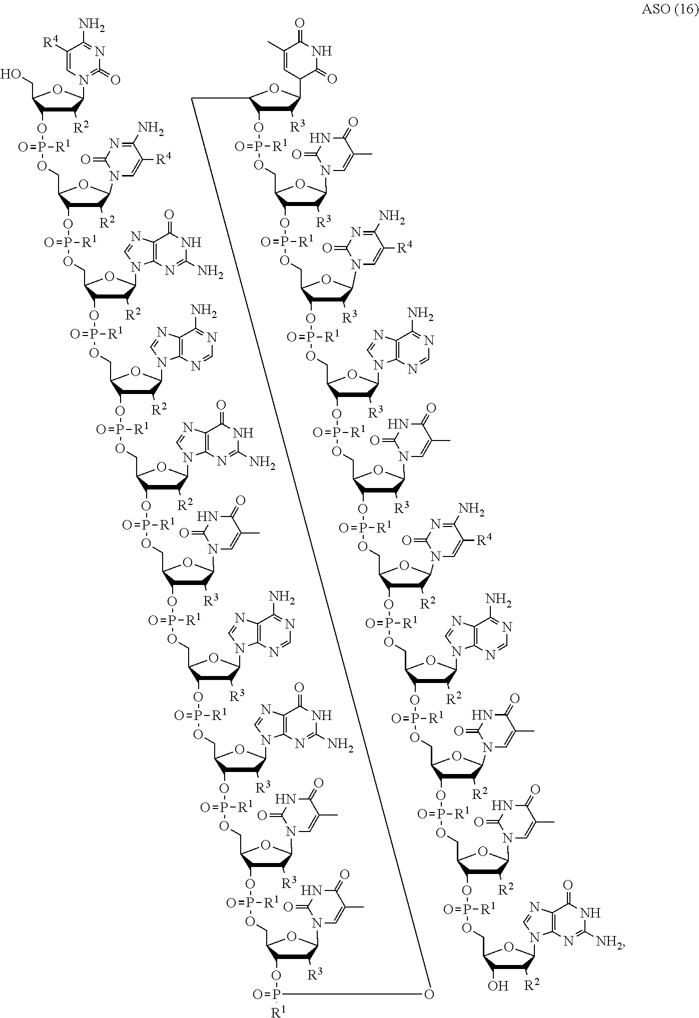

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃ and OCH₂CF₃; each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, is CH₃.

One embodiment provides an oligomeric compound (formula ASO (17)) according to the formula:

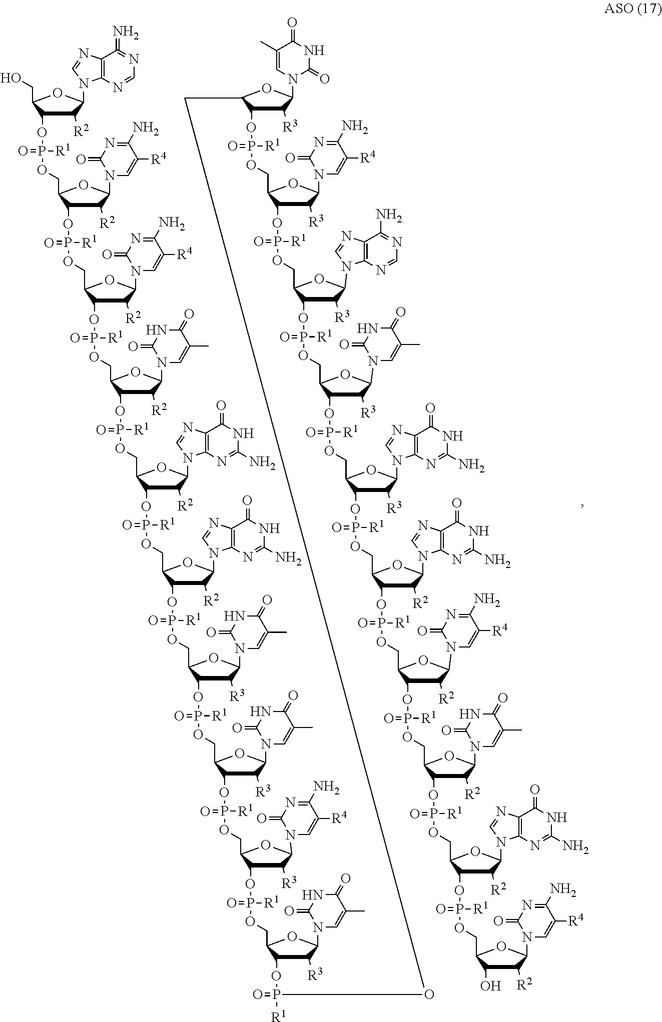

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (18)) according to the formula:

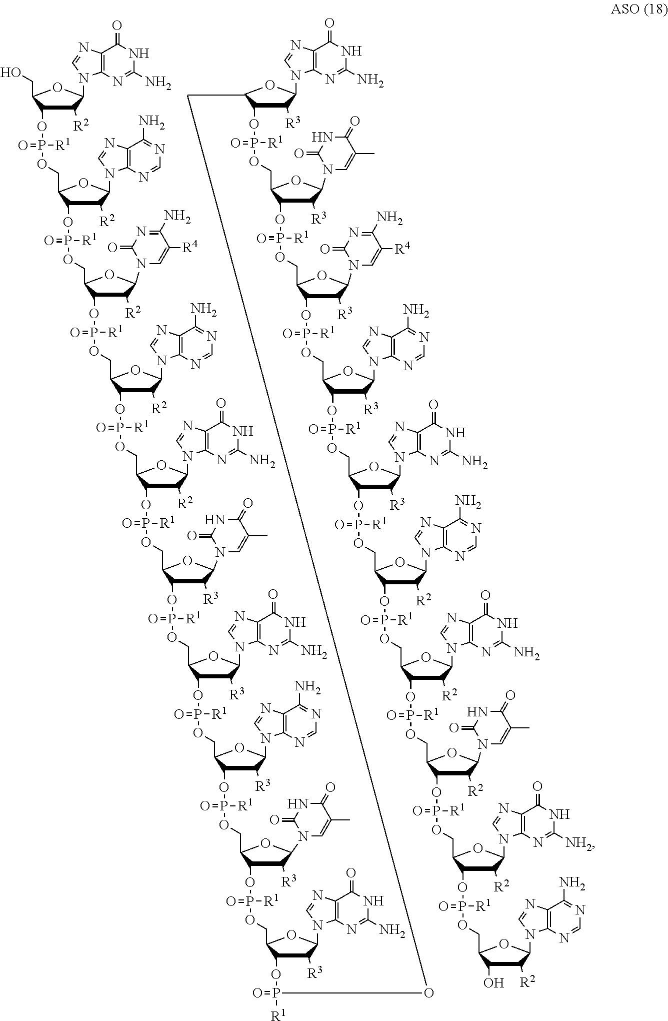

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides an oligomeric compound (formula ASO (19)) according to the formula:

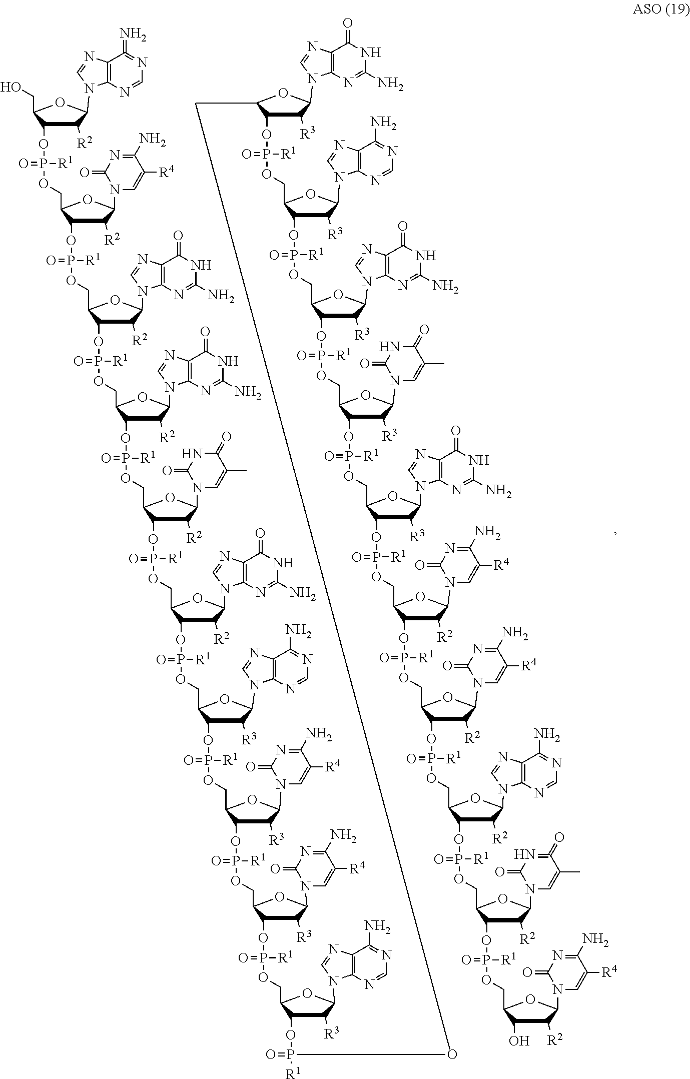

or a salt thereof, wherein:
each R¹ is independently selected from the group consisting of 01H and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃. In some embodiments, R⁴ is hydrogen. In some embodiments, R⁴ is CH₃.

One embodiment provides a method of treating a disease associated with runt-related transcription factor 1 (RUNX1) in a subject in need thereof, comprising administering an oligomeric compound, or a salt thereof, to the subject, said oligomeric compound comprising a modified oligonucleotide consisting of 17-23 linked nucleosides, wherein the modified oligonucleotide has a nucleobase sequence that is at least 80% complementary to any contiguous 17-23 nucleobase sequences of RUNX1 mRNA or RUNX1 gene, when measured across the entire nucleobase sequence of the modified oligonucleotide, wherein the modified oligonucleotide comprises at least one modification selected from a modified sugar, a sugar surrogate, and a modified internucleoside linkage, and wherein the RUNX1 gene comprises a sense strand and an antisense strand.

One embodiment provides a method of treating a disease associated with runt-related transcription factor 1 (RUNX1) in a subject in need thereof, comprising administering an inhibitor of runt-related transcription factor 1 (RUNX1).

One embodiment provides a method of treating a disease associated with runt-related transcription factor 1 (RUNX1) in a subject in need thereof, comprising administering an inhibitor of runt-related transcription factor 1 (RUNX1) synthesis.

One embodiment provides a method of improving cognitive function in a subject having an overexpression of RUNX1 in a cell, comprising administering an inhibitor of runt-related transcription factor 1 (RUNX1) synthesis to the subject.

One embodiment provides a method of improving cognitive function in a Down syndrome subject comprising identifying a Down syndrome subject in need of treatment for cognitive disability and administering to the subject a therapeutically effective amount of a RUNX1 inhibitor thereby reducing the expression of RUNX1 in the subject's microglia and increasing cognitive function in the subject.

One embodiment provides a method of treating cognitive loss associated with a neurodegenerative disease or disorder, the method comprising: identifying a subject in need of treatment for cognitive decline; and administering a therapeutically effective amount of a RUNX1 inhibitor to the subject thereby reducing the expression of RUNX1 in microglia of the subject and treating the cognitive loss in the subject.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 shows a modified antisense oligonucleotide (Compound 1) of SEQ ID NO: 20 for use in modulating expression levels of a human RUNX1 nucleic acid sequence comprising SEQ ID NO: 1.

FIG. 2 shows a modified antisense oligonucleotide (Compound 2) of SEQ ID NO: 21 for use in modulating expression levels of RUNX1 comprising SEQ ID NO: 2.

FIG. 3 shows a modified antisense oligonucleotide (Compound 3) of SEQ ID NO: 22 for use in modulating expression levels of a human RUNX1 nucleic acid sequence comprising SEQ ID NO: 3.

FIG. 4 shows a modified antisense oligonucleotide (Compound 4) of SEQ ID NO: 23 for use in modulating expression levels of a human RUNX1 nucleic acid sequence comprising SEQ ID NO: 4.

FIG. 5 shows a modified antisense oligonucleotide (Compound 5) of SEQ ID NO: 24 for use in modulating expression levels of a human RUNX1 nucleic acid sequence comprising SEQ ID NO: 5.

FIG. 6 shows a modified antisense oligonucleotide (Compound 6) of SEQ ID NO: 25 for use in modulating expression levels of a human RUNX1 nucleic acid sequence comprising SEQ ID NO: 6.

FIG. 7 shows a modified antisense oligonucleotide (Compound 7) of SEQ ID NO: 26 for use in modulating expression levels of a human RUNX1 nucleic acid sequence comprising SEQ ID NO: 7.

FIG. 8 shows a modified antisense oligonucleotide (Compound 8) of SEQ ID NO: 27 for use in modulating expression levels of a human RUNX1 nucleic acid sequence comprising SEQ ID NO: 8.

FIG. 9 shows a modified antisense oligonucleotide (Compound 9) of SEQ ID NO: 28 for use in modulating expression levels of a human RUNX1 nucleic acid sequence comprising SEQ ID NO: 9.

FIG. 10 shows a modified antisense oligonucleotide (Compound 10) of SEQ ID NO: 29 for use in modulating expression levels of a human RUNX1 nucleic acid sequence comprising SEQ ID NO: 10.

FIG. 11 shows a modified antisense oligonucleotide (Compound 11) of SEQ ID NO: 30 for use in modulating expression levels of a human RUNX1 nucleic acid sequence comprising SEQ ID NO: 11.

FIG. 12 shows a modified antisense oligonucleotide (Compound 12) of SEQ ID NO: 31 for use in modulating expression levels of a human RUTNX1 nucleic acid sequence comprising SEQ ID NO: 12.

FIG. 13 shows a modified antisense oligonucleotide (Compound 13) of SEQ ID NO: 32 for use in modulating expression levels of a human RUNX1 nucleic acid sequence comprising SEQ ID NO: 13.

FIG. 14 shows a modified antisense oligonucleotide (Compound 14) of SEQ ID NO: 33 for use in modulating expression levels of a human RUTNX1 nucleic acid sequence comprising SEQ ID NO: 14.

FIG. 15 shows a modified antisense oligonucleotide (Compound 15) of SEQ ID NO: 34 for use in modulating expression levels of a human RUNX1 nucleic acid sequence comprising SEQ ID NO: 15.

FIG. 16 shows a modified antisense oligonucleotide (Compound 16) of SEQ ID NO: 35 for use in modulating expression levels of a human RUNX1 nucleic acid sequence comprising SEQ ID NO: 16.

FIG. 17 shows a modified antisense oligonucleotide (Compound 17) of SEQ ID NO: 36 for use in modulating expression levels of a human RUNX1 nucleic acid sequence comprising SEQ ID NO: 17.

FIG. 18 shows a modified antisense oligonucleotide (Compound 18) of SEQ ID NO: 37 for use in modulating expression levels of a human RUNX1 nucleic acid sequence comprising SEQ TD NO: 18.

FIG. 19 shows a modified antisense oligonucleotide (Compound 19) of SEQ ID NO: 38 for use in modulating expression levels of a human RUTNX1 nucleic acid sequence comprising SEQ ID NO: 19.

FIG. 20A and FIG. 20B show the effect of an exemplary modified antisense oligonucleotide on mouse transcript knockdown and mouse protein knockdown in vitro.

FIG. 21 shows the effect of an exemplary modified antisense oligonucleotide on human transcript knockdown in vitro.

FIG. 22 shows the effect of an exemplary modified antisense oligonucleotide on mouse transcript knockdown in vivo.

FIG. 23 shows the effect of an exemplary modified antisense oligonucleotide on TNF-alpha, CCL3, CCL5, and CCL20.

FIG. 24 shows the effect of an exemplary modified antisense oligonucleotide on C1qa, C1qb, and C1qc.

### DETAILED DESCRIPTION OF THE INVENTION

As used herein and in the appended claims, the singular forms “a.” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“2′-O-methoxyethyl” (also 2′-MOE and 2′-OCH₂CH₂OCH₃ and MOE) refers to an 0-methoxy-ethyl modification of the 2′ position of a furanose ring. A 2′-O-methoxyethyl modified sugar is a modified sugar.

“2′-MOE nucleoside” (also 2′-O-methoxyethyl nucleoside) means a nucleoside comprising a MOE modified sugar moiety.

“2′-substituted nucleoside” means a nucleoside comprising a substituent at the 2′-position of the furanose ring other than H or OH. In certain embodiments, 2′-substituted nucleosides include nucleosides with bicyclic sugar modifications.

“5-methylcytosine” means a cytosine modified with a methyl group attached to the 5′ position. A 5-methylcytosine is a modified nucleobase.

“About” means within +7% of a value. For example, if it is stated, “the compounds affected at least about 70% inhibition of RUNX1 antisense transcript”, it is implied that the RUNX1 antisense transcript levels are inhibited within a range of 63% and 77%.

“Administered concomitantly” refers to the co-administration of two pharmaceutical agents in any manner in which the pharmacological effects of both are manifest in the patient at the same time. Concomitant administration does not require that both pharmaceutical agents be administered in a single pharmaceutical composition, in the same dosage form, or by the same route of administration. The effects of both pharmaceutical agents need not manifest themselves at the same time. The effects need only be overlapping for a period of time and need not be coextensive.

“Administering” means providing a pharmaceutical agent to an animal, and includes, but is not limited to administering by a medical professional and self-administering.

“Amelioration” refers to a lessening, slowing, stopping, or reversing of at least one indicator of the severity of a condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fe region.

“Antisense activity” means any detectable or measurable activity attributable to the hybridization of an antisense compound to its target nucleic acid. In certain embodiments, antisense activity is a decrease in the amount or expression of a target nucleic acid or protein product encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels or in the absence of the antisense compound.

“Antisense mechanisms” are all those mechanisms involving hybridization of a compound with a target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“RUNX1 antisense transcript” means transcripts produced from the non-coding strand (also antisense strand and template strand) of the RUNX1 gene. The RUNX1 antisense transcript differs from the canonically transcribed “RUNX1 sense transcript”, which is produced from the coding strand (also sense strand) of the RUNX1 gene.

“RUNX1 antisense transcript associated RAN translation products” means aberrant peptide or di-peptide polymers translated through RAN translation (i.e., repeat-associated, and non-ATG-dependent translation). In certain embodiments, the RUNX1 antisense transcript associated RAN translation products are any of poly-(proline-alanine), poly-(proline-arginine), and poly-(proline-glycine).

“RUNX1 antisense transcript specific inhibitor” refers to any agent capable of specifically inhibiting the expression of RUNX1 antisense transcript and/or its expression products at the molecular level. For example, RUNX1 specific antisense transcript inhibitors include nucleic acids (including antisense compounds), siRNAs, aptamers, antibodies, peptides, small molecules, and other agents capable of inhibiting the expression of RUNX1 antisense transcript and/or its expression products, such as RUNX1 antisense transcript associated RAN translation products.

“RUNX1 associated condition” means any condition associated with any RUNX1 nucleic acid or expression product thereof, regardless of which DNA strand the RUNX1 nucleic acid or expression product thereof is derived from. Such conditions may include genetic disorders. Such genetic disorders may include Down syndrome (DS).

“RUNX1 nucleic acid” means any nucleic acid derived from the RUNX1 locus, regardless of which DNA strand the RUNX1 nucleic acid is derived from. In certain embodiments, a RUNX1 nucleic acid includes a DNA sequence encoding RUNX1, an RNA sequence transcribed from DNA encoding RUNX1 including genomic DNA comprising introns and exons (i.e., pre-mRNA), and an mRNA sequence encoding RUNX1. “RUNX1 mRNA” means an mRNA encoding a RUNX1 protein. In certain embodiments, a RUNX1 nucleic acid includes transcripts produced from the coding strand of the RUNX1 gene. RUNX1 sense transcripts are examples of RUNX1 nucleic acids. In certain embodiments, a RUNX1 nucleic acid includes transcripts produced from the non-coding strand of the RUXN1 gene. RUNX1 antisense transcripts are examples of RUNX1 nucleic acids.

“RUNX1 transcript” means an RNA transcribed from RUNX1. In certain embodiments, a RUNX1 transcript is a RUNX1 sense transcript. In certain embodiments, a RUNX1 transcript is a RUNX1 antisense transcript.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′—CH(CH₃)—O-2′ bridge.

“Chemically distinct region” refers to a region of an antisense compound that is in some way chemically different than another region of the same antisense compound. For example, a region having 2′-O-methoxyethyl nucleosides is chemically distinct from a region having nucleosides without 2′-O-methoxyethyl modifications.

“Chimeric antisense compound” means an antisense compound that has at least two chemically distinct regions, each position having a plurality of subunits.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid

“Comprise,” “comprises.” and “comprising” will be understood to imply the inclusion of a stated step or element or group of steps or elements but not the exclusion of any other step or element or group of steps or elements.

“Contiguous nucleobases” means nucleobases immediately adjacent to each other.

“Designing” or “designed to” refer to the process of designing an oligomeric compound that specifically hybridizes with a selected nucleic acid molecule.

“Diluent” means an ingredient in a composition that lacks pharmacological activity, but is pharmaceutically necessary or desirable. For example, in drugs that are injected, the diluent may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” in the context of modulating an activity or of treating or preventing a condition means the administration of that amount of pharmaceutical agent to a subject in need of such modulation, treatment, or prophylaxis, either in a single dose or as part of a series, that is effective for modulation of that effect, or for treatment or prophylaxis or improvement of that condition. The effective amount may vary among individuals depending on the health and physical condition of the individual to be treated, the taxonomic group of the individuals to be treated, the formulation of the composition, assessment of the individuals medical condition, and other relevant factors.

“Efficacy” means the ability to produce a desired effect.

“Expression” includes all the functions by which a gene's coded information, regardless of which DNA strand the coded information is derived from, is converted into structures present and operating in a cell. Such structures include, but are not limited to the products of transcription and translation, including RAN translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” and the external regions may be referred to as the “wings.”

“Gap-narrowed” means a chimeric antisense compound having a gap segment of 9 or fewer contiguous 2′-deoxyribonucleosides positioned between and immediately adjacent to 5′ and 3′ wing segments having from 1 to 6 nucleosides.

“Gap-widened” means a chimeric antisense compound having a gap segment of 12 or more contiguous 2′-deoxyribonucleosides positioned between and immediately adjacent to 5′ and 3′ wing segments having from 1 to 6 nucleosides.

“Hybridization” means the annealing of complementary nucleic acid molecules. In certain embodiments, complementary nucleic acid molecules include, but are not limited to, an antisense compound and a target nucleic acid. In certain embodiments, complementary nucleic acid molecules include, but are not limited to, an antisense oligonucleotide and a nucleic acid target.

“Immediately adjacent” means there are no intervening elements between the immediately adjacent elements.

“Individual” means a human or non-human animal selected for treatment or therapy.

“Inhibiting expression of a RUNX1 antisense transcript” means reducing the level or expression of a RUNX1 antisense transcript and/or its expression products (e.g., RAN translation products). In certain embodiments, RUTNX1 antisense transcripts are inhibited in the presence of an antisense compound targeting a RUNX1 antisense transcript, including an antisense oligonucleotide targeting a RUNX1 antisense transcript, as compared to expression of RUNX1 antisense transcript levels in the absence of a RUNX1 antisense compound, such as an antisense oligonucleotide.

“Inhibiting expression of a RUNX1 sense transcript” means reducing the level or expression of a RUNX1 sense transcript and/or its expression products (e.g., a RUNX1 mRNA and/or protein). In certain embodiments, RUNX1 sense transcripts are inhibited in the presence of an antisense compound targeting a RUNX1 sense transcript, including an antisense oligonucleotide targeting a RUNX1 sense transcript, as compared to expression of RUNX1 sense transcript levels in the absence of a RUNX1 antisense compound, such as an antisense oligonucleotide.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, and/or modified nucleobase.

“Modified oligonucleotide” means an oligonucleotide comprising at least one modified internucleoside linkage, modified sugar, and/or modified nucleobase.

“Modified sugar” means substitution and/or any change from a natural sugar moiety.

“Monomer” means a single unit of an oligomer. Monomers include, but are not limited to, nucleosides and nucleotides, whether naturally occurring or modified.

“Motif” means the pattern of unmodified and modified nucleoside in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarily” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Nucleotide” means a nucleoside having a phosphate group covalently linked to the sugar portion of the nucleoside.

“Off-target effect” refers to an unwanted or deleterious biological effect associated with modulation of RNA or protein expression of a gene other than the intended target nucleic acid.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, peptide refers to polypeptides and proteins.

“Pharmaceutical agent” means a substance that provides a therapeutic benefit when administered to an individual. In certain embodiments, an antisense oligonucleotide targeted to RUNX1 sense transcript is a pharmaceutical agent. In certain embodiments, an antisense oligonucleotide targeted to RUNX1 antisense transcript is a pharmaceutical agent.

“Pharmaceutical composition” means a mixture of substances suitable for administering to as subject. For example, a pharmaceutical composition may comprise an antisense oligonucleotide and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disease, disorder, or condition for a period of time from minutes to days, weeks to months, or indefinitely.

“Prodrug” means a therapeutic agent that is prepared in an inactive form that is converted to an active form within the body or cells thereof by the action of endogenous enzymes or other chemicals or conditions.

“Prophylactically effective amount” refers to an amount of a pharmaceutical agent that provides a prophylactic or preventative benefit to an animal.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Salts” mean a physiologically and pharmaceutically acceptable salts of antisense compounds, i.e., salts that retain the desired biological activity of the parent oligonucleotide and do not impart undesired toxicological effects thereto.

“Segments” are defined as smaller or sub-portions of regions within a target nucleic acid.

“Shortened” or “truncated” versions of antisense oligonucleotides taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies.

“Single-stranded oligonucleotide” means an oligonucleotide which is not hybridized to a complementary strand.

“Sites,” as used herein, are defined as unique nucleobase positions within a target nucleic acid.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Stringent hybridization conditions” or “stringent conditions” refer to conditions under which an oligomeric compound will hybridize to its target sequence, but to a minimal number of other sequences.

“Subject” means a human or non-human animal selected for treatment or therapy.

“Targeting” or “targeted” means the process of design and selection of an antisense compound that will specifically hybridize to a target nucleic acid and induce a desired effect.

“Target nucleic acid,” “target RNA,” and “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” or “treating” or “treatment” means administering a composition to effect an alteration or improvement of a disease, condition, or disorder.

“Unmodified nucleobases” means the purine bases adenine (A) and guanine (G), and the pyrimidine bases (T), cytosine (C), and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. (3-D-ribonucleosides) or a DNA nucleotide (i.e. O-D-deoxyribonucleoside).

“Wing segment” means a plurality of nucleosides modified to impart to an oligonucleotide properties such as enhanced inhibitory activity, increased binding affinity for a target nucleic acid, or resistance to degradation by in vivo nucleases.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, S, ³⁵S, ³⁶S, ³⁵Cl, ³⁷Cl, ⁷⁹Br, ⁸¹Br, ¹²⁵I are all contemplated. In some embodiments, isotopic substitution with 18F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

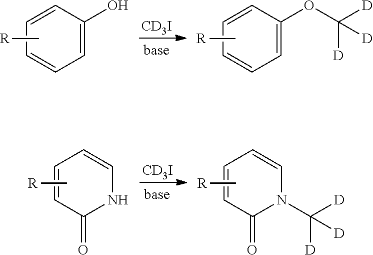

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain seven deuterium atoms. In another embodiment, the compounds disclosed herein contain more than seven deuterium atoms. In another embodiment, the compounds disclosed herein contain more than eight deuterium atoms. In another embodiment, the compounds disclosed herein contain more than nine deuterium atoms. In another embodiment, the compounds disclosed herein contain more than ten deuterium atoms. In another embodiment, the compounds disclosed herein contain more than eleven deuterium atoms. In another embodiment, the compounds disclosed herein contain more than twelve deuterium atoms. In another embodiment, the compounds disclosed herein contain more than thirteen deuterium atoms. In another embodiment, the compounds disclosed herein contain more than fourteen deuterium atoms. In another embodiment, the compounds disclosed herein contain more than fifteen deuterium atoms. In another embodiment, the compounds disclosed herein contain more than sixteen deuterium atoms. In another embodiment, the compounds disclosed herein contain more than seventeen deuterium atoms. In another embodiment, the compounds disclosed herein contain more than eighteen deuterium atoms. In another embodiment, the compounds disclosed herein contain more than nineteen deuterium atoms. In another embodiment, the compounds disclosed herein contain more than twenty deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starling material. In one embodiment, the compounds described herein contain one deuterium atom per oligonucleoside residue. In one embodiment, the compounds described herein contain two deuterium atoms per oligonucleoside residue. In one embodiment, the compounds described herein contain more than two deuterium atoms per oligonucleoside residue.

### RUNT-Related Transcription Factor 1

Runt-related Transcription Factor 1 (RUNX1) is a transcription factor expressed in immune cells throughout the body. It regulates differentiation of hematopoietic stem cells into mature blood cells, and is important in the development of neurons transmitting pain. Modulation of the expression of RUNX1 levels allows for modification of transcriptomic, morphological, and functional aspects of microglia. Normalization of microglia in brains of individuals with Down syndrome has therapeutic indications.

### Related Art RUNX1

WO/2012/1 13555 discloses MYI-10 as a new marker for RUNX1 inactivation. WO/2014/199377 discloses a method of treating a hematological malignancy associated with RUNX1 activity by administering an agent that downregulates an activity or expression of RUNX1. WO/2016/025744 discloses compounds that bind and inhibit binding of wild-type CBFβ to RUNX1. WO/2021/216378 discloses methods of treating viral infections by inhibiting RUNX1 or CBFβ. WO/2005/089080 discloses RUNX1 as a marker for screening autoimmune disorders. WO/2012/153854 discloses means for modulating the expression of genes including RUNX1 associated with inflammation and infection. WO/2007/082777 discloses a novel C-terminal exon of RUNX1/AML1 for the treatment of various diseases. WO/2019/024579 discloses the content of 5-hydroxymethylcytosine in RUNX1 as a marker for lung cancer. WO/2012/125787 discloses a compound that inhibits CBFβ and RUNX1 binding in a subject with CBF leukemia. WO/2019/127664 discloses multipotent stem cells comprising a RUNX1 and HOXA9 tandem co-expression vector. WO/2019/099595 discloses a nano-emulsion composition comprising a CBFβ-RUNX1 inhibitor. WO/2022/086476 discloses RUNX1 as a marker for diagnosing Philadelphia-like acute lymphoblastic leukemia. WO/2006/009629 discloses methods for treating muscular dystrophy in an individual by administering an agent that increases expression of RUNX1 in the individual. WO/2020/017676 discloses a therapeutic stem cell composition that can comprise RUNX1. WO/2006/096498 discloses regulation of RUNX1 for treatment of pain. WO/2020/022899 discloses vaccines that bring out-of-frame sequences of RUNX1 in-frame. WO/2014/177464 discloses inhibitors of RUNX1 as tumor therapeutics. WO/2019/040448 discloses methods of inducing HSC specification in a cell by contacting with a RUNX1-CBFβ inhibitor. WO/2012/166722 discloses RUNX1 as a marker that can be used to determine appropriate chemotherapy for a subject. WO/2019/099560 discloses inhibition of RUNX1 for treatment of proliferative vitreoretinopathy and conditions associated with epithelial to mesenchymal transition. WO/2019/084662 discloses heterocyclic compounds for treatment of acute myeloid leukemia characterized by certain features including mutated RUNX1. WO/2018/023197 discloses compounds for treatment of acute myeloid leukemia characterized by certain features including mutated RUNX1. WO/2016/182904 discloses RUNX1 pathway impairment as a marker for selection and treatment of patients with a tumor or cancer with cortistatin.

Currently, there is no technology capable of improving memory or selectively modifying microglial activation states in the brain or CNS of individuals afflicted with Down syndrome (DS). There is a need for treatments that could enable improvement of cognitive aspects of DS that currently have no available treatment. Despite the existence of numerous ASOs designed to target other genes in an effort to treat neurological diseases, none are designed to target RUNX1 or modify microglial behavior in Down syndrome or in people with dementia.

### Antisense Compounds

One embodiment provides an oligomeric compound, or a salt thereof, comprising a modified oligonucleotide consisting of 10-30 linked nucleosides wherein the modified oligonucleotide has a nucleobase sequence that is at least 80% complementary to any of the nucleobase sequences of SEQ ID NO: 1-19, when measured across the entire nucleobase sequence of the modified oligonucleotide, and wherein the modified oligonucleotide comprises at least one modification selected from a modified sugar, a sugar surrogate, and a modified internucleoside linkage. In some embodiments, the modified oligonucleotide comprises at least two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage. In some embodiments, the modified oligonucleotide comprises at least ten, eleven, twelve, thirteen, fourteen, fifteen, sixteen, seventeen, eighteen, nineteen, twenty, 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage. In some embodiments, the modified oligonucleotide comprises at least 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage. In some embodiments, the modified oligonucleotide comprises at least 28, 29, 30, 31 or 32 modifications, or number of modifications within a range defined by any of the preceding numbers, selected from a modified sugar, a sugar surrogate, and a modified internucleoside linkage. In some embodiments, the modified oligonucleotide comprises a modified region of at least 10 contiguous modified nucleosides. In some embodiments, the modified oligonucleotide comprises at least one 2′-substituted modified sugar moiety. In some embodiments, the modified oligonucleotide comprises at least one bicyclic modified sugar moiety. In some embodiments, the modified oligonucleotide comprises a plurality of internucleoside linkages and each internucleoside linkage is either a phosphodiester internucleoside linkage or a phosphorothioate internucleoside linkage. In some embodiments, the at least one modified internucleoside linkage is a phosphorothioate internucleoside linkage. In some embodiments, the antisense compound comprises at least one conjugate group. In some embodiments, the antisense compound is single-stranded. In some embodiments, the modified oligonucleotide is at least 90% complementary to any of the nucleobase sequences of SEQ ID NO: 1-19. In some embodiments, the modified oligonucleotide comprises a plurality of 2′-O-methyl modified sugar moieties. In some embodiments, the modified oligonucleotide is 100% complementary to any of the nucleobase sequences of SEQ ID NO: 1-19.

One embodiment provides an oligomeric compound, or a salt thereof, comprising a modified oligonucleotide consisting of 10-30 linked nucleosides wherein the modified oligonucleotide has a nucleobase sequence that is targeted to any of the nucleobase sequences of SEQ ID NO: 39-57, when measured across the entire nucleobase sequence of the modified oligonucleotide, and wherein the modified oligonucleotide comprises at least one modification selected from a modified sugar, a sugar surrogate, and a modified internucleoside linkage. In some embodiments, the modified oligonucleotide comprises at least two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage. In certain embodiments, the modified oligonucleotide comprises at least ten, eleven, twelve, thirteen, fourteen, fifteen, sixteen, seventeen, eighteen, nineteen, twenty, 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage. In some embodiments, the modified oligonucleotide comprises at least 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage. In certain embodiments, the modified oligonucleotide comprises at least 28, 29, 30, 31 or 32 modifications, or number of modifications within a range defined by any of the preceding numbers, selected from a modified sugar, a sugar surrogate, and a modified internucleoside linkage. In some embodiments, the modified oligonucleotide comprises a modified region of at least 10 contiguous modified nucleosides. In certain embodiments, the modified oligonucleotide comprises at least one 2-substituted modified sugar moiety. In some embodiments, the modified oligonucleotide comprises at least one bicyclic modified sugar moiety. In certain embodiments, the modified oligonucleotide comprises a plurality of internucleoside linkages and each internucleoside linkage is either a phosphodiester internucleoside linkage or a phosphorothioate internucleoside linkage. In some embodiments, the at least one modified internucleoside linkage is a phosphorothioate internucleoside linkage. In certain embodiments, the antisense compound comprises at least one conjugate group. In some embodiments, the antisense compound is single-stranded. In certain embodiments, the modified oligonucleotide is at least 90% complementary to any of the nucleobase sequences of SEQ ID NO: 39-57. In some embodiments, the modified oligonucleotide comprises a plurality of 2-O-methyl modified sugar moieties. In certain embodiments, the modified oligonucleotide is 100% complementary to any of the nucleobase sequences of SEQ ID NO: 39-57.

One embodiment provides an oligomeric compound formula ASO (I)) according to the following formula:

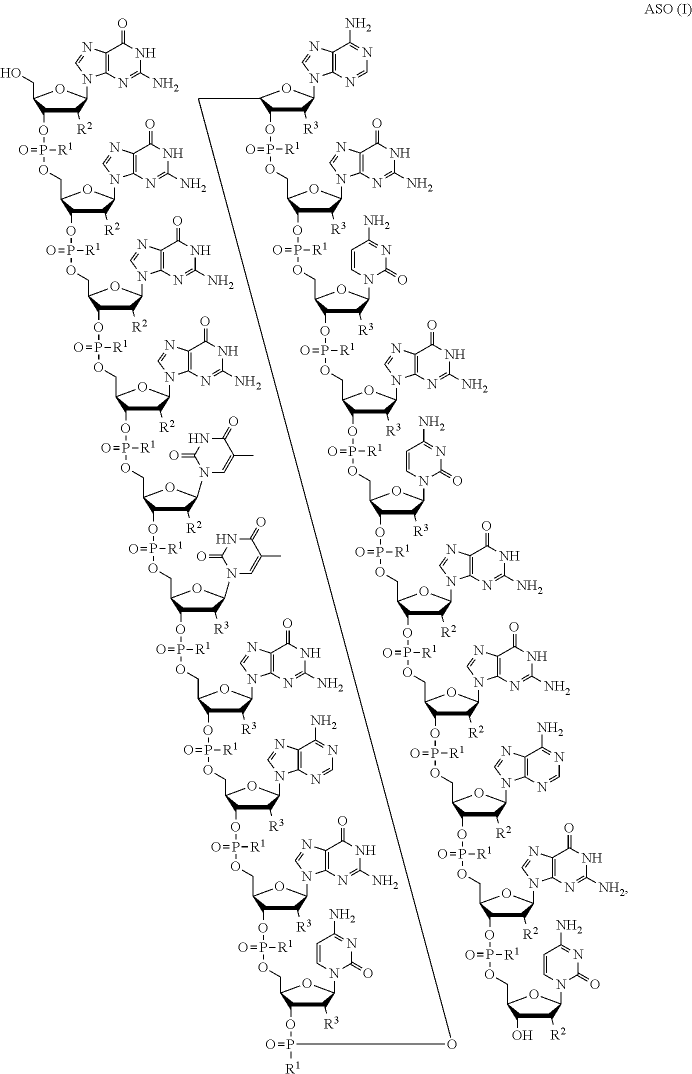

or a salt thereof, wherein:
each R¹ is independently selected from the group consisting of OH and SH; each R² is independently selected from the group consisting of hydrogen, halo, OCH₃,
OCF₃, OCH₂CH₃, OCH₂CH₂OCH₃, and OCH₂CF₃; and each R³ is independently selected from the group consisting of hydrogen, halo, OH,
OCH₃, OCF₃, OCH₂CH₃, OCH₂CH₂OCH₃, and OCH₂CF₃.

One embodiment provides an oligomeric compound (formula ASO (II)) according to the following formula:

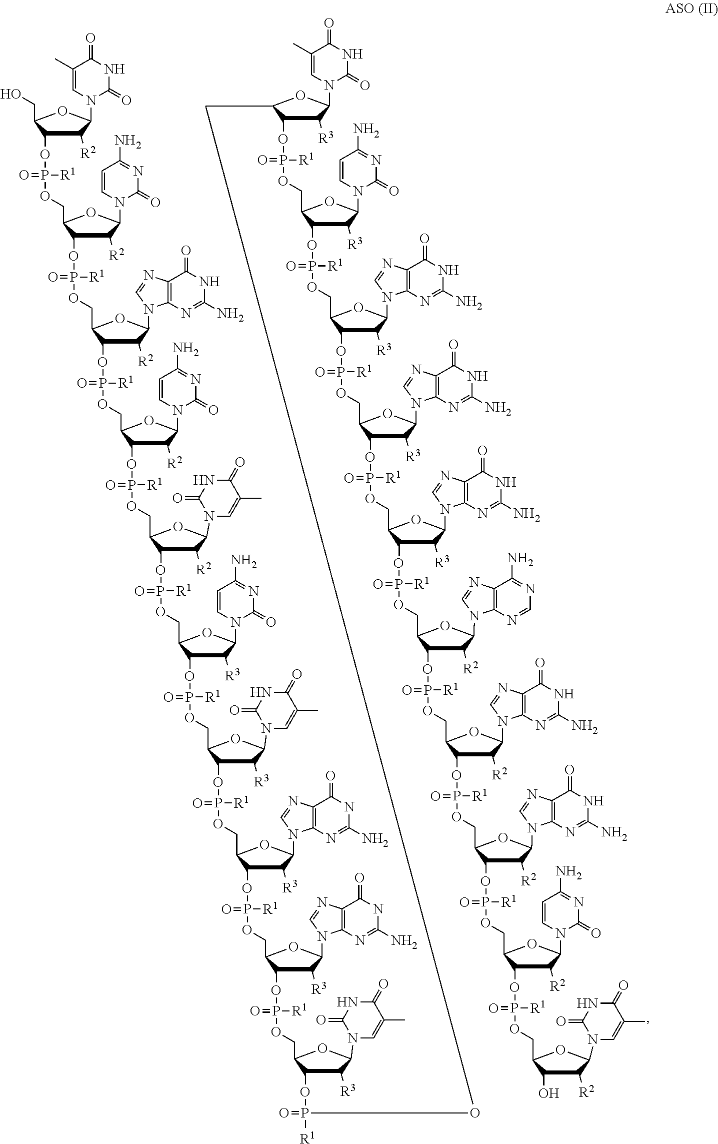

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (III)) according to the following formula:

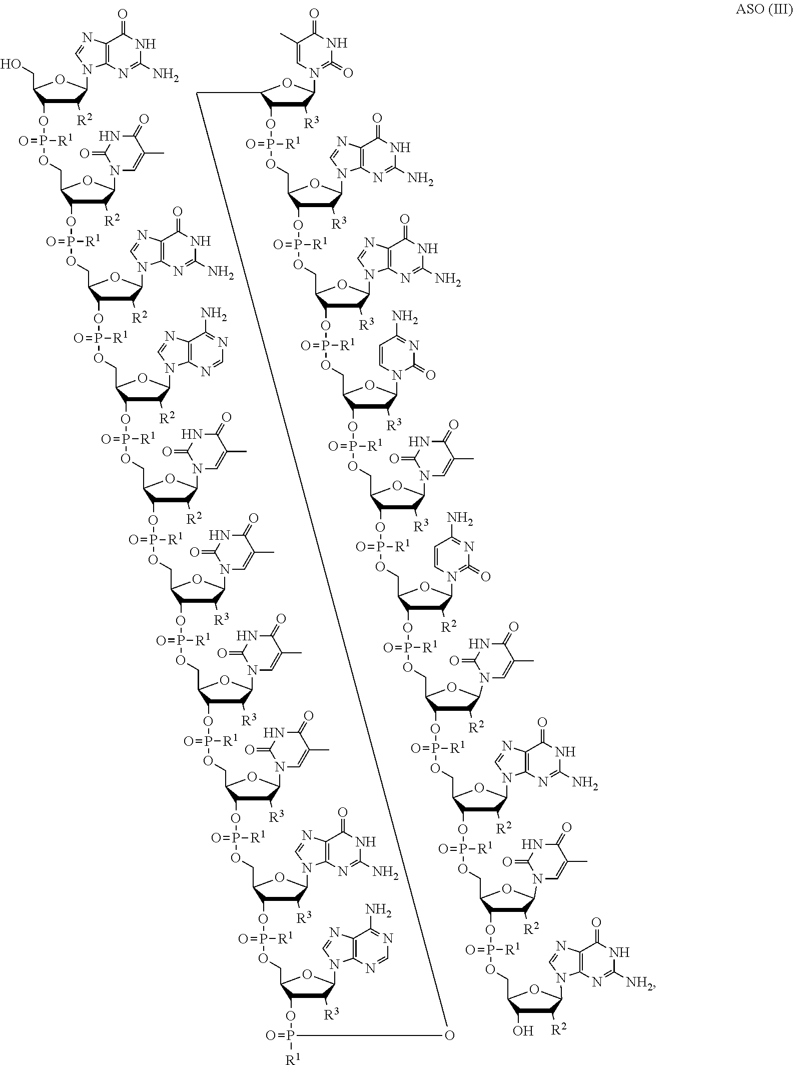

or a salt thereof wherein:
each R¹ is independently selected from the group consisting of OH and SH;
each R² is independently selected from the group consisting of hydrogen, halo, OCH₃, OCF₃, OH₂CH₃, OCH₂CH₂OCH₃, and OCH₂CF₃; and
each R³ is independently selected from the group consisting of hydrogen, halo, OHR OCH₃, OCF₃, OCH₂CH₃, OCH₂CH₂OCH₃, and OCH₂CF₃

One embodiment provides an oligomeric compound (formula ASO (IV)) according to the formula:

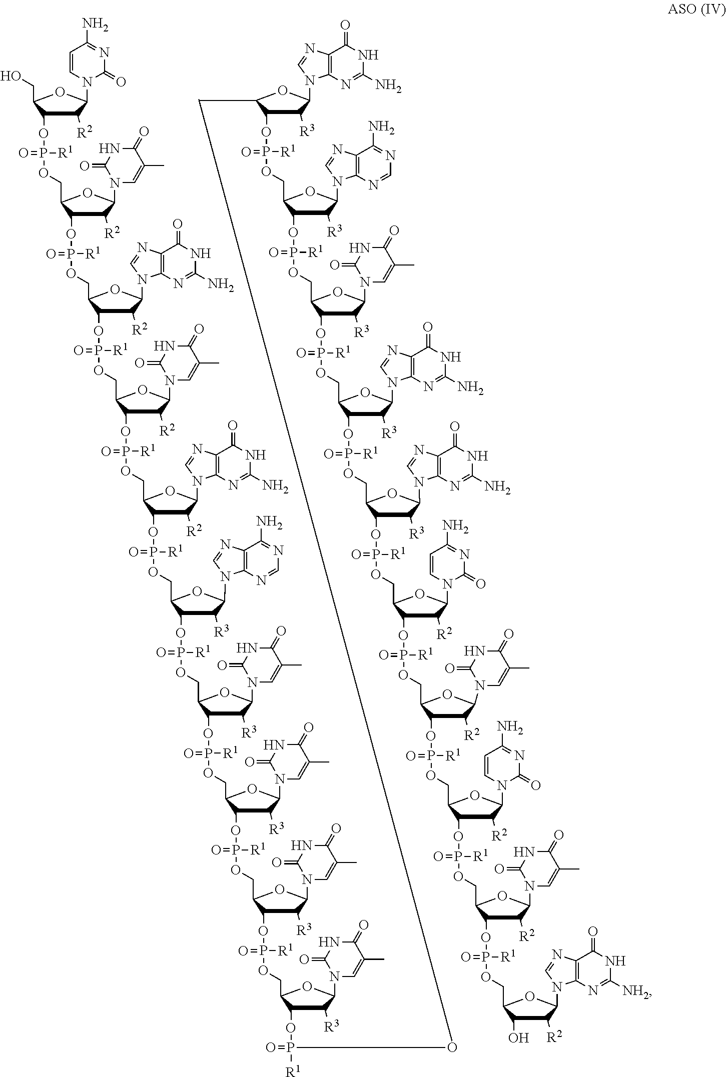

or a salt thereof wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (V)) according to the formula:

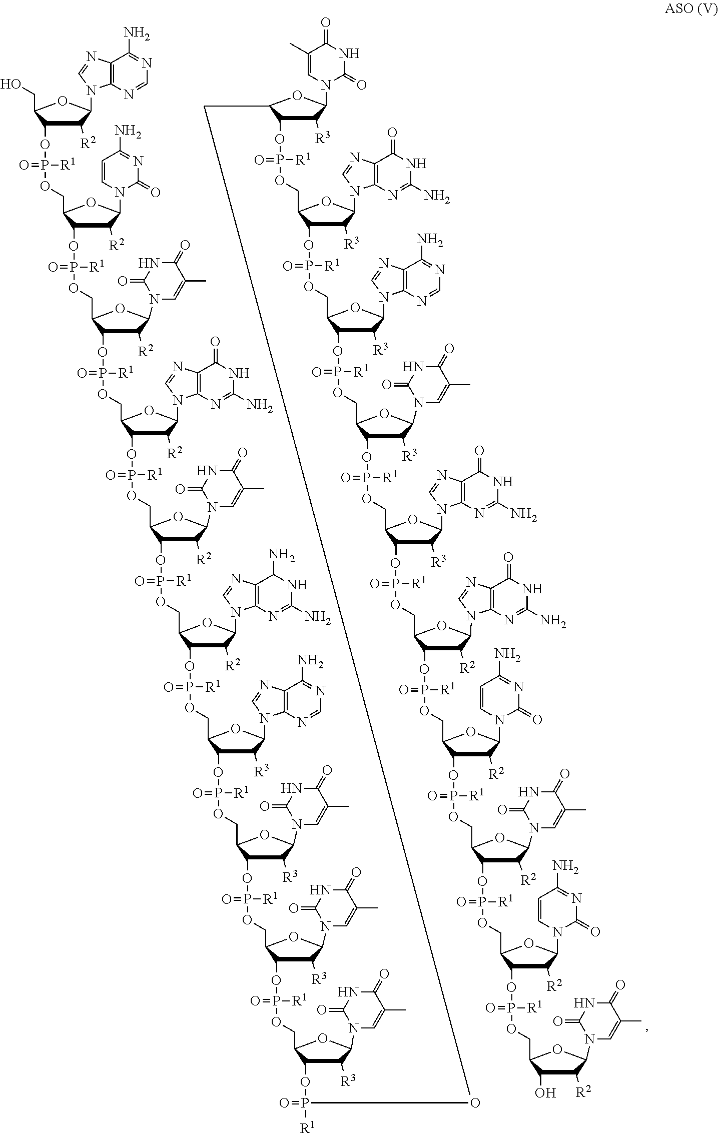

or a salt thereof, wherein:
each R¹ is independently selected from the group consisting of OH and SH;
each R² is independently selected from the group consisting of hydrogen, halo, OCH₃, OCF₃, OCH₁₂CH₃, OCH₄H₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (VI)) according to the formula:

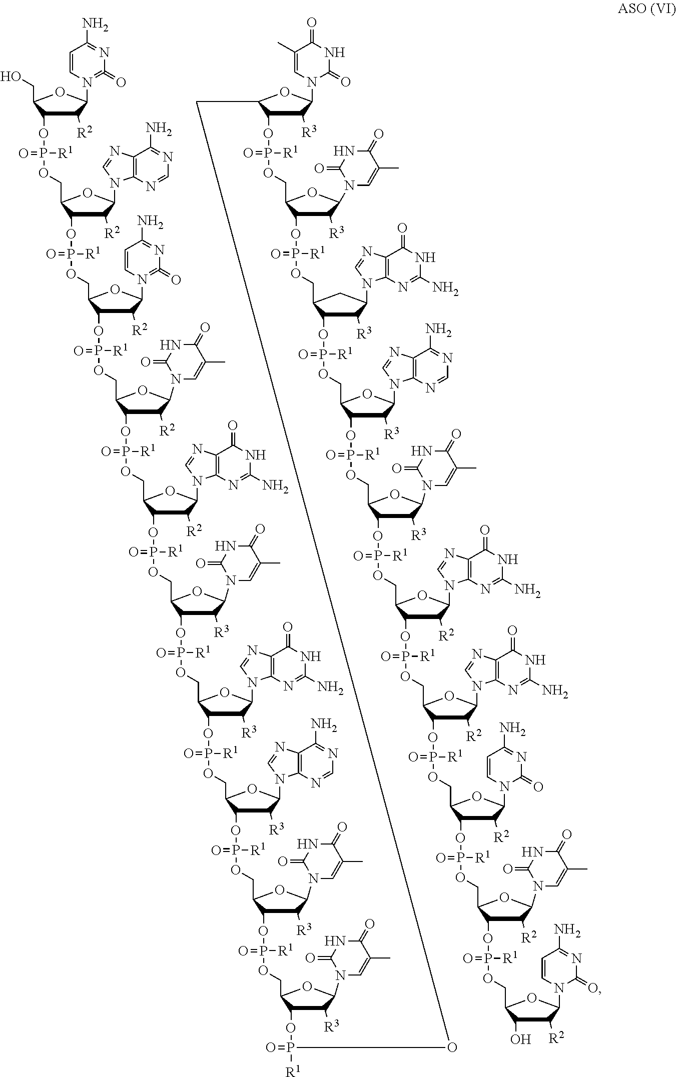

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and each R³ is independently selected from the group consisting of hydrogen, halo, OHR
OCH₃, OCF₃, OCH₂CH₃, OCH₂CH₂OCH₃, and OCH₂CF₃.

One embodiment provides an oligomeric compound (formula ASO (VII)) according to the formula:

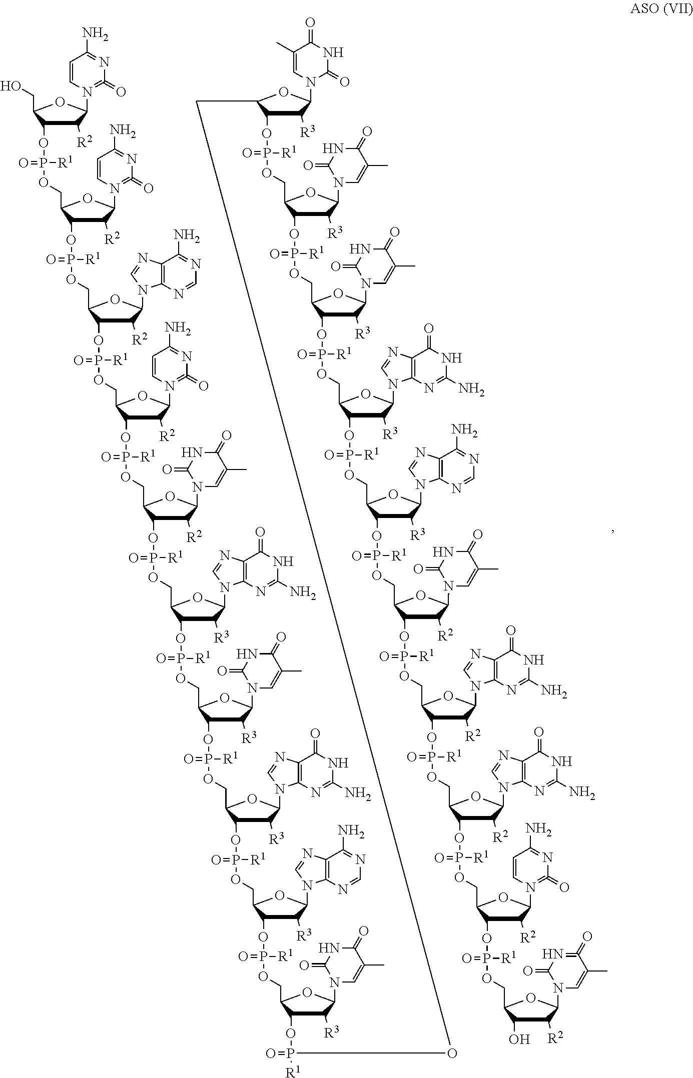

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OHI, OCH₃, OCF₃, OCH₂CH₃, OCH₂CH₂OCH₃, and OCH₂CF₃.

One embodiment provides an oligomeric compound (formula ASO (VIII)) according to the formula:

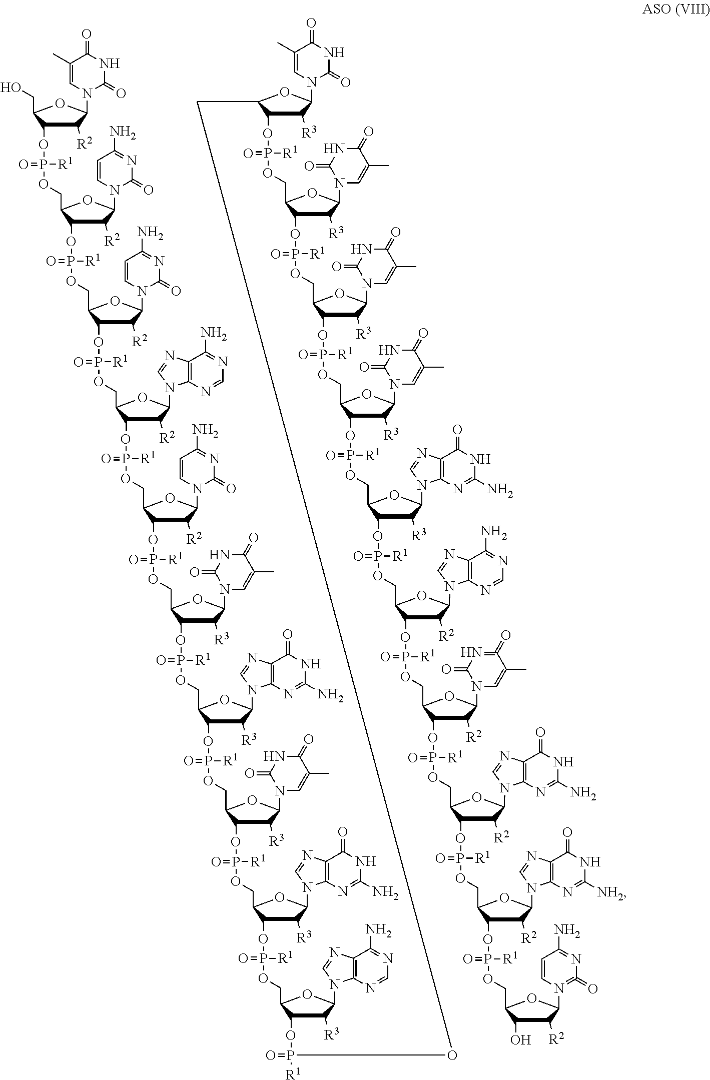

or a salt thereof, wherein:
each R¹ is independently selected from the group consisting of OH and SH;
each R² is independently selected from the group consisting of hydrogen, halo, OCH₃, OCF₃, OCH₂CH₃, and OCH₂CH₂OCH₃, OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (IX)) according to the formula:

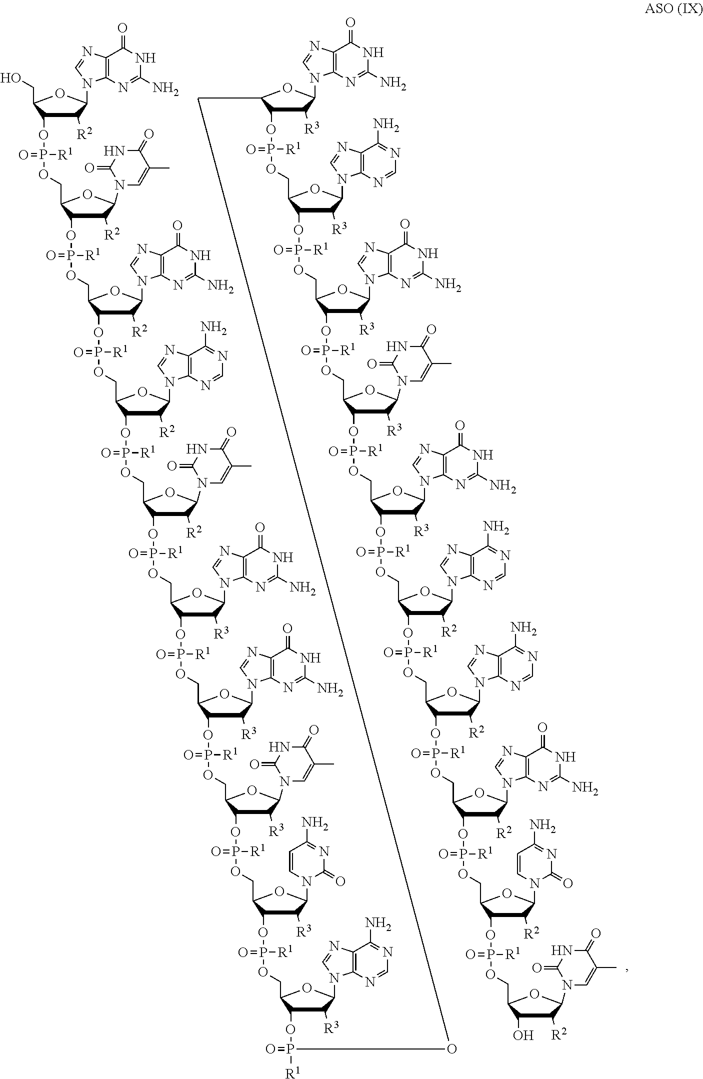

or a salt thereof, wherein:
each R^{t} is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃ and
each R is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (X)) according to the formula:

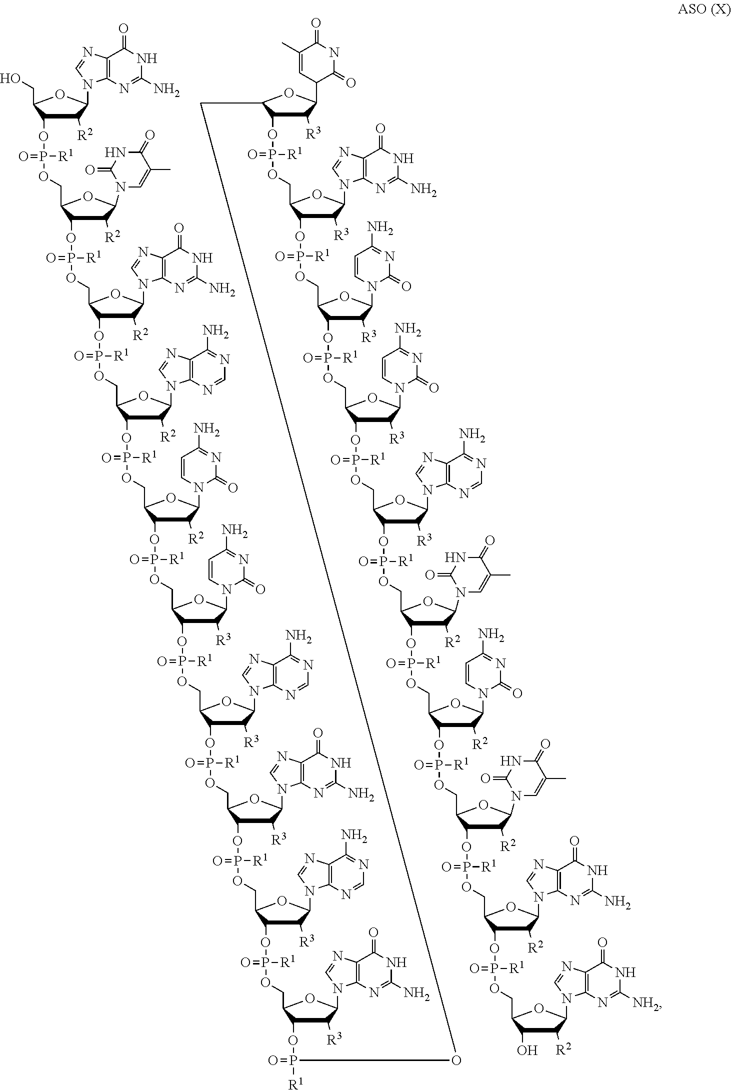

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (XI)) according to the formula:

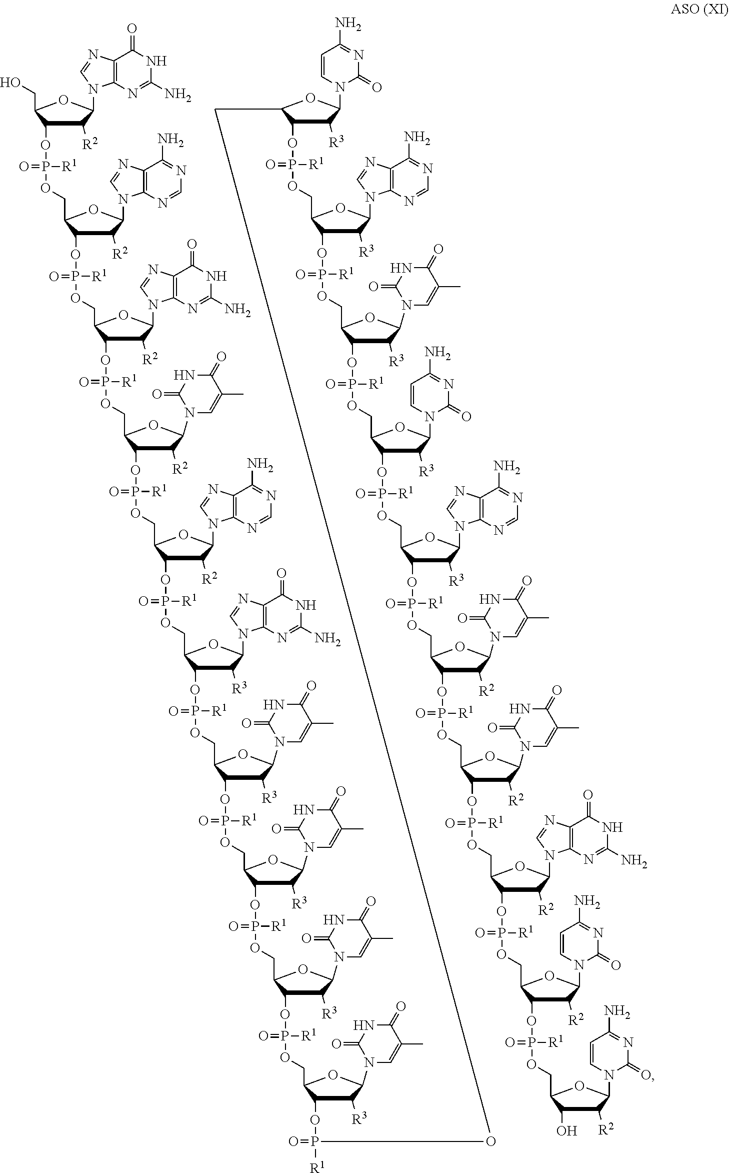

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (XII)) according to the formula:

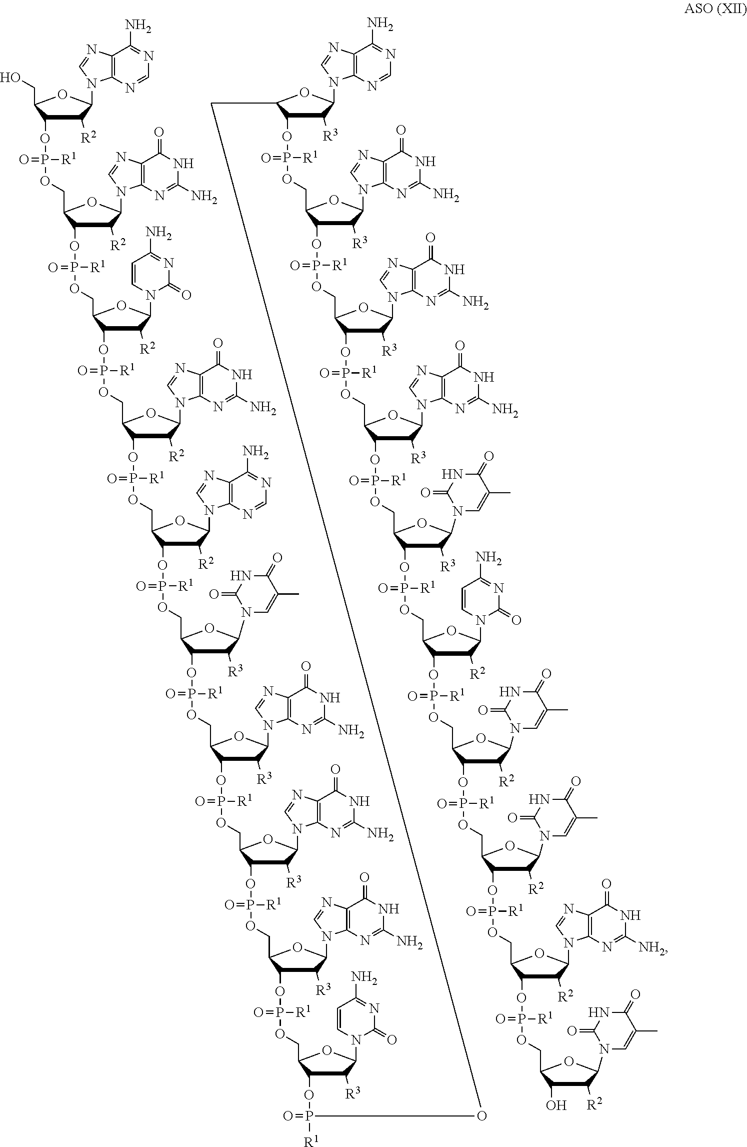

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and each R³ is independently selected from the group consisting of hydrogen, halo, OH,
OCH₃, OCF₃, OCH₂CH₃, OCH₂CH₂OCH₃, and OCH₂CF₃.

One embodiment provides an oligomeric compound (formula ASO (XIII)) according to the formula:

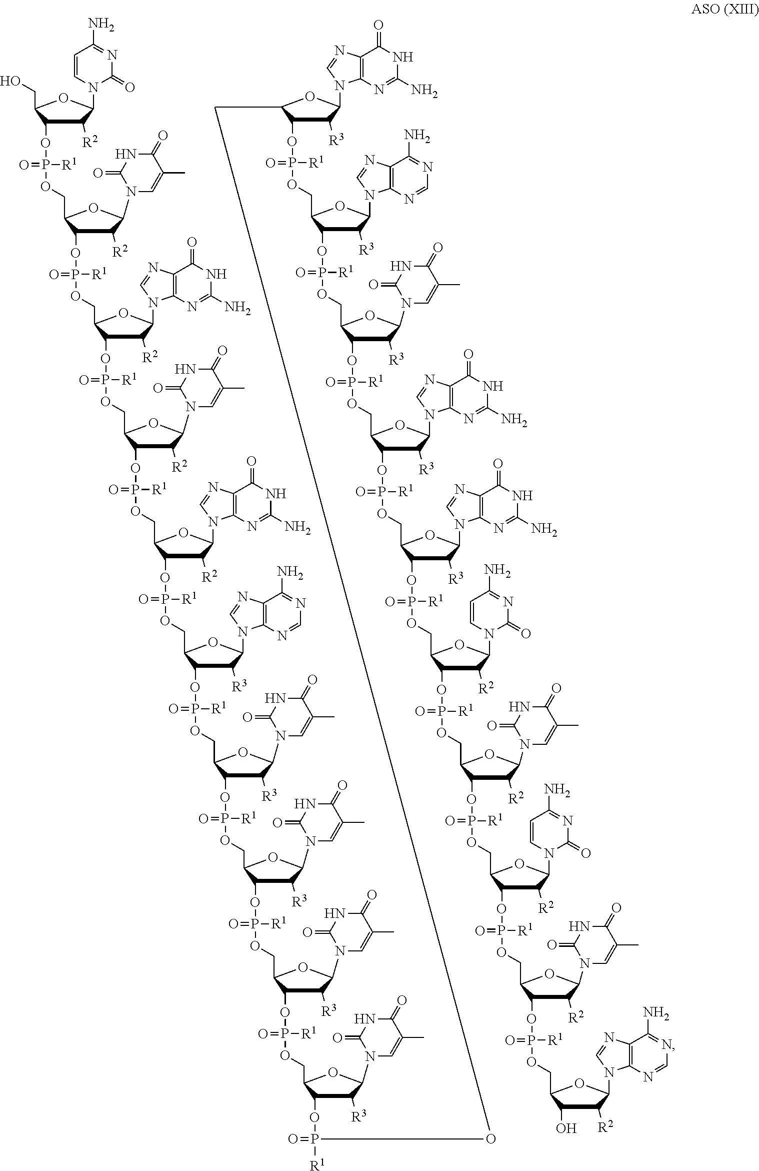

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (XIV)) according to the formula:

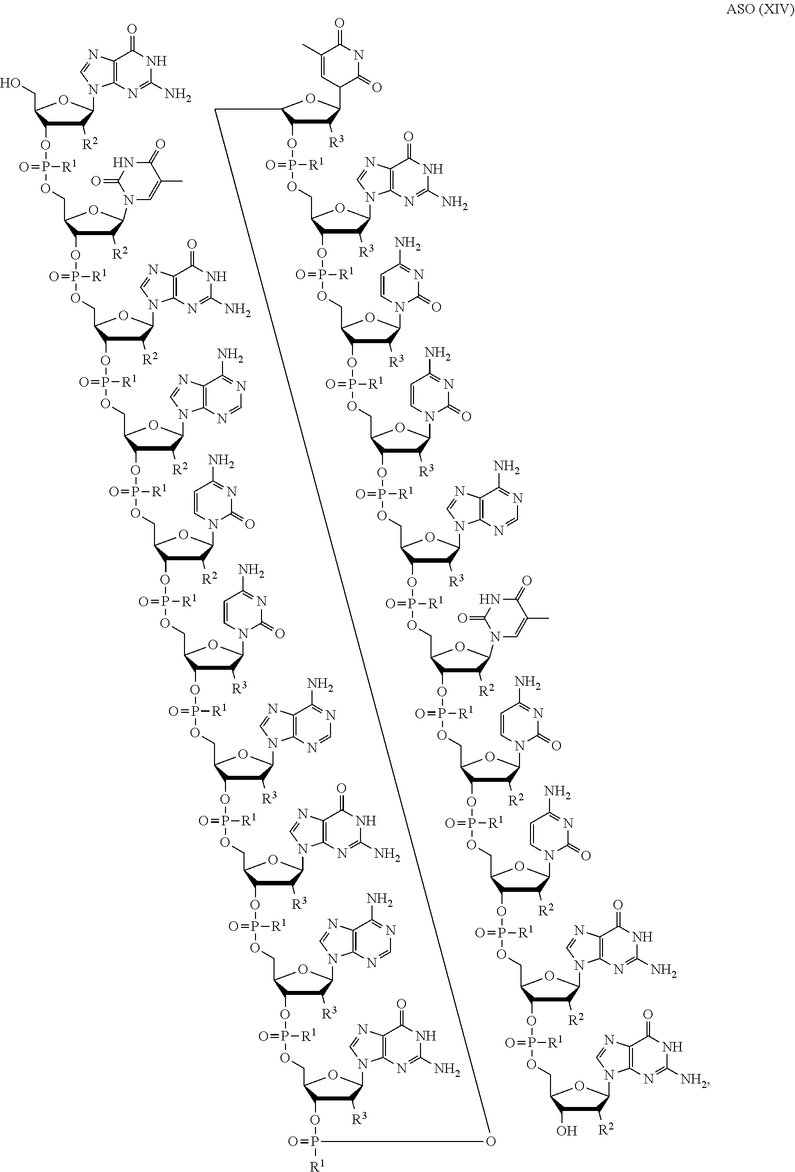

or a salt thereof, wherein: each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (XV)) according to the formula:

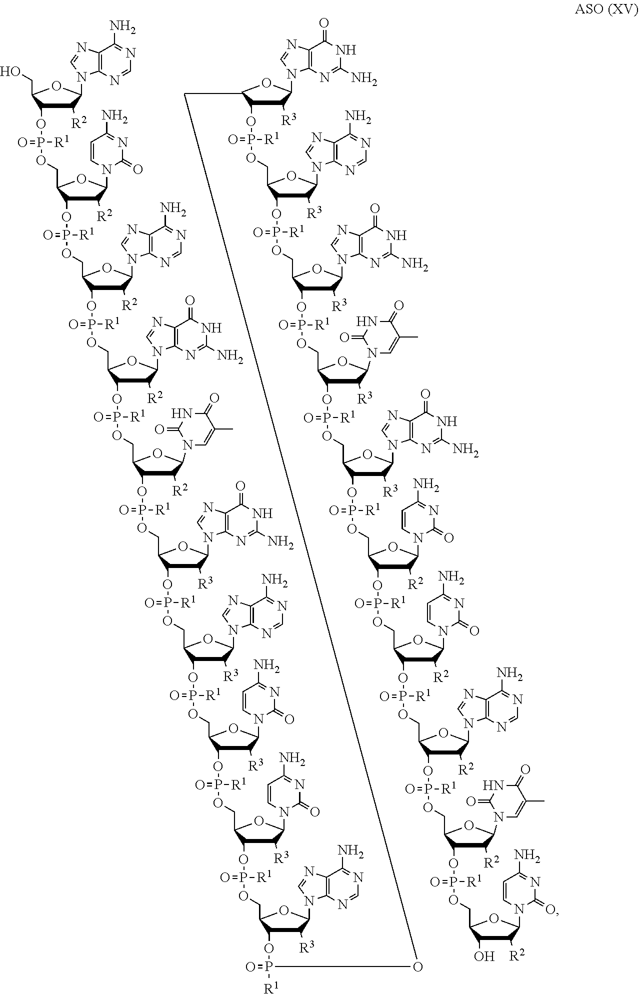

or a salt thereof, wherein:
each R¹ is independently selected from the group consisting of OH and SH;
each R² is independently selected from the group consisting of hydrogen, halo, OC, OCF₃, OCH₂CH₃, OCH₂CH₂OCH₃, and OCH₂CF₃; and
each R³ is independently selected from the group consisting of hydrogen, halo. OH, OCH₃OCF₃, OCH₂CH₃, OCH₂CH₂OCH₃, and OCH₂CF₃.

One embodiment provides an oligomeric compound (formula ASO (XVI)) according to the formula:

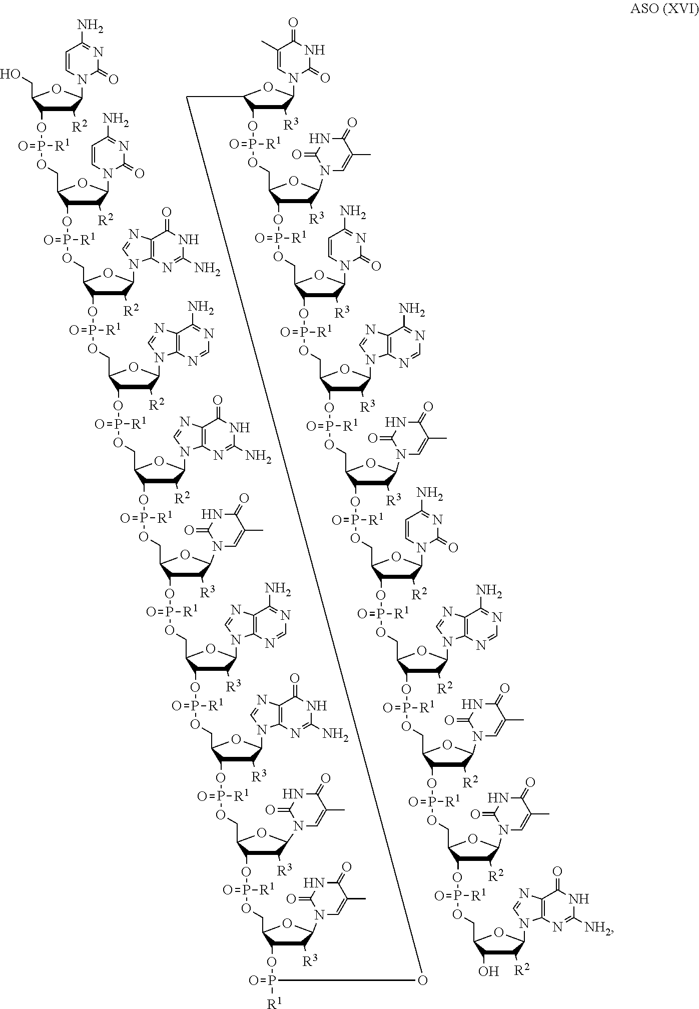

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (XVII)) according to the formula:

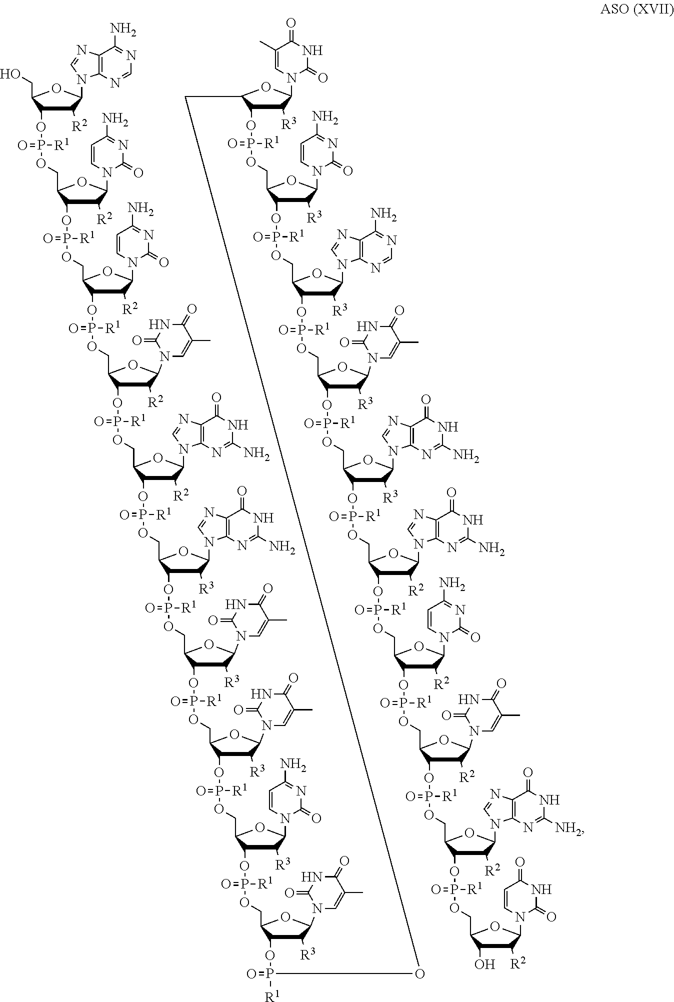

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen halo, OH, OCH₃, OCF₃, OCH₂CH₃, OCH₂CH₂C₃, and OCH₂CF₃.

One embodiment provides an oligomeric compound (formula ASO (XVIII)) according to the formula:

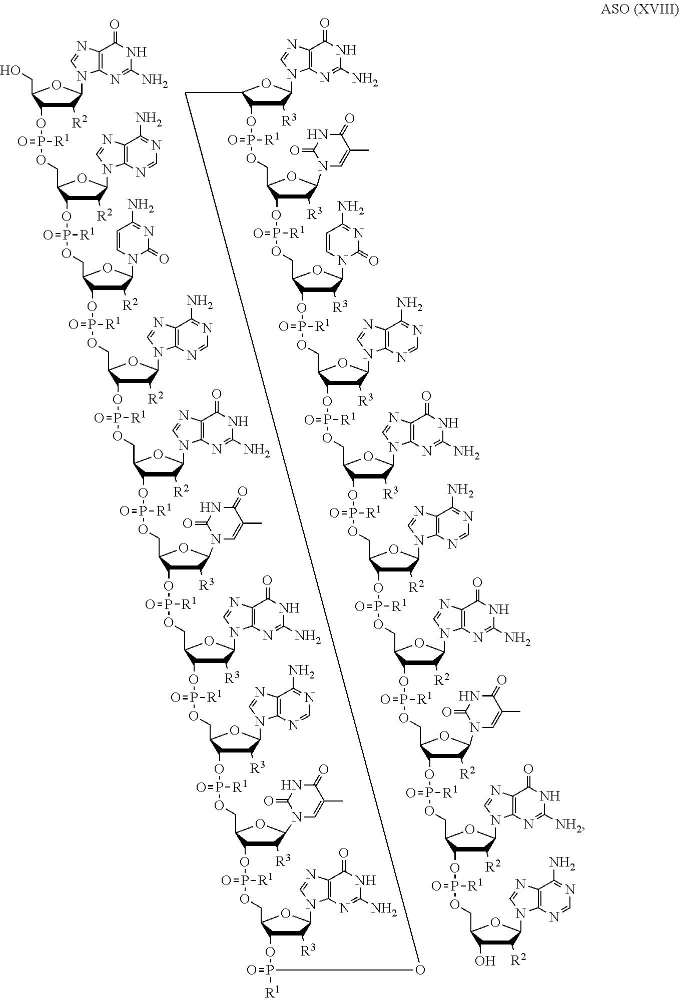

or a salt thereof, wherein:
each R¹ is independently selected from the group consisting of OH and SH;
each R² is independently selected from the group consisting of hydrogen, halo, OCH₃,
OCF₃, OCH₂CH₃, OCH₂CH₂OCH₃, and OCHCF₃ and
each R³ is independently selected from the group consisting of hydrogen, halo, OH, OCH₃, OCF₃, OCH₂CH₃, OCH₂CH₂OCH₃, and OCH₂CF₃.

One embodiment provides an oligomeric compound (formula ASO (XIX)) according to the formula:

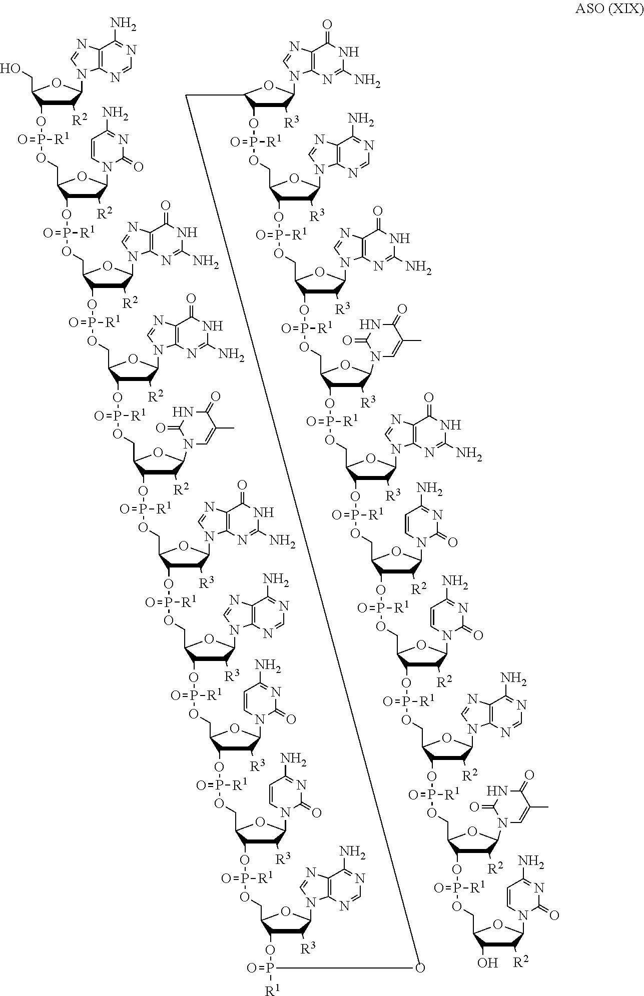

or a salt thereof, wherein:
each R¹ is independently selected from the group consisting of OH and SF₁;
each R² is independently selected from the group consisting of hydrogen, halo, OCH₃, OCF₃, OCH₂CH₃, OCH₂CH₂OCH₃, and OCH₂CF₃; and
each R³ is independently selected from the group consisting of hydrogen, halo, OH, OCH₃, OCF₃, OCH₂CH₃, OCH₂CH₂OCH₃, and OCH₂CF₃.

In certain embodiments, for the oligomeric compounds described herein, R³ is hydrogen. In some embodiments R² can be OCH₂CH₃ or OCH₃. In some embodiments R² is OCH₂CH₂OCH₃. In some embodiments R¹ is SH₄. In some embodiments, an oligomeric compound described herein, or a salt thereof, comprises an oligomeric duplex. In some embodiments, an oligomeric compound described herein, or a salt thereof or the oligomeric duplex comprises an antisense compound.

One embodiment provides an oligomeric compound (formula ASO (1)) according to the

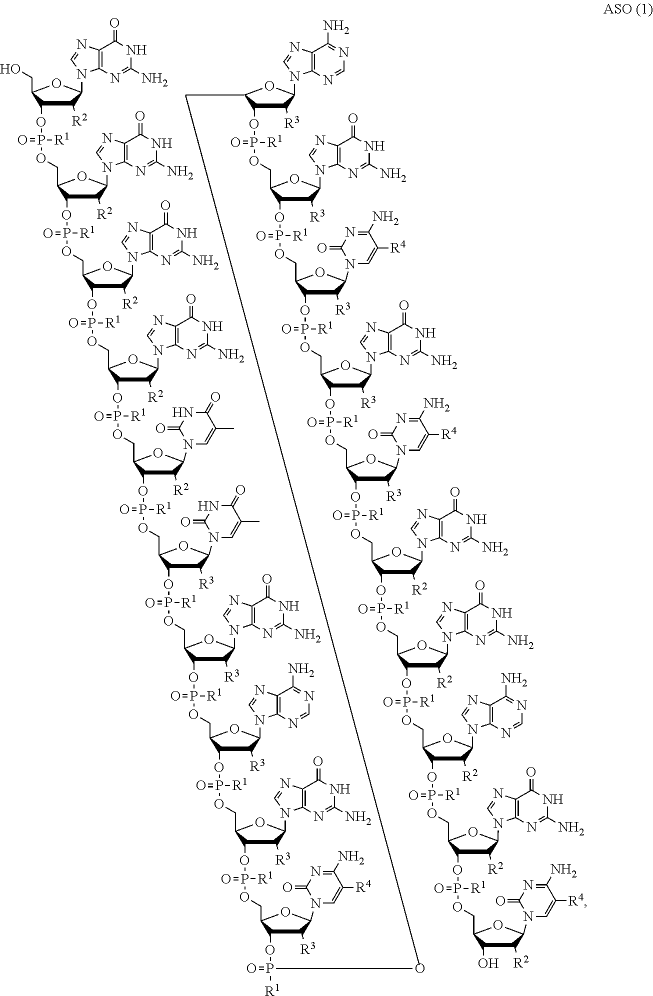

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCHF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (2)) according to the following formula:

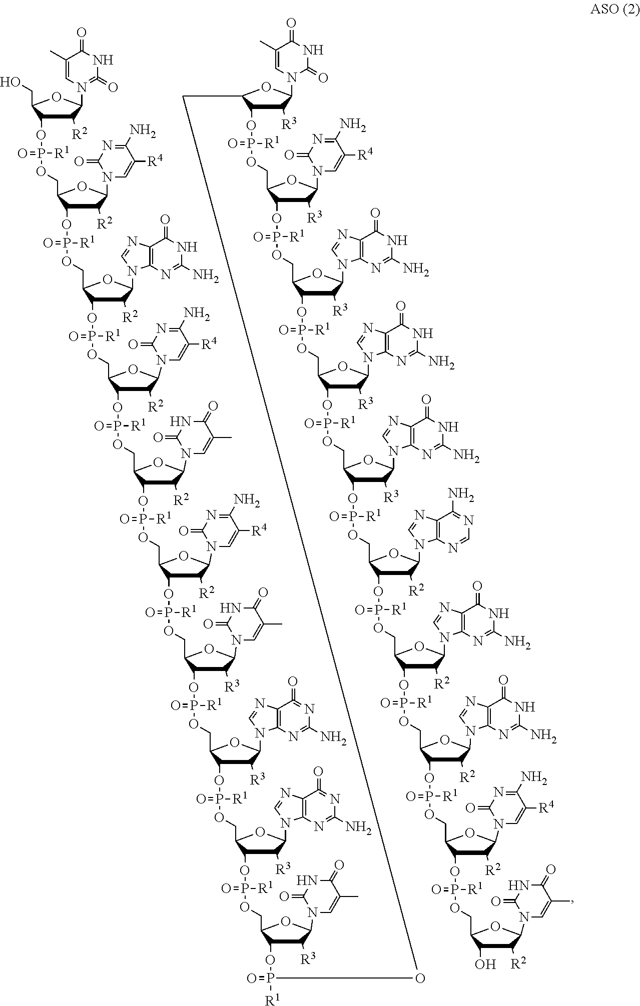

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (3)) according to the following formula:

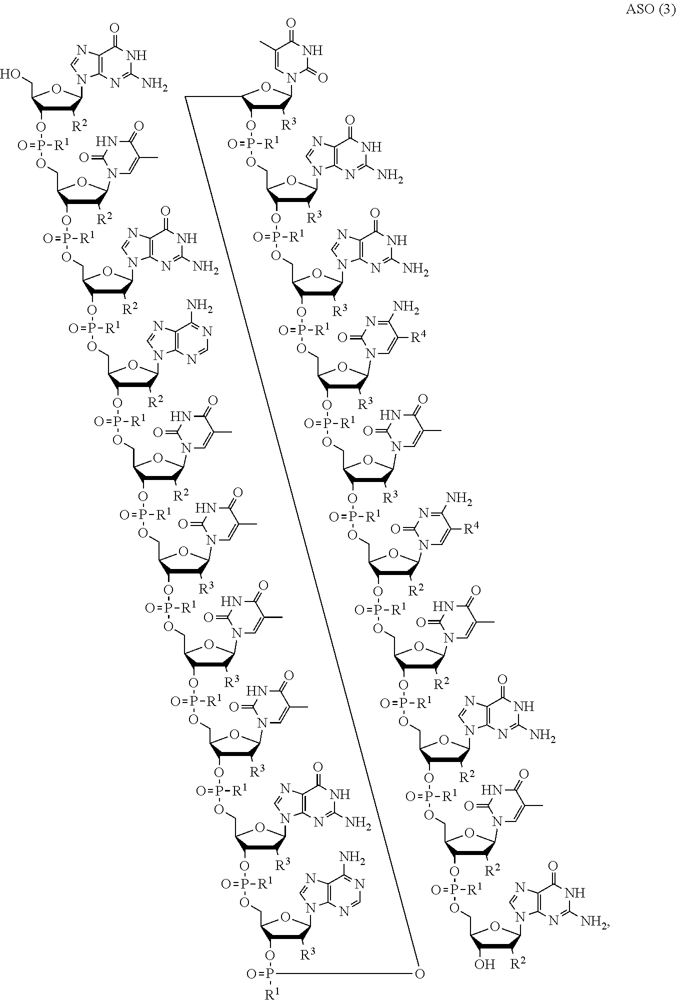

or a salt thereof, wherein:
each R¹ is independently selected from the group consisting of OH and SH;
each R² is independently selected from the group consisting of hydrogen, halo, OCH₃, OCF₃, OCH₂CH₃, OH₂CH₂OCH₃, and OCH₂CF₃;
each R³ is independently selected from the group consisting of hydrogen, halo, OH, OCH₃, OCHF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (4)) according to the formula:

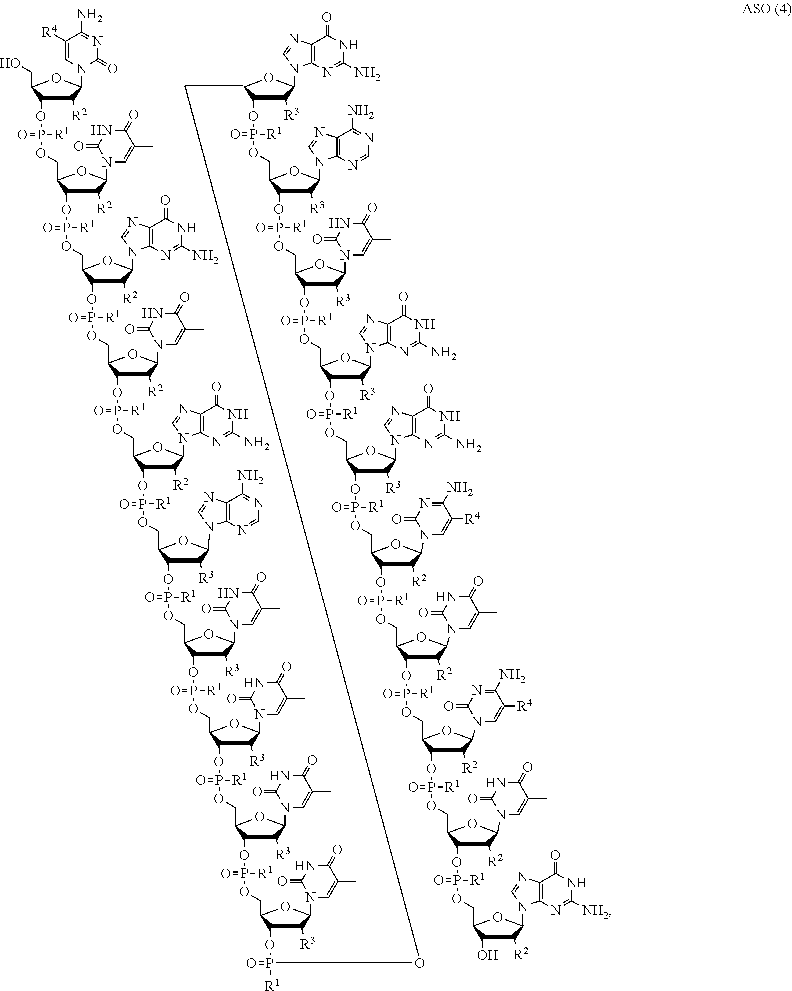

or a salt thereof, wherein:
each R¹ is independently selected from the group consisting of OH and SH;
each R² is independently selected from the group consisting of hydrogen, halo, OCH₃, OCF₃, OCH₂CH₃, OCH₂C₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (5)) according to the formula:

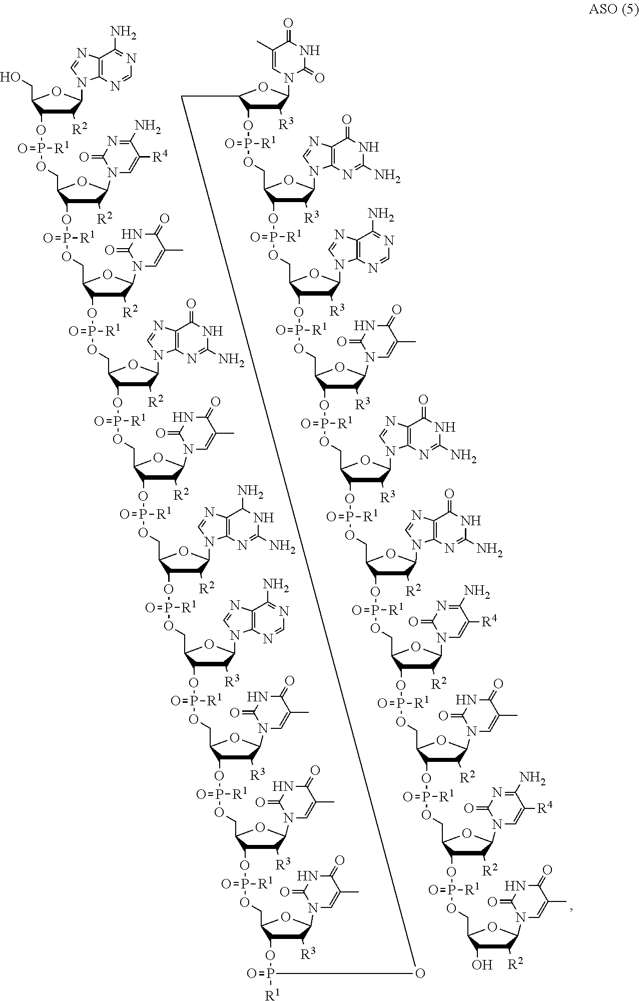

or a salt thereof, wherein:
each R¹ is independently selected from the group consisting of OH and SH;
each R² is independently selected from the group consisting of hydrogen, halo,
OCH₃, OCF₃, OCH₂C1H₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (6)) according to the formula:

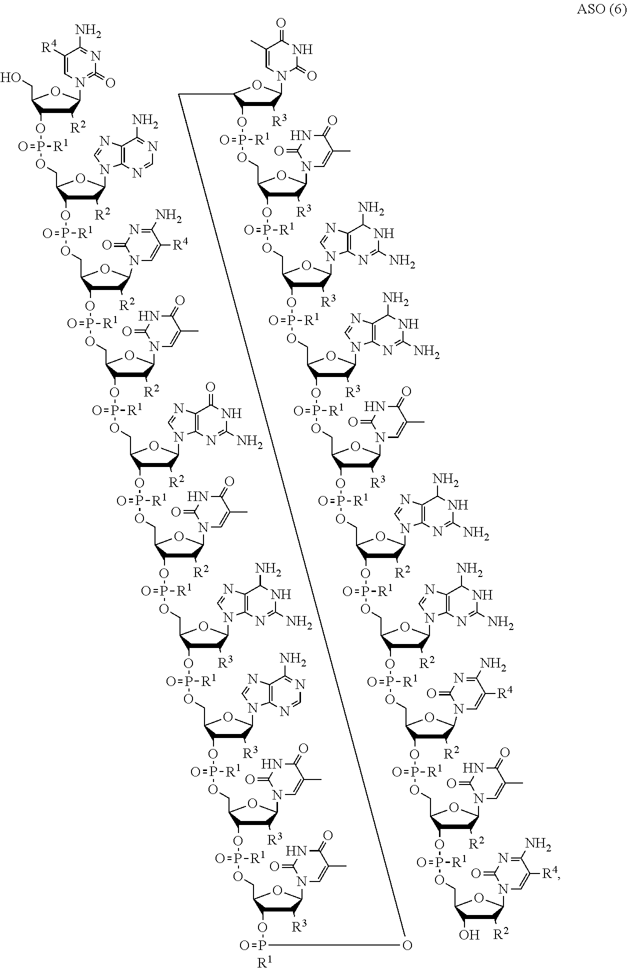

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (7)) according to the formula:

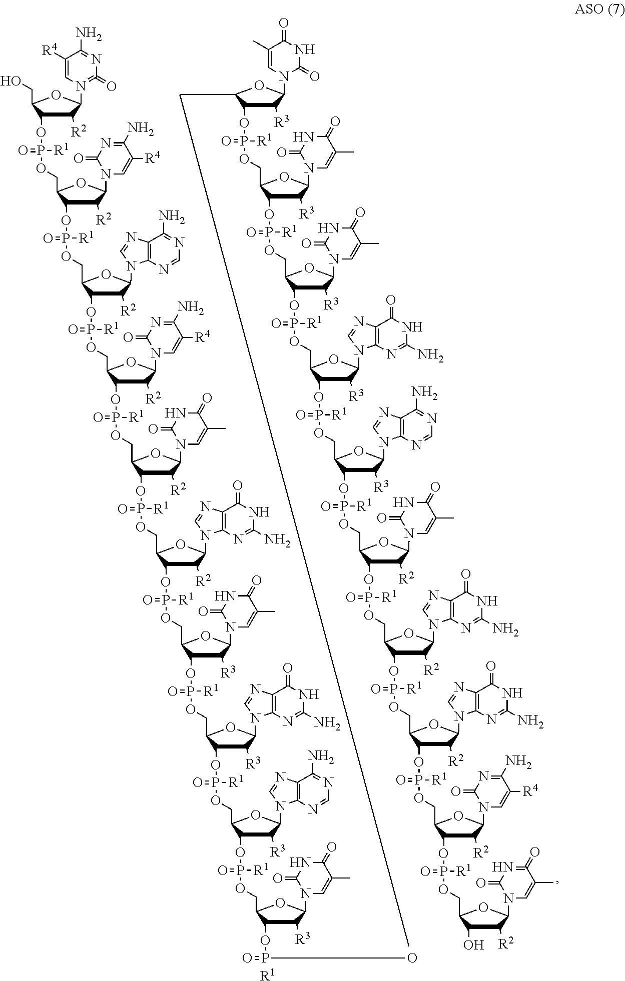

or a salt thereof, wherein:
each R¹ is independently selected from the group consisting of OH and SH;
each R² is independently selected from the group consisting of hydrogen, halo OCH₃, OCF₃, OCH₂CH₃, OCH₂CH₂OCH₃, and OCH₂CF₃;
each R³ is independently selected from the group consisting of hydrogen, halo, OHR OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (8)) according to the formula:

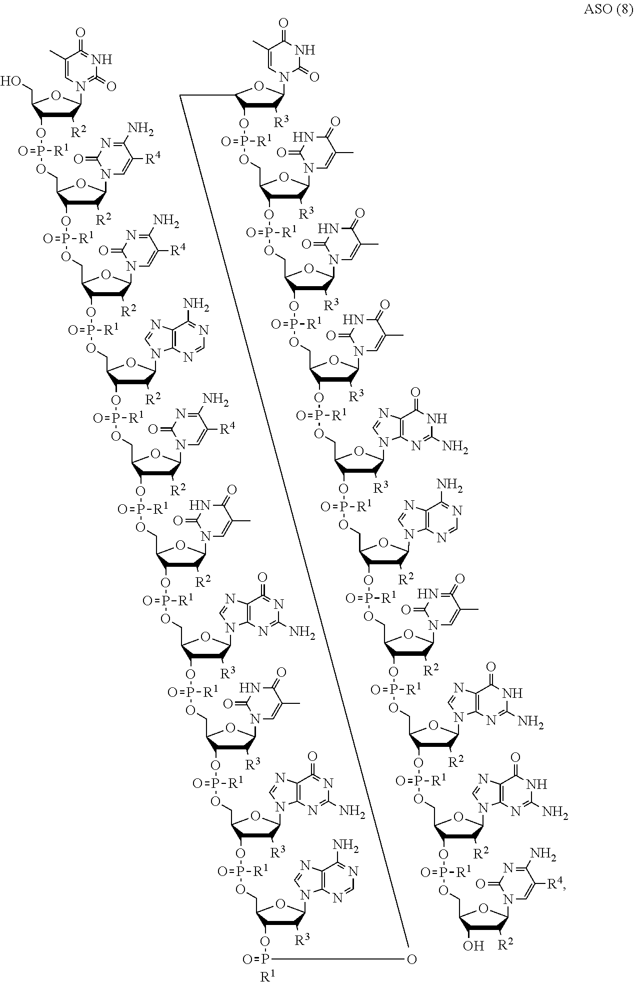

or a salt thereof, wherein:
each R¹ is independently selected from the group consisting of OH and SH;
each R² is independently selected from the group consisting of hydrogen, halo, OCH₃, OCF₃, OCH₂CH₃, and OCH₂CH₂OCH₃, OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (9)) according to the formula:

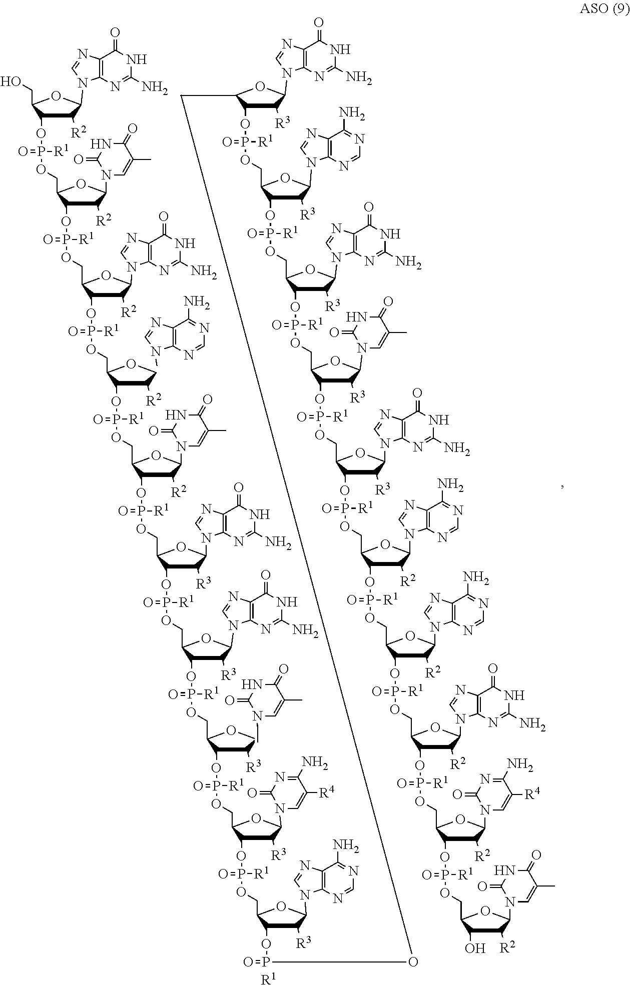

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (10)) according to the formula:

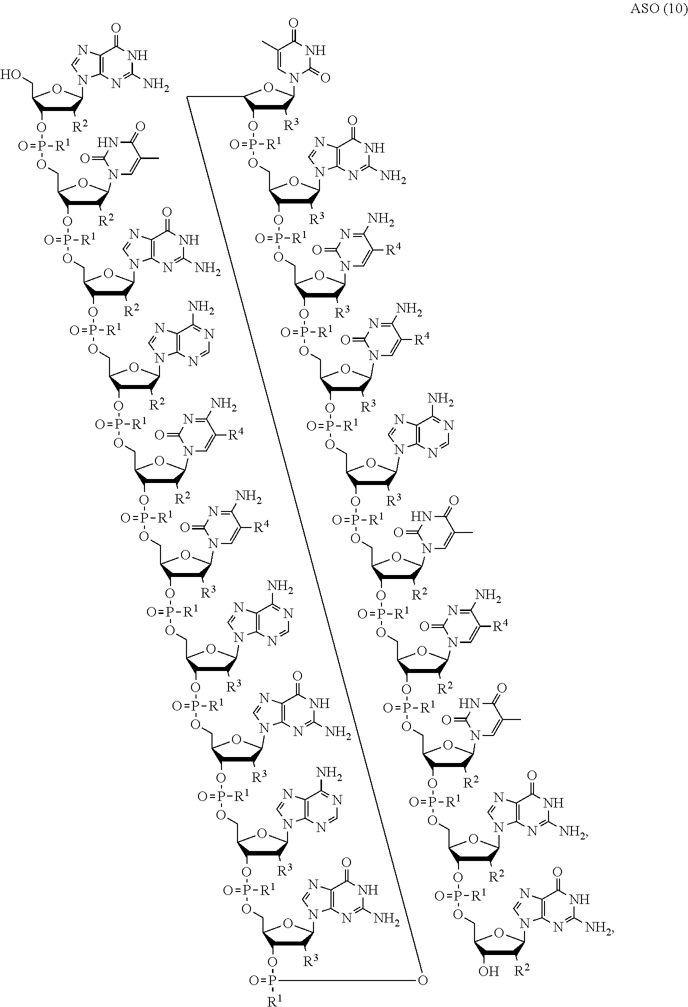

each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₁₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (11)) according to the formula:

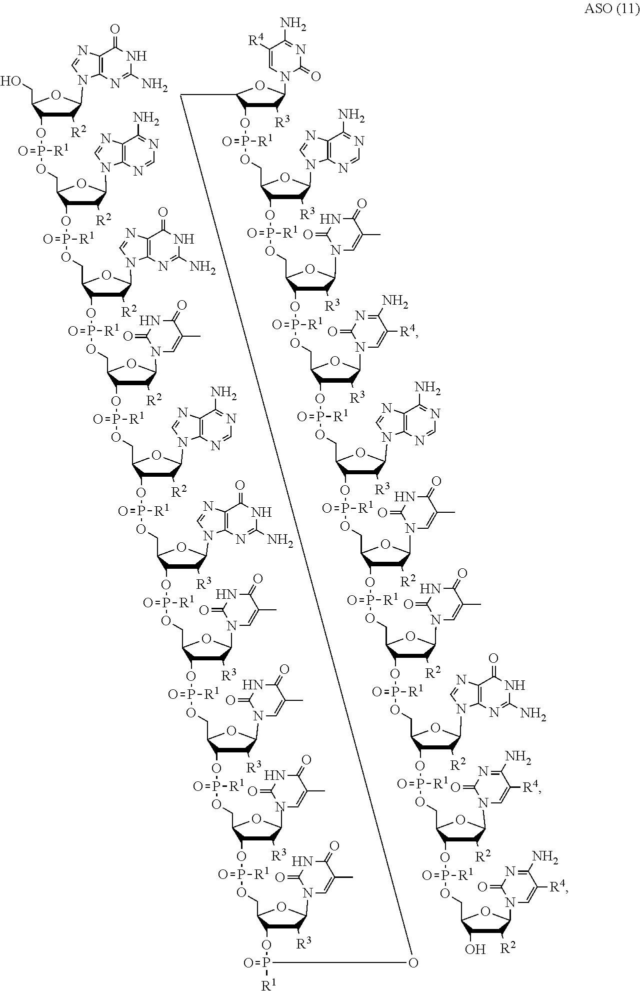

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (12)) according to the formula:

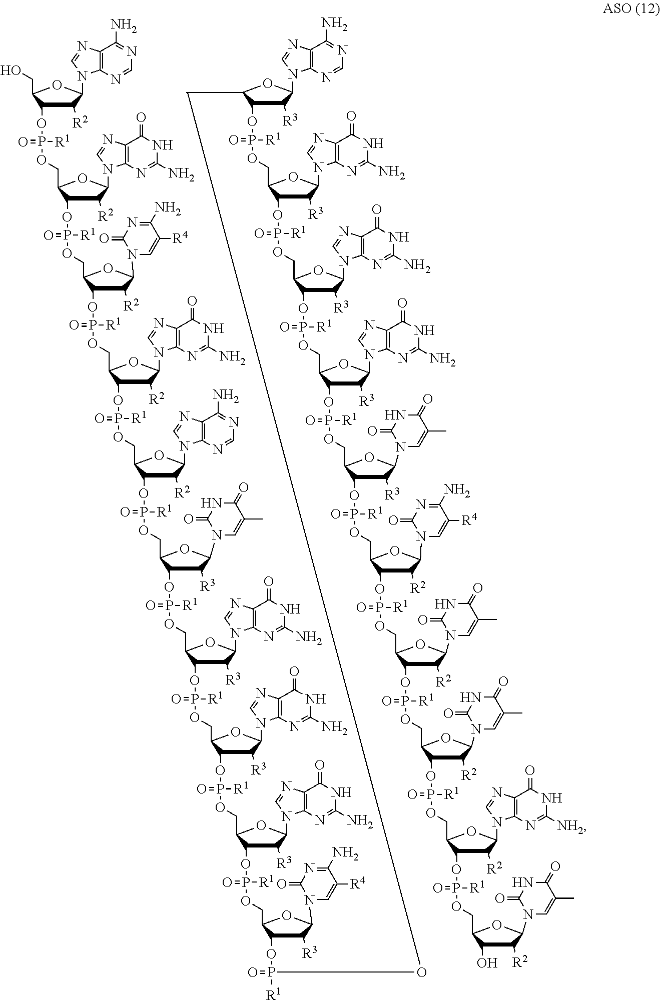

or a salt thereof, wherein:
each R¹ is independently selected from the group consisting of OH and SH;
each R² is independently selected from the group consisting of hydrogen, halo, OCH₃, OCH₃, OCF₃, OCH₂CH₃, OCH₂CH₂OH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (13)) according to the formula:

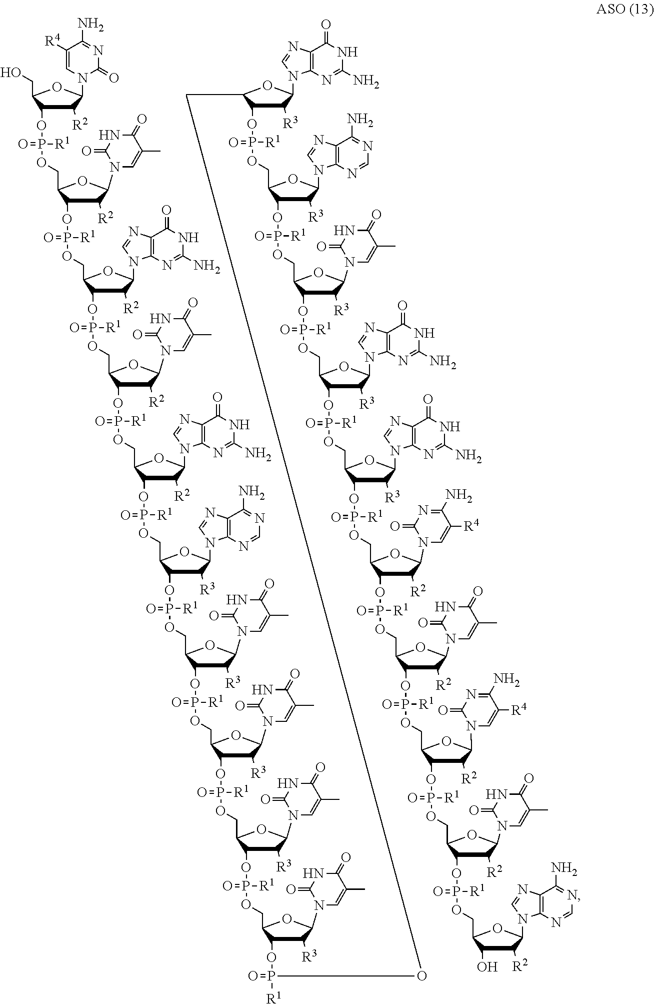

or a salt thereof, wherein:
each R³ is independently selected from the group consisting of OH and SH;
each R² is independently selected from the group consisting of hydrogen, halo, OCH₃, OCF₃, OCF₃OCH₂CH₃, OCH₂CH₂CH₃, and OCH₂CF₃;
each R³ is independently selected from the group consisting of hydrogen, halo, OH. OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (14)) according to the formula:

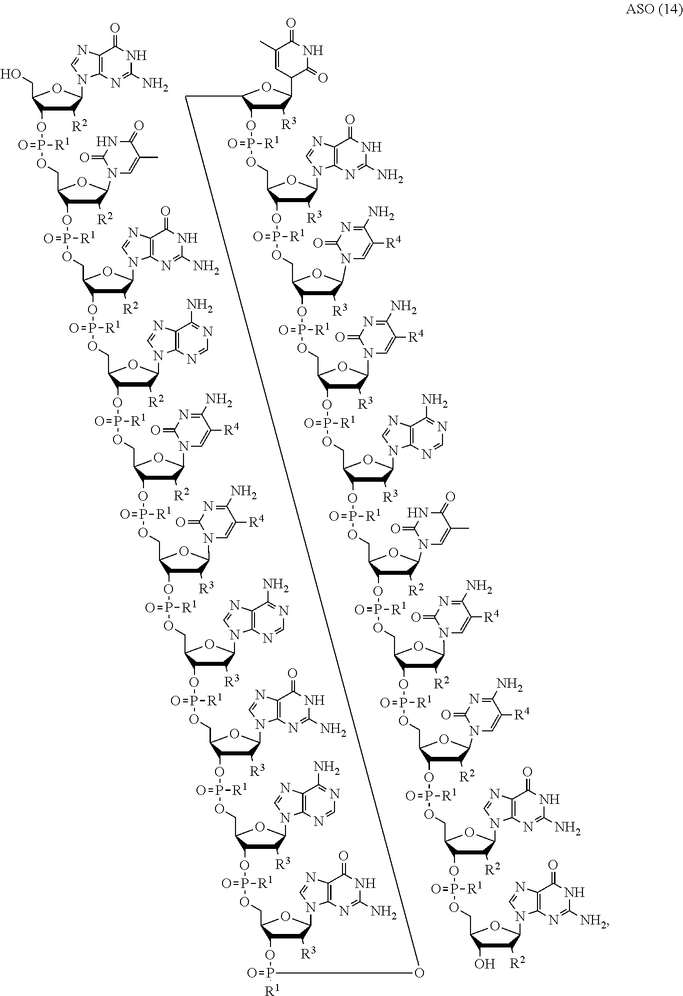

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (15)) according to the formula:

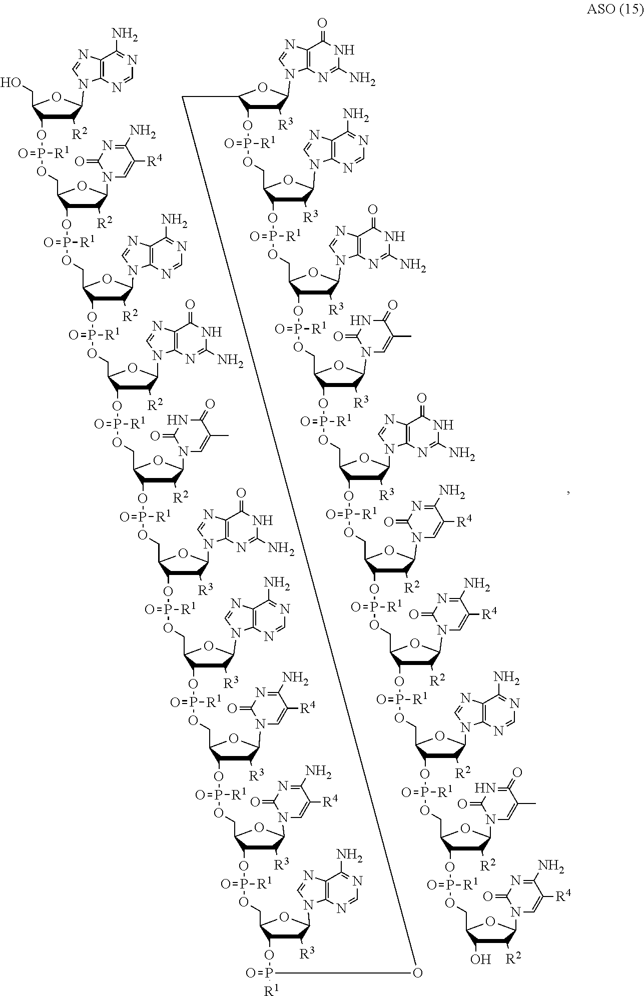

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (16)) according to the formula:

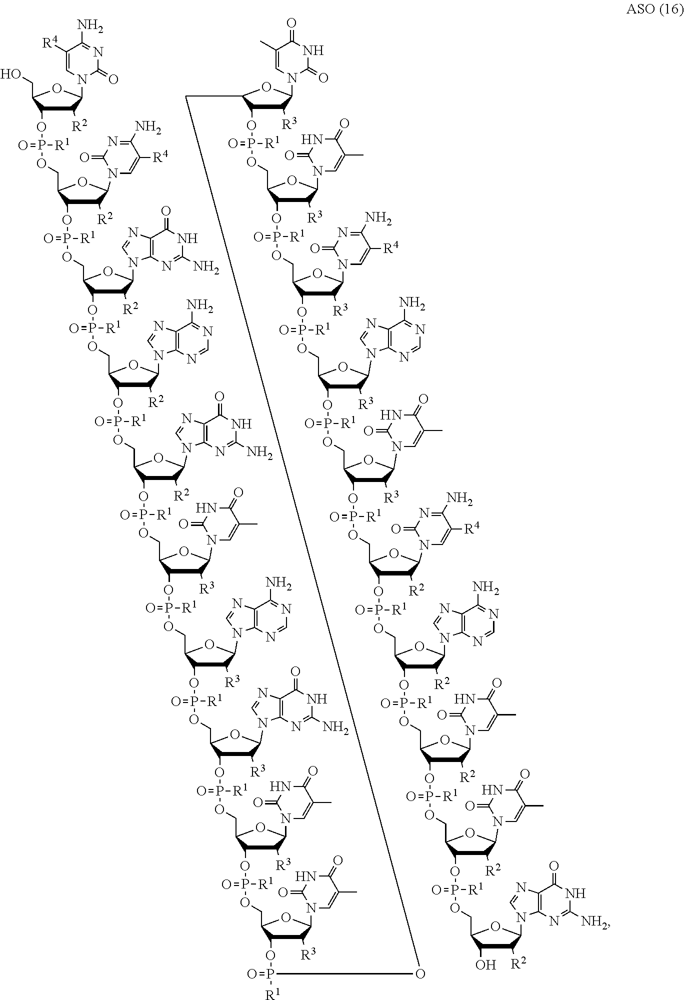

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (17)) according to the formula:

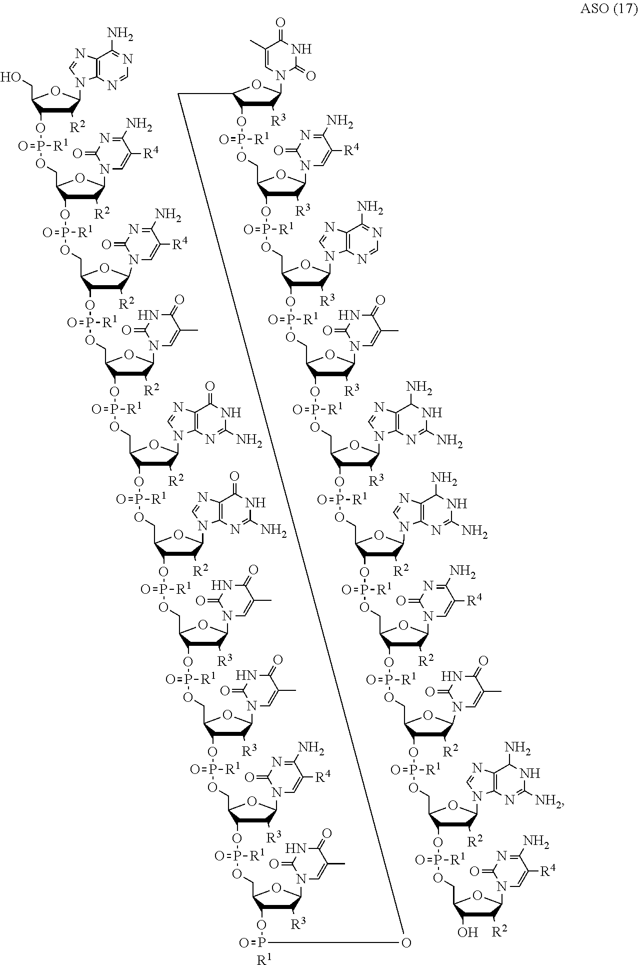

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (18)) according to the formula:

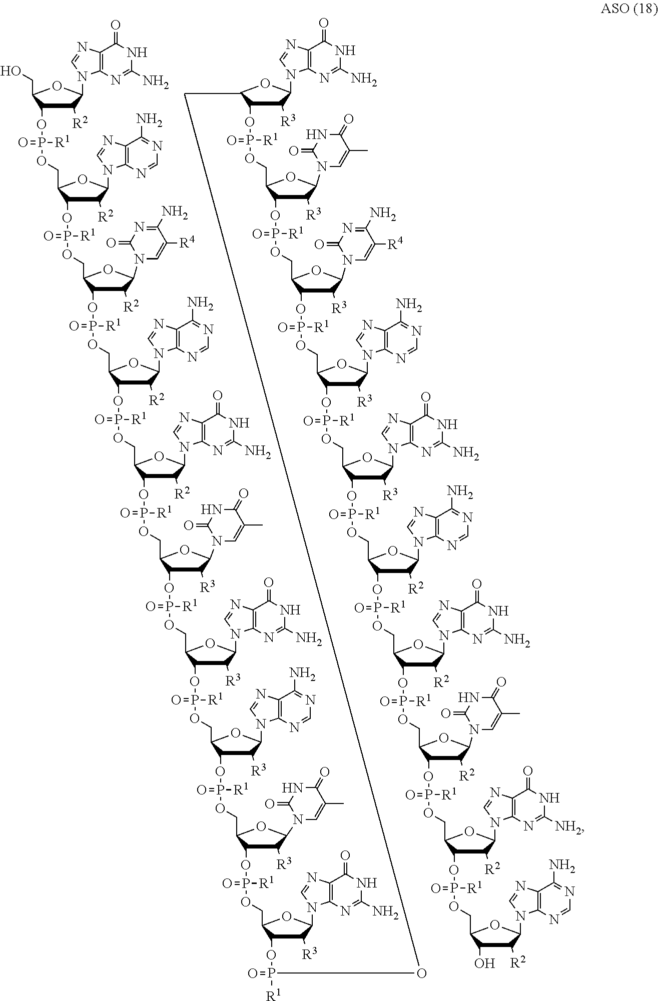

or a salt thereof, wherein:
each R¹ is independently selected from the group consisting of 01H and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, 01H, OCH₃, OCF₃, OCH₂CH₃, OCH₂CH₂OCH₃, and OCH₂CF₃; and
each R⁴ is independently selected from the group consisting of hydrogen and CH₃.

One embodiment provides an oligomeric compound (formula ASO (19)) according to the formula:

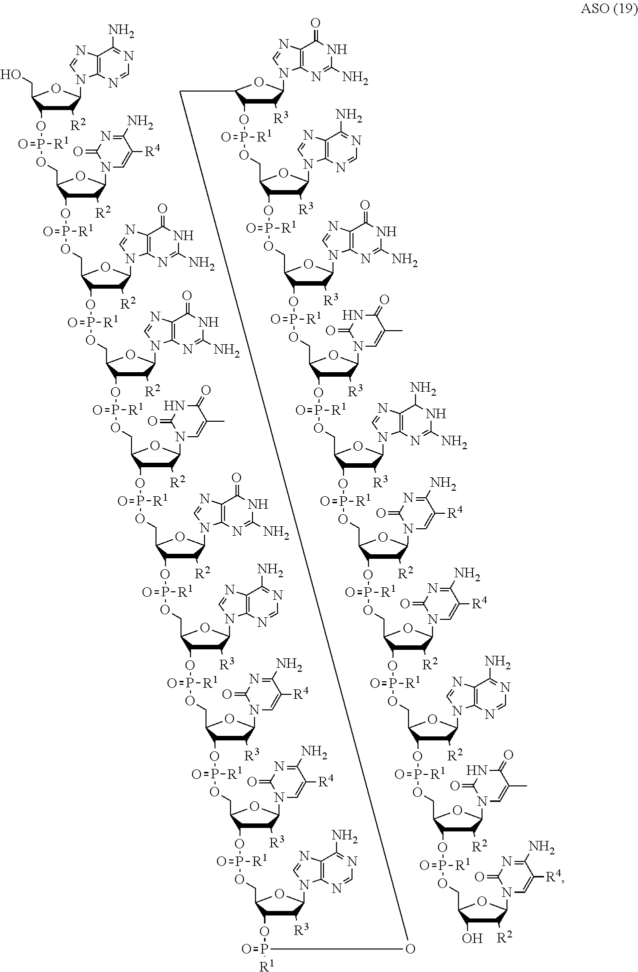

or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

In certain embodiments, for the oligomeric compounds described herein, R³ is hydrogen. In some embodiments R² can be OCH₂CH₃ or OCH₃. In some embodiments R² is OCH₂CH₂OCH₃. In some embodiments R¹ is SH. In certain embodiments, at least one R⁴ is CH₃. In some embodiments, each R⁴ is CH₃. In certain embodiments each R⁴ is hydrogen. In some embodiments an oligomeric compound described herein or a salt thereof, comprises an oligomeric duplex. In some embodiments, an oligomeric compound described herein, or a salt thereof or the oligomeric duplex comprises an antisense compound.

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that it is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a RUNX1 nucleic acid is 12 to 30 subunits in length. In other words, such antisense compounds are from 12 to 30 linked subunits. In certain embodiments, the antisense compound is 5 to 80, 8 to 50, 10 to 30, 16 to 24, 18 to 22, or 20 linked subunits. In certain embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 4344, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments, the antisense compounds are 18 subunits in length. In some embodiments, the antisense compounds are 19 subunits in length. In some embodiments, the antisense compounds are 20 subunits in length. In some embodiments, the antisense compounds are 21 subunits in length. In some embodiments, the antisense compounds are 22 subunits in length. In some embodiments, the antisense compounds are 23 subunits in length. In some embodiments the antisense compound is an antisense oligonucleotide, and the linked subunits are nucleosides.

In certain embodiments antisense oligonucleotides targeted to a RUNX1 nucleic acid may be shortened or truncated. For example, a single subunit may be deleted from the 5′ end (5′ truncation), or alternatively from the 3′ end (3′ truncation). A shortened or truncated antisense compound targeted to a RUTNX1 nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, see Woolf et al. (Proc. Natl. Acad. Sci. USA 89:7305-7309, 1992), Gautschi et al (J. Natl. Cancer Inst. 93:463-471, March 2001), and Maher and Dolnick (Nuc. Acid. Res. 16:3341-3358, 1988).

### Antisense Compound Motifs

In certain embodiments, antisense compounds targeted to a RUNX1 nucleic acid have chemically modified subunits arranged in patterns, or motifs, to confer to the antisense compounds' properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease PKNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)—O-2′ bridge, where n=1 or n=2 and 4′-CH₂—O—CH₂-2′). Preferably, each distinct region comprises uniform sugar moieties. The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers described herein include, but are not limited to, for example 5-10-5, 5-10-4, 4-10-4, 4-10-3, 3-10-3, 2-10-2, 5-9-5, 5-9-4, 4-9-5, 5-8-5, 5-8-4, 4-8-5, 5-7-5, 4-7-5, 5-7-4, or 4-7-4. In some embodiments the gapmers described herein are 5-10-5. In some embodiments the gapmers described herein are 4-10-5. In some embodiments the gapmers described herein are 5-10-4. In some embodiments the gapmers described herein are 4-10-4.

In certain embodiments, the antisense compound has a “wingmer” motif, having a wing-gap or gap-wing configuration, i.e. an X-Y or Y-Z configuration as described above for the gapmer configuration. Thus, wingmer configurations described herein include, but are not limited to, for example 5-10, 8-4, 4-12, 12-4, 3-14, 16-2, 18-1, 10-3, 2-10, 1-10, 8-2, 2-13, 5-13, 5-8, or 6-8.

In certain embodiments, an antisense compound targeted to a RUNX1 nucleic acid has a gap-narrowed motif. In certain embodiments, a gap-narrowed antisense oligonucleotide targeted to a RUNX1 nucleic acid has a gap segment of 9, 8, 7, or 6 2′-deoxynucleotides positioned immediately adjacent to and between wing segments of 5, 4, 3, 2, or 1 chemically modified nucleosides. In certain embodiments, the chemical modification comprises a bicyclic sugar. In certain embodiments, the bicyclic sugar comprises a 4′ to 2′ bridge selected from among: 4′-(CH₂)ₙ—O-2′ bridge, wherein n is 1 or 2; and 4′-CH₂—O—CH₂-2′. In certain embodiments, the bicyclic sugar comprises a 4′-CH(CH₃)—O-2′ bridge. In certain embodiments, the chemical modification comprises a non-bicyclic 2′-modified sugar moiety. In certain embodiments, the non-bicyclic 2′-modified sugar moiety comprises a 2′-O-methylethyl group or a 2′-O-methyl group.

### Target Nucleic Acids, Target Regions, and Nucleotide Sequences

Nucleotide sequences that encode RUNX1 can include, without limitation, the following: SEQ ID NO: 1-SEQ ID NO: 19.

It is understood that antisense compounds complementary to any of SEQ ID NO: 1-SEQ ID NO: 19 can comprise, independently, one or more modifications to a sugar moiety, an internucleoside linkage, or a nucleobase.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for RUNX1 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5‘ U’JR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within a target region. In certain embodiments, reductions in RUNX1 mRNA levels are indicative of inhibition of RUNX1 expression. Reductions in levels of a RUNX1 protein are also indicative of inhibition of target mRNA expression. Reduction in the presence of expanded RUNX1 RNA foci are indicative of inhibition of RUNX1 expression. Further, phenotypic changes are indicative of inhibition of RUNX1 expression. For example, improved memory and potentially additional cognitive attributes in individuals with DS may be indicative of inhibition of RUNX1 expression.

### Hybridization

In some embodiments, hybridization occurs between an antisense compound disclosed herein and a RUNX1 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a RUNX1 nucleic acid, a target region, target segment, or specified portion thereof. In some embodiments, the RUNX1 nucleic acid is RUNX1 mRNA.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a RUNX1 nucleic acid, a target region, target segment, or specified portion thereof). In some embodiments, the RUNX1 nucleic acid is RUNX1 mRNA.

Non-complementary nucleobases between an antisense compound and a RUNX1 nucleic acid can be tolerated provided that the antisense compound remains able to specifically hybridize to a target nucleic acid. Moreover, an antisense compound can hybridize over one or more segments of a RUTNX1 nucleic acid such that intervening or adjacent segments are not involved in the hybridization event (e.g., a loop structure, mismatch or hairpin structure). In some embodiments., the RUNX1 nucleic acid is RUNX1 mRNA.

In certain embodiments, the antisense compounds provided herein, or a specified portion thereof, are, or are at least, 70%, 80%, 85% 86%, 87%, 88%, 89%, 90%, 91%, 92%, 93%, 94%, 95%, 96%, 97%, 98%, 99%, or 100% complementary to a RUNX1 nucleic acid, a target region, target segment, or specified portion thereof. Percent complementarity of an antisense compound with a target nucleic acid can be determined using routine methods. In some embodiments, the RUNX1 nucleic acid is RUNX1 mRNA.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol. 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a RUNX1 nucleic acid, or a target region, or a target segment or target sequence thereof. In some embodiments, the RUNX1 nucleic acid is RUNX1 mRNA. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 10, 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a RUNX1 mRNA, or specified portion thereof.

In certain embodiments, antisense compounds that are, or are up to 15, 16, 17, 18, 19, 20, 21, 22, 23, 24, or 25 nucleobases in length comprise no more than 6, no more than 5, no more than 4, no more than 3, no more than 2, or no more than 1 non-complementary nucleobase(s) relative to a target nucleic acid, such as a RUNX1 nucleic acid, or specified portion thereof.

In certain embodiments, antisense compounds that are, or are up to 18, 19, 20, 21, or 22 nucleobases in length comprise no more than 4, no more than 3, no more than 2, or no more than 1 non-complementary nucleobase(s) relative to a target nucleic acid, such as a RUNX1 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds, are complementary to at least an 11 nucleobase portion of a target segment. In certain embodiments, the antisense compounds, are complementary to at least a 12 nucleobase portion of a target segment. In certain embodiments, the antisense compounds, are complementary to at least a 13 nucleobase portion of a target segment. In certain embodiments, the antisense compounds, are complementary to at least a 14 nucleobase portion of a target segment. In certain embodiments, the antisense compounds, are complementary to at least a 15 nucleobase portion of a target segment. Also contemplated are antisense compounds that are complementary to at least a 9, 10, 11, 12, 13, 14, 15, 16, 17, 18, 19, 20, 21, 22, 23, 24, 25, 26, 27, 28, 29, 30 or more nucleobase portion of a target segment, or a range defined by any two of these values.

### Identity

The antisense compounds provided herein may also have a defined percent identity to a particular nucleotide sequence, SEQ ID NO, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds are at least 70%, 75%, 80%, 85%, 90%, 95%, 96%, 97%, 98%, 99% or 100% identical to one or more of the SEQ ID NO: 20-SEQ ID NO: 38, or a portion thereof, disclosed herein.

In certain embodiments, a portion of the antisense compound is compared to an equal length portion of the target nucleic acid. In certain embodiments, a 10, 11, 12, 13, 14, 15, 16, 17, 18 19, 20, 21, 22, 23, 24, 25, 26, 27, 28, 29, or 30 nucleobase portion is compared to an equal length portion of the target nucleic acid.

In certain embodiments, a portion of the antisense oligonucleotide is compared to an equal length portion of the target nucleic acid. In certain embodiments, a 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 18, 19, 20, 21, or 22 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a RUNX1 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage. In certain embodiments, the antisense compounds targeted to a RUNX1 nucleic acid comprise at least one phosphodiester linkage and at least one phosphorothioate linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R²) (R, R₁ and R₂ are each independently H, C₁-C₁₂ alkyl or a protecting group) and combinations thereof.

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O-CH₂—C(═O)—N(Rₘ)(Rₙ), and O—CH₂—C(═O)—N(R₁)—(CH₂)₂—N(Rₘ)(Rₙ), where each R₁, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—₂ S′; 4′-(CH₂)₂—O-2′ (ENA); 4′-CH(CH₃)—O-2′ and 4′-CH(CH₂OCH₃)—O-2′.

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243.

### Compositions and Methods for Formulating Pharmaceutical Compositions

In some embodiments, an oligomeric compound described herein, or a salt thereof or an oligomeric duplex described herein comprises an antisense compound. In some embodiments, a pharmaceutical composition comprises an oligomeric compound, or a salt thereof, or an oligomeric duplex and a pharmaceutically acceptable carrier or diluent.

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a RUNX1 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a RUNX1 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps.

Oligomeric compounds may be chemically linked to one or more moieties or conjugates which enhance the oligomeric compound properties such as activity, cellular distribution or cellular uptake. These moieties or conjugates can include conjugate groups covalently bound to functional groups such as primary or secondary hydroxyl groups. Conjugate groups of the invention include inter-calators, reporter molecules, polyamines, polyamides, polyethylene glycols, polyethers, groups that enhance the pharmacodynamic properties of oligomers, and groups that enhance the pharmacokinetic properties of oligomers. Additional conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention.

Oligomeric compounds can also be modified to have one or more stabilizing groups that are generally attached to one or both termini of an oligomeric compound to enhance properties such as for example nuclease stability. Included in stabilizing groups are cap structures. By “cap structure or terminal cap moiety” is meant chemical modifications, which have been incorporated at either terminus of oligonucleotides. These terminal modifications protect the oligomeric compounds having terminal nucleic acid molecules from exonuclease degradation, and can improve delivery and/or localization within a cell. The cap can be present at either the 5′-terminus (5′-cap) or at the 3′-terminus (3′-cap) or can be present on both termini of a single strand, or one or more termini of both strands of a double-stranded compound. This cap structure is not to be confused with the inverted methylguanosine “5′ cap” present at the 5′ end of native mRNA molecules.

In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. For siRNA constructs, the 5′ end (5′ cap) is commonly but not limited to 5′-hydroxyl or 5′-phosphate.

Particularly suitable 3′-cap structures include, for example 4′,5′-methylene nucleotide; I-(beta-D-erythrofuranosyl) nucleotide; 4′-thio nucleotide, carbocyclic nucleotide; 5′-amino-alkvl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

### Methods of Treatment

In some embodiments, the oligomeric compounds, or a salt thereof, the oligomeric duplexes, or the pharmaceutical compositions described herein are used in a method of improving cognitive function in a subject in need thereof, comprising administering the oligomeric compounds, or a salt thereof, the oligomeric duplexes, or the pharmaceutical compositions to the subject. In some embodiments the subject has an overexpression of RUNX1. In some embodiments, the subject has Down syndrome. In some embodiments, the oligomeric compound, or a salt thereof, the oligomeric duplex, or the pharmaceutical composition is used in a method for treating a disease associated with RUNX1 in a subject in need thereof comprising administering the oligomeric compound, or a salt thereof, the oligomeric duplex, or the pharmaceutical composition to the subject.

Provided herein are methods for treating an individual with Down syndrome (DS). Treatment encompasses decreasing overexpression of RUNX1 and in turn modifying transcriptomic, morphological, and functional aspects of microglia. Treatment further encompasses normalizing microglia in DS brains, and in turn improving memory and potentially additional cognitive attributes for individuals with DS. In some embodiments, such treatment methods comprise the administration to the individual with DS a therapeutically effective amount of a pharmaceutical composition comprising an antisense compound or oligonucleotide targeted to RUNX1.

In one aspect, provided herein are methods for treating cognitive decline in an individual associated with a neurogenerative diseases or disorder comprising administering an effective amount of an antisense compound or oligonucleotide targeted to RUNX1 provided herein. In certain embodiments, the neurodegenerative disease or disorder is any neurodegenerative disease or disorder characterized by cognitive decline or cognitive loss. In specific embodiments, the neurodegenerative disease or disorder is Alzheimer's disease, Parkinson's disease, Down syndrome, Down syndrome associated dementia, dementia, prion disease, Amyotrophic lateral sclerosis, Naso Hakola disorder, spinal muscular atrophy, or spinocerebellar ataxia. In certain embodiments, the neurodegenerative disease or disorder is Alzheimer's disease. In some embodiments, the neurodegenerative disease or disorder is Down syndrome. In certain embodiments, the neurodegenerative disease or disorder is Down syndrome associated dementia. In certain embodiments, antisense compounds or oligonucleotides targeted to RUNX1 (e.g., as provided herein) decrease inflammation and slow cognitive decline in an individual with a neurodegenerative disease or disorder (e.g., a neurodegenerative disease or disorder provided herein).

One embodiment provides a method of treating a disease associated with runt-related transcription factor 1 (RUNX1) in a subject in need thereof, comprising administering an oligomeric compound, or a salt thereof, to the subject, said oligomeric compound comprising a modified oligonucleotide consisting of 17-23 linked nucleosides, wherein the modified oligonucleotide has a nucleobase sequence that is at least 80% complementary to any contiguous 17-23 nucleobase sequences of RUNX1 mRNA or RUNX1 gene, when measured across the entire nucleobase sequence of the modified oligonucleotide, wherein the modified oligonucleotide comprises at least one modification selected from a modified sugar, a sugar surrogate, and a modified internucleoside linkage, and wherein the RUNX1 gene comprises a sense strand and an antisense strand. In some embodiments, the oligomeric compound reduces RUNX1 mRNA levels measured by quantitative real-time PCR in human immortalized microglia-like cells in comparison to mRNA levels measured by quantitative real-time PCR in human immortalized microglia-like cells that were not treated with oligomeric compound. In some embodiments, the modified oligonucleotide has a nucleobase sequence that is at least 90% or 95% complementary to any contiguous 17-23 nucleobase sequences of RUNX1 mRNA or RUNX1 gene, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 gene sense strand or RUNX1 gene antisense strand, when measured across the entire nucleobase sequence of the modified oligonucleotide. In some embodiments, the modified oligonucleotide has a nucleobase sequence that is at least 90% or 95% complementary to any contiguous 17-23 nucleobase sequences of a portion of RUNX1 gene sense strand or RUNX1 gene antisense strand corresponding to the sequence of RUNX1 mRNA, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 mRNA, when measured across the entire nucleobase sequence of the modified oligonucleotide.

One embodiment provides a method of treating a disease associated with runt-related transcription factor 1 (RUNX1) in a subject in need thereof, comprising administering an inhibitor of runt-related transcription factor 1 (RLUNX1) synthesis. In some embodiments, the inhibitor of RUNX1 synthesis is an oligomeric compound, or a salt thereof, said oligomeric compound comprising a modified oligonucleotide consisting of 17-23 linked nucleosides, wherein the modified oligonucleotide has a nucleobase sequence that is at least 80% complementary to any contiguous 17-23 nucleobase sequences of RUNX1 mRNA or RUNX1 gene, when measured across the entire nucleobase sequence of the modified oligonucleotide, wherein the modified oligonucleotide comprises at least one modification selected from a modified sugar, a sugar surrogate, and a modified internucleoside linkage, and wherein the RUNX1 gene comprises a sense strand and antisense strand. In some embodiments, the modified oligonucleotide has a nucleobase sequence that is at least 90% or 95% complementary to any contiguous 17-23 nucleobase sequences of RUNX1 mRNA or RUNX1 gene, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX gene sense strand or RUNX1 gene antisense strand, when measured across the entire nucleobase sequence of the modified oligonucleotide. In some embodiments, the modified oligonucleotide has a nucleobase sequence that is at least 90% or 95% complementary to any contiguous 17-23 nucleobase sequences of a portion of RUNX1 gene sense strand or RUNX1 gene antisense strand corresponding to the sequence of RUNX1 mRNA, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 mRNA, when measured across the entire nucleobase sequence of the modified oligonucleotide. In some embodiments, the inhibitor of RUNX1 synthesis reduces expression of RUNX1 by about 1.5 folds to about 50 folds. In some embodiments, the modified oligonucleotide comprises at least one modified internucleoside linkage, and the modified internucleoside linkage is a phosphorothioate linkage, the modified oligonucleotide comprises at least 10-17 phosphorothioate linkages. In some embodiments, the oligomeric compound comprises a modified oligonucleotide consisting of 20 linked nucleosides comprising 19 phosphorothioate linkages. In some embodiments, the subject has an overexpression of RUNX1. In some embodiments, the subject has Down syndrome. In some embodiments, the subject has Alzheimer's disease.

One embodiment provides a method of improving cognitive function in a subject having an overexpression of RUNX1 in a cell, comprising administering an inhibitor of runt-related transcription factor 1 (RUNX1) synthesis to the subject. In some embodiments, the inhibitor of RUNX1 synthesis is an antisense compound. In some embodiments, the antisense compound is an antisense oligonucleotide, siRNA, shRNA, or ssRNA. In some embodiments, the antisense oligonucleotide is a modified oligonucleotide consisting of 17-23 linked nucleosides, wherein the modified oligonucleotide has a nucleobase sequence that is at least 80% complementary to any contiguous 17-23 nucleobase sequences of RUNX1 mRNA or RUNX1 gene, when measured across the entire nucleobase sequence of the modified oligonucleotide, wherein the modified oligonucleotide comprises at least one modification selected from a modified sugar, a sugar surrogate, and a modified internucleoside linkage, and wherein the RUNX1 gene comprises a sense strand and an antisense strand. In some embodiments, the modified oligonucleotide has a nucleobase sequence that is at least 90% or 95% complementary to any contiguous 17-23 nucleobase sequences of RUNX1 mRNA or RUNX1 gene, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 gene sense strand or RUNX1 gene antisense strand, when measured across the entire nucleobase sequence of the modified oligonucleotide. In some embodiments, the modified oligonucleotide has a nucleobase sequence that is at least 90% or 95% complementary to any contiguous 17-23 nucleobase sequences of a portion of RUNX1 gene sense strand or RUNX1 gene antisense strand corresponding to the sequence of RUNX1 mRNA, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 mRNA, when measured across the entire nucleobase sequence of the modified oligonucleotide. In some embodiments, the inhibitor of RUNX1 synthesis reduces expression of RUNX1 by about 1.5 folds to about 50 folds. In some embodiments, the modified oligonucleotide comprises at least one modified internucleoside linkage, and the modified internucleoside linkage is a phosphorothioate linkage. In some embodiments, the modified oligonucleotide comprises at least 10-17 phosphorothioate linkages. In some embodiments, the oligomeric compound comprises a modified oligonucleotide consisting of 20 linked nucleosides comprising 19 phosphorothioate linkages. In some embodiments, the subject has an overexpression of RUNX1. In some embodiments, the subject has Down syndrome. In some embodiments, the subject has Alzheimer's disease.

One embodiment provides a method of improving cognitive function in a subject having an overexpression of RUNX1 in a cell, comprising administering an inhibitor of runt-related transcription factor 1 (RUNX1) synthesis to the subject. In some embodiments, the inhibitor of RUNX1 synthesis is an antisense compound. In some embodiments, the antisense compound is an antisense oligonucleotide, siRNA, shRNA, or ssRNA. In some embodiments, the antisense oligonucleotide is a modified oligonucleotide consisting of 17-23 linked nucleosides, wherein the modified oligonucleotide has a nucleobase sequence that is at least 80% complementary to any contiguous 17-23 nucleobase sequences of RUNX1 mRNA or RUNX1 gene, when measured across the entire nucleobase sequence of the modified oligonucleotide, wherein the modified oligonucleotide comprises at least one modification selected from a modified sugar, a sugar surrogate, and a modified internucleoside linkage, and wherein the RUNX1 gene comprises a sense strand and an antisense strand. In some embodiments, the modified oligonucleotide has a nucleobase sequence that is at least 90% or 95% complementary to any contiguous 17-23 nucleobase sequences of RUNX1 mRNA or RUNX1 gene, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 gene sense strand or RUNX1 gene antisense strand, when measured across the entire nucleobase sequence of the modified oligonucleotide. In some embodiments, the modified oligonucleotide has a nucleobase sequence that is at least 90% or 95% complementary to any contiguous 17-23 nucleobase sequences of a portion of RUNX1 gene sense strand or RUNX1 gene antisense strand corresponding to the sequence of RUNX1 mRNA, when measured across the entire nucleobase sequence of the modified oligonucleotide. In some embodiments, the modified oligonucleotide has a nucleobase sequence that is at least 90% or 95% complementary to any contiguous 17-23 nucleobase sequences of RUNX1 mRNA, when measured across the entire nucleobase sequence of the modified oligonucleotide. In some embodiments, the inhibitor of RUNX1 synthesis reduces expression of RUNX1 by about 1.5 folds to about 50 folds. In some embodiments, the modified oligonucleotide comprises at least one modified internucleoside linkage, and the modified internucleoside linkage is a phosphorothioate linkage. In some embodiments, the modified oligonucleotide comprises at least 10-17 phosphorothioate linkages. In some embodiments, the oligomeric compound comprises a modified oligonucleotide consisting of 20 linked nucleosides comprising 19 phosphorothioate linkages. In some embodiments, wherein the antisense oligonucleotide comprises an oligomeric compound, or a salt thereof, described herein. In some embodiments, the subject has Down syndrome. In some embodiments, the subject has Alzheimer's disease.

One embodiment provides a method of improving cognitive function in a Down syndrome subject comprising identifying a Down syndrome subject in need of treatment for cognitive disability and administering to the subject a therapeutically effective amount of a RUNX1 inhibitor thereby reducing the expression of RUNX1 in the subject's microglia and increasing cognitive function in the subject.

One embodiment provides a method of treating cognitive loss associated with a neurodegenerative disease or disorder, the method comprising: identifying a subject in need of treatment for cognitive decline; and administering a therapeutically effective amount of a RUNX1 inhibitor to the subject thereby reducing the expression of RUNX1 in microglia of the subject and treating the cognitive loss in the subject. In some embodiments, the subject is diagnosed with a neurodegenerative disease or disorder. In certain embodiments, the neurodegenerative disease or disorder comprises Alzheimer's disease, Parkinson's disease, Down syndrome, Down syndrome associated dementia, prion disease, Amyotrophic lateral sclerosis, Nasu Hakola disorder, spinal muscular atrophy, or spinocerebellar ataxia. In some embodiments, the neurodegenerative disease or disorder is Down syndrome or Down syndrome associated dementia. In certain embodiments, the neurodegenerative disease or disorder is Alzheimer's disease.

In some embodiments, inhibitors of RUNX1 synthesis described herein reduce expression of RUNX1 by about 1.5 folds to about 50 folds. In some embodiments, inhibitors of RUNX1 described herein synthesis can reduce expression of RUNX1 by about 1.5 folds to about 2 folds, about 1.5 folds to about 3 folds, about 1.5 folds to about 4 folds, about 1.5 folds to about 5 folds, about 1.5 folds to about 10 folds, about 1.5 folds to about 15 folds, about 1.5 folds to about 20 folds, about 1.5 folds to about 25 folds, about 1.5 folds to about 30 folds, about 1.5 folds to about 40 folds, about 1.5 folds to about 50 folds, about 2 folds to about 3 folds, about 2 folds to about 4 folds, about 2 folds to about 5 folds, about 2 folds to about 10 folds, about 2 folds to about 15 folds, about 2 folds to about 20 folds, about 2 folds to about 25 folds, about 2 folds to about 30 folds, about 2 folds to about 40 folds, about 2 folds to about 50 folds, about 3 folds to about 4 folds, about 3 folds to about 5 folds, about 3 folds to about 10 folds, about 3 folds to about 15 folds, about 3 folds to about 20 folds, about 3 folds to about 25 folds, about 3 folds to about 30 folds, about 3 folds to about 40 folds, about 3 folds to about 50 folds, about 4 folds to about 5 folds, about 4 folds to about 10 folds, about 4 folds to about 15 folds, about 4 folds to about 20 folds, about 4 folds to about 25 folds, about 4 folds to about 30 folds, about 4 folds to about 40 folds, about 4 folds to about 50 folds, about 5 folds to about 10 folds, about 5 folds to about 15 folds, about 5 folds to about 20 folds, about 5 folds to about 25 folds, about 5 folds to about 30 folds, about 5 folds to about 40 folds, about 5 folds to about 50 folds, about 10 folds to about 15 folds, about 10 folds to about 20 folds, about 10 folds to about 25 folds, about 10 folds to about 30 folds, about 10 folds to about 40 folds, about 10 folds to about 50 folds, about 15 folds to about 20 folds, about 15 folds to about 25 folds, about 15 folds to about 30 folds, about 15 folds to about 40 folds, about 15 folds to about 50 folds, about 20 folds to about 25 folds, about 20 folds to about 30 folds, about 20 folds to about 40 folds, about 20 folds to about 50 folds, about 25 folds to about 30 folds, about 25 folds to about 40 folds, about 25 folds to about 50 folds, about 30 folds to about 40 folds, about 30 folds to about 50 folds, or about 40 folds to about 50 folds. In some embodiments, inhibitors of RUNX1 synthesis described herein can reduce expression of RUNX1 by about 1.5 folds, about 2 folds, about 3 folds, about 4 folds, about 5 folds, about 10 folds, about 15 folds, about 20 folds, about 25 folds, about 30 folds, about 40 folds, or about 50 folds. In some embodiments, inhibitors of RUNX1 synthesis described herein can reduce expression of RUNX1 by at least about 1.5 folds, about 2 folds, about 3 folds, about 4 folds, about 5 folds, about 10 folds, about 15 folds, about 20 folds, about 25 folds, about 30 folds, or about 40 folds.

The present invention employs antisense compounds, particularly antisense oligonucleotides, for use in modulating the function of nucleic acid molecules encoding RUNX1, ultimately modulating the amount of RUNX1 protein produced. A suitable form of modulation is inhibition of nucleic acid molecules encoding RUNX1, which is evidenced by a reduction in the levels of nucleic acids encoding RUNX1. Accordingly, disclosed herein are antisense compounds, including antisense oligonucleotides, for use in inhibiting the expression of nucleic acid molecules encoding RUNX1, i.e., reducing the levels of nucleic acid molecules encoding RUNX1. As used herein, the terms “target nucleic acid” and “nucleic acid molecule encoding RUNX1” have been used for convenience to encompass DNA encoding RUNX1, RNA (including pre-mRNA and mRNA or portions thereof) transcribed from such DNA, and also cDNA derived from such RNA. Antisense oligonucleotides which hybridize to and modulate the expression of one or more nucleic acids encoding RUNX1 are considered to be “targeted to RUNX1”.

### Modified and Alternate Nucleobases

Oligomeric compounds can also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). A “substitution” is the replacement of an unmodified or natural base with another unmodified or natural base. “Modified” nucleobases mean other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), pyridoindole cytidine (H-pyrido(3′,2′4,5)pyrrolo(2,3-d)pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Certain nucleobase modifications increase the binding affinity of the compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications. It is understood in the art that modification of the base does not entail such chemical modifications as to produce substitutions in a nucleic acid sequence.

Oligomeric compounds of the present invention can also include polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs.

### Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

### Oligonucleotide Synthesis

Oligomeric compounds and phosphoramidites are made by methods well known to those skilled in the art. Oligomerization of modified and unmodified nucleosides is performed according to literature procedures for DNA like compounds (Protocols for Oligonucleotides and Analogs, Ed. Agrawal (1993), Humana Press) and/or RNA like compounds (Scaringe, Methods (2001), 23 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. Alternatively, oligomers may be purchased from various oligonucleotide synthesis companies such as, for example, Dharmacon Research Inc., (Lafayette, Colo.).

Irrespective of the particular protocol used, the oligomeric compounds used in accordance with this invention may be conveniently and routinely made through the well-known technique of solid phase synthesis. Equipment for such synthesis is sold by several vendors including, for example, Applied Biosystems (Foster City, Calif). Any other means for such synthesis known in the art may additionally or alternatively be employed (including solution phase synthesis).

Methods of isolation and analysis of oligonucleotides are well known in the art. A 96-well plate format is particularly useful for the synthesis, isolation and analysis of oligonucleotides for small scale applications.

### Modulation of Target Expression

Modulation of expression of a target nucleic acid can be achieved through alteration of any number of nucleic acid (DNA or RNA) functions. “Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. The functions of DNA to be modulated can include replication and transcription. Replication and transcription, for example, can be from an endogenous cellular template, a vector, a plasmid construct or otherwise. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of RUNX1. Thus, in one embodiment modulation of expression can mean increase or decrease in target RNA or protein levels. In another embodiment modulation of expression can mean an increase or decrease of one or more RNA splice products, or a change in the ratio of two or more splice products.

### EXAMPLES

### I. Chemical Synthesis

### Example 1: Antisense Oligonucleotide Synthesis

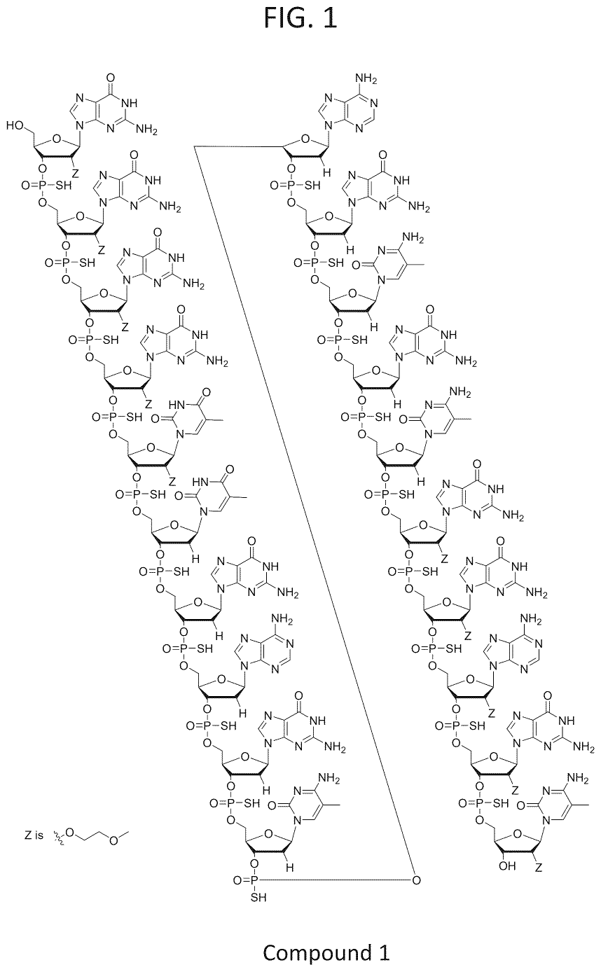

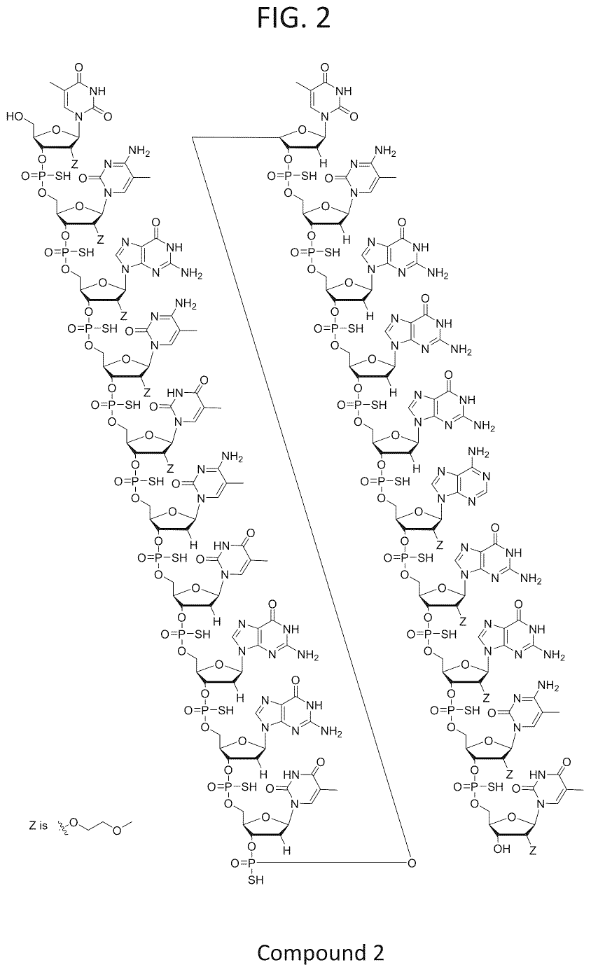

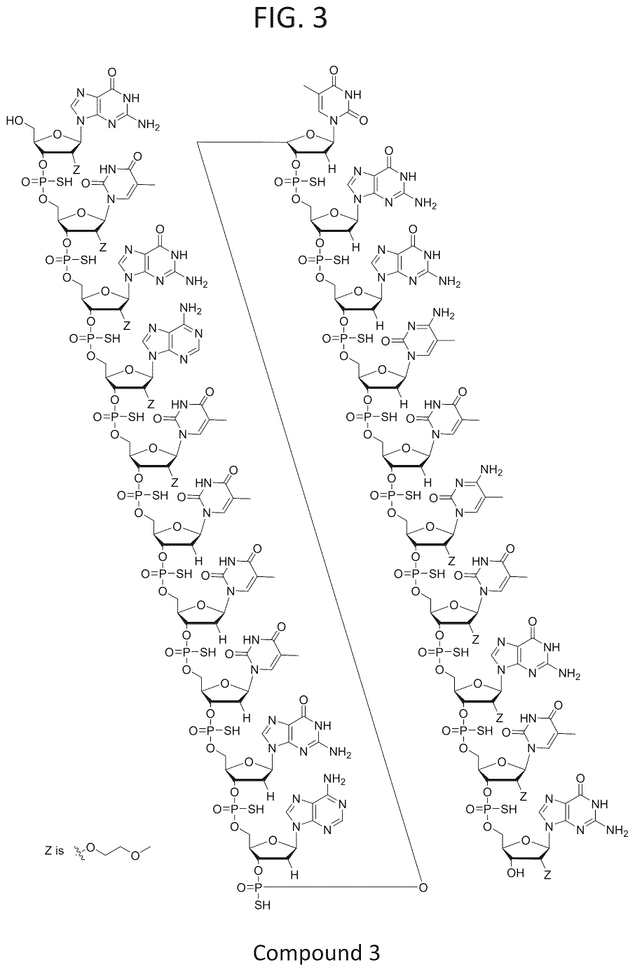

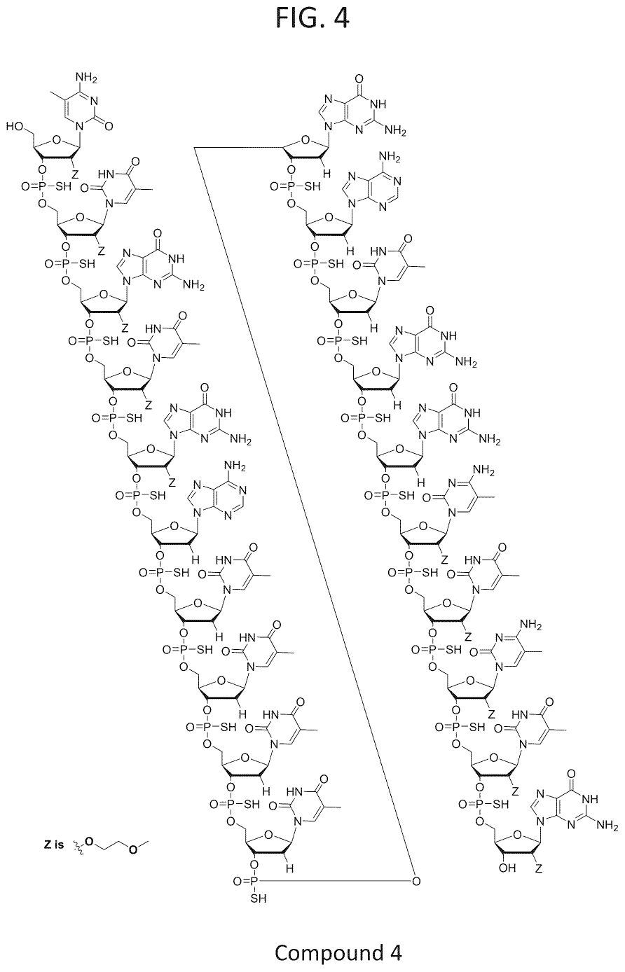

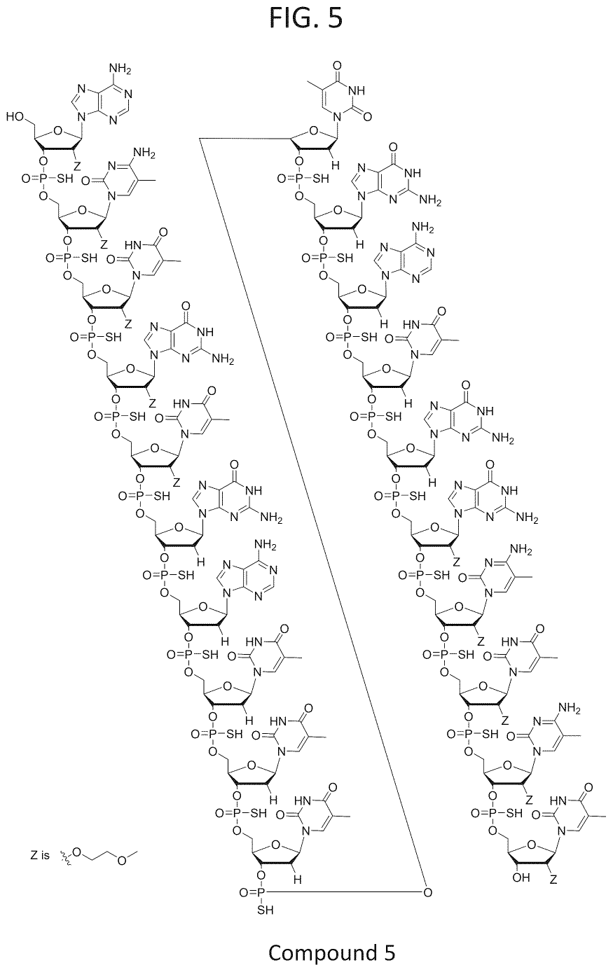

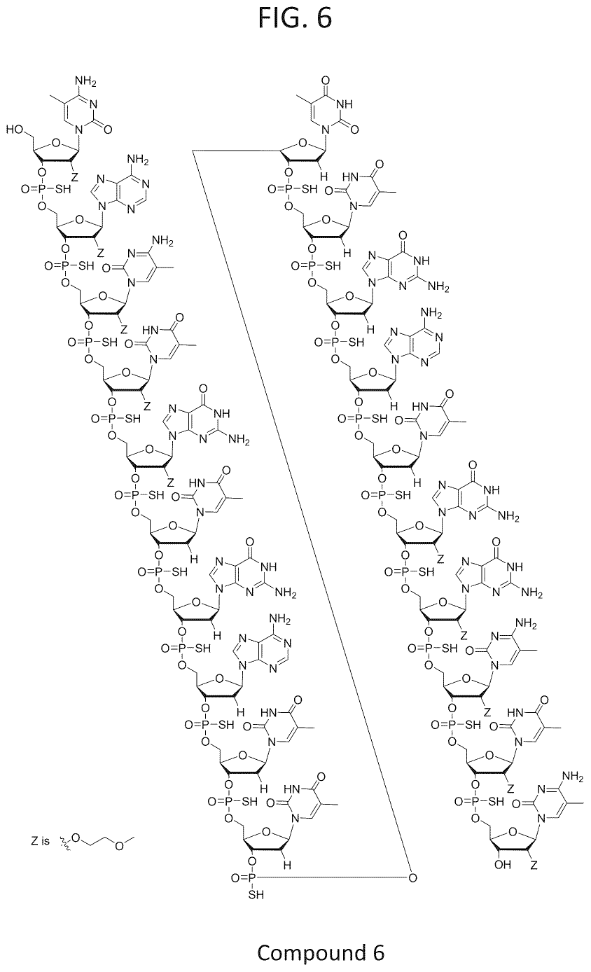

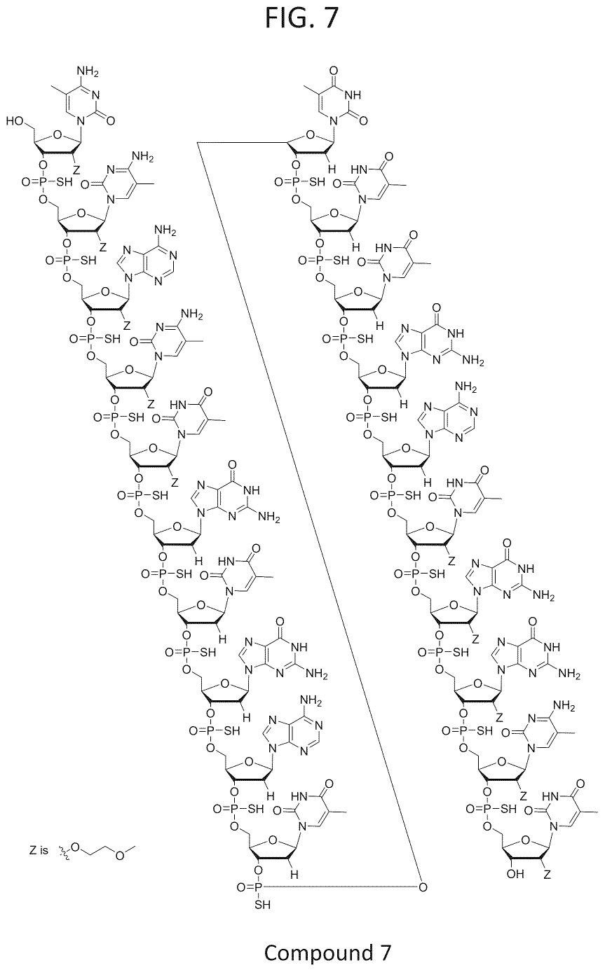

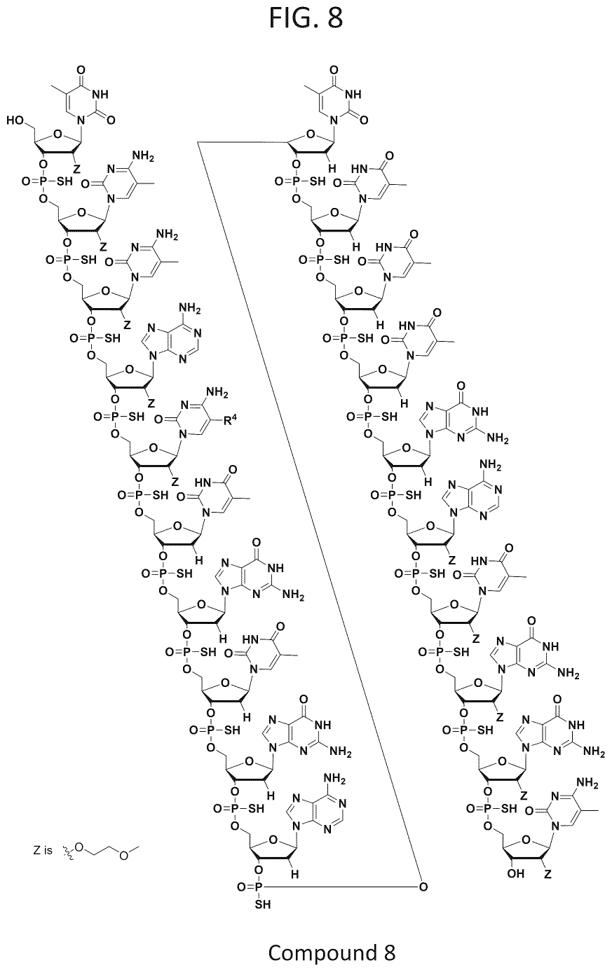

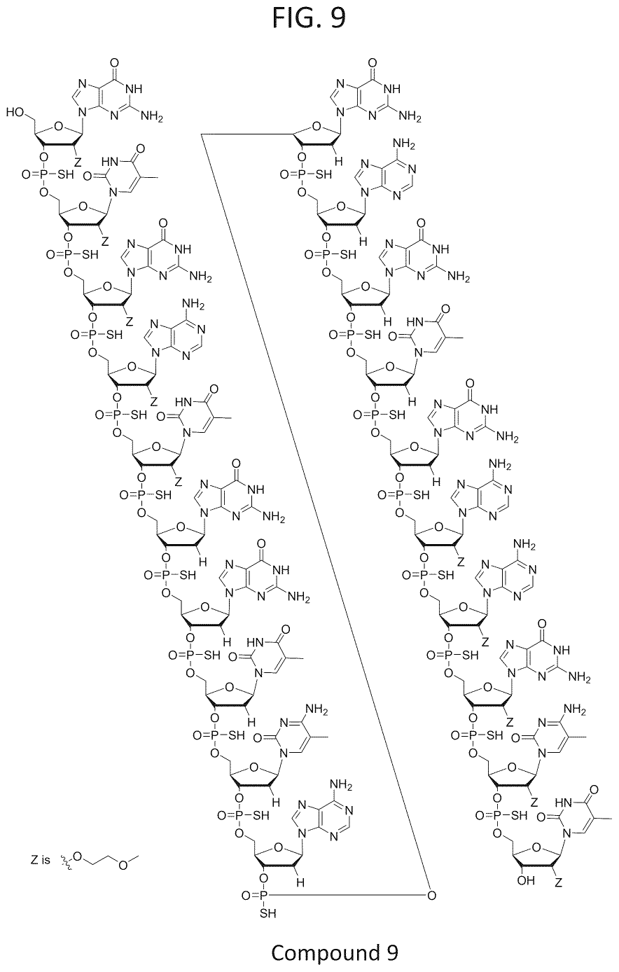

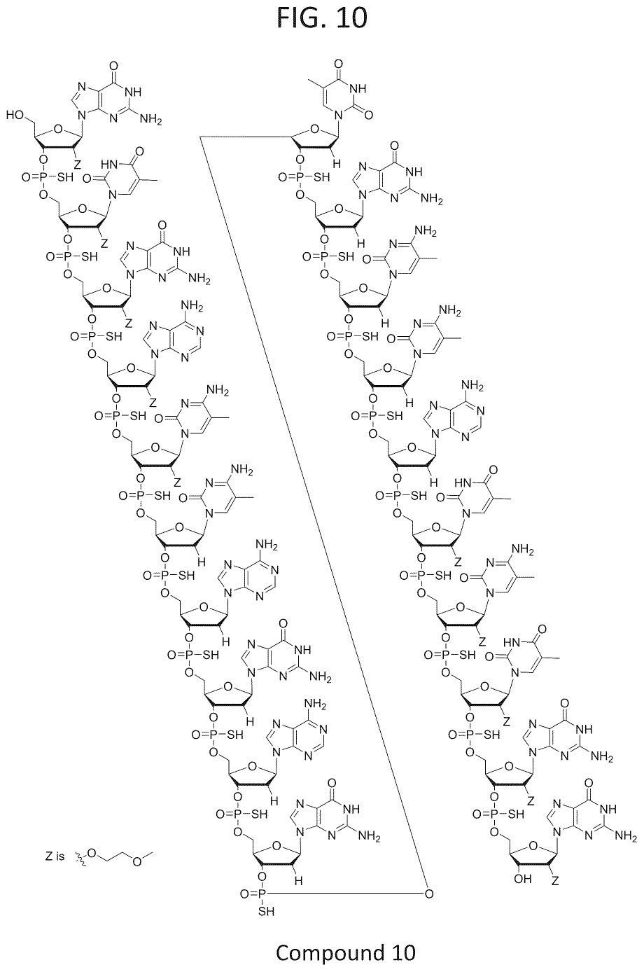

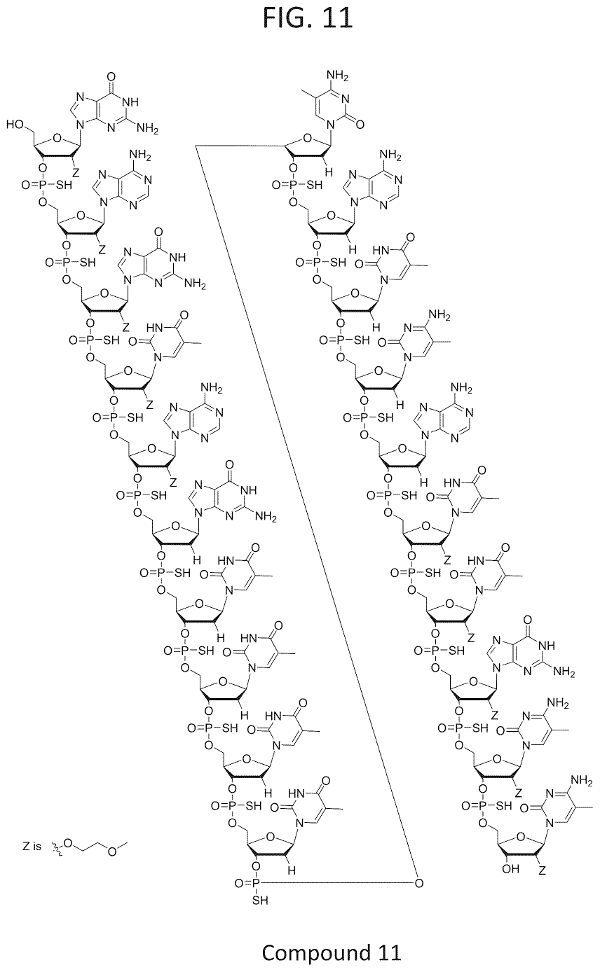

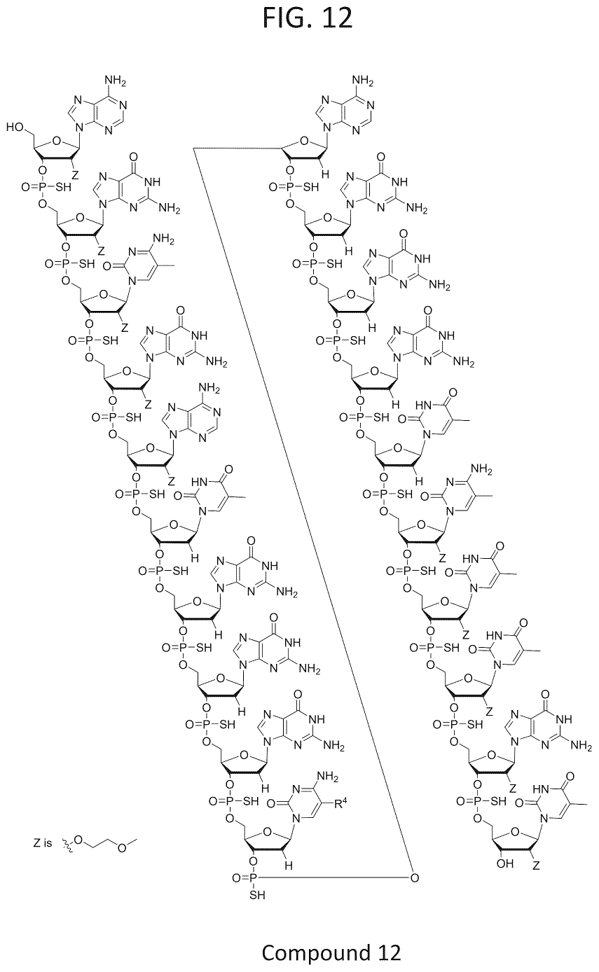

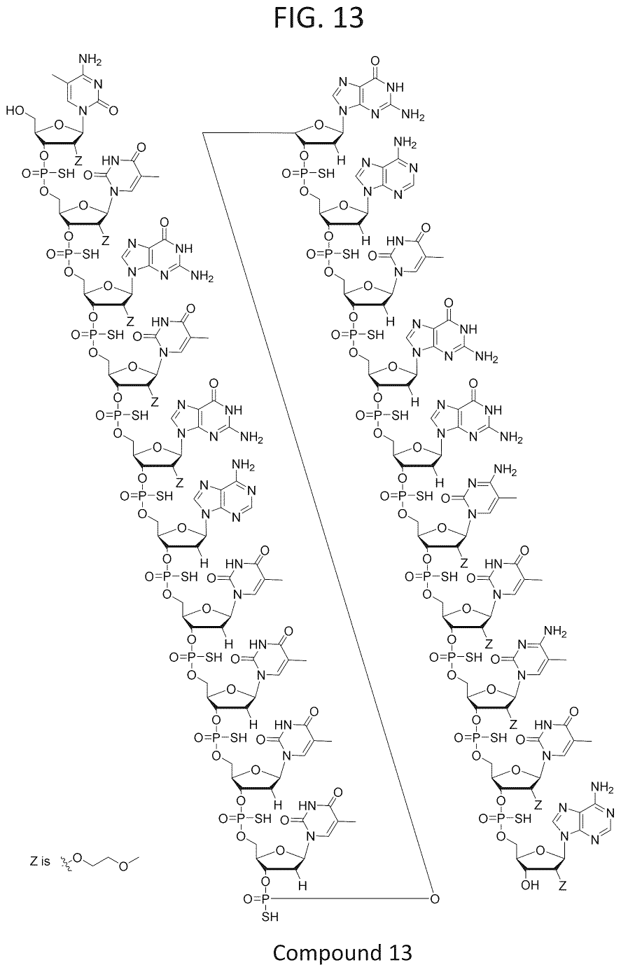

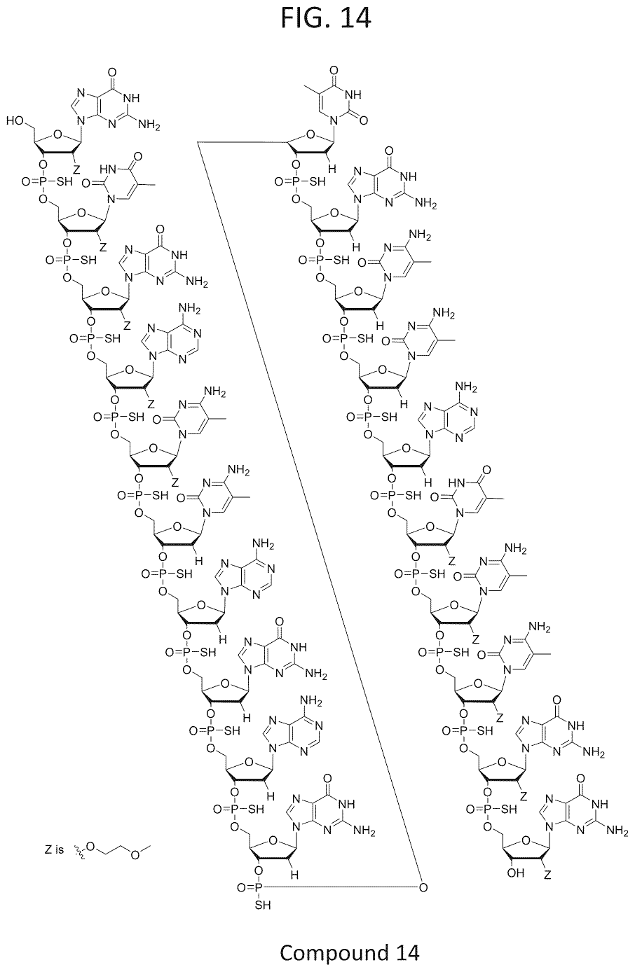

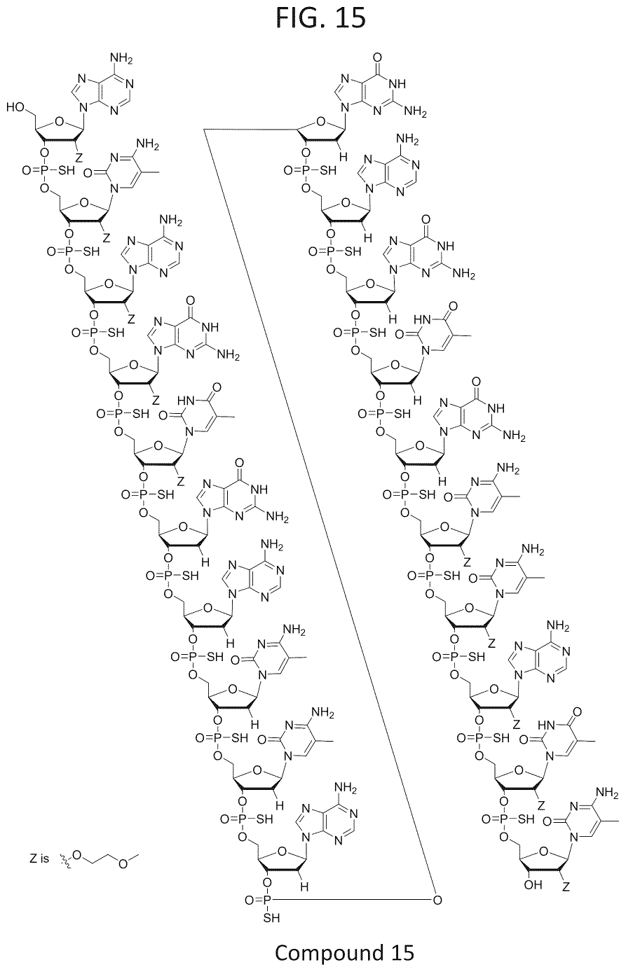

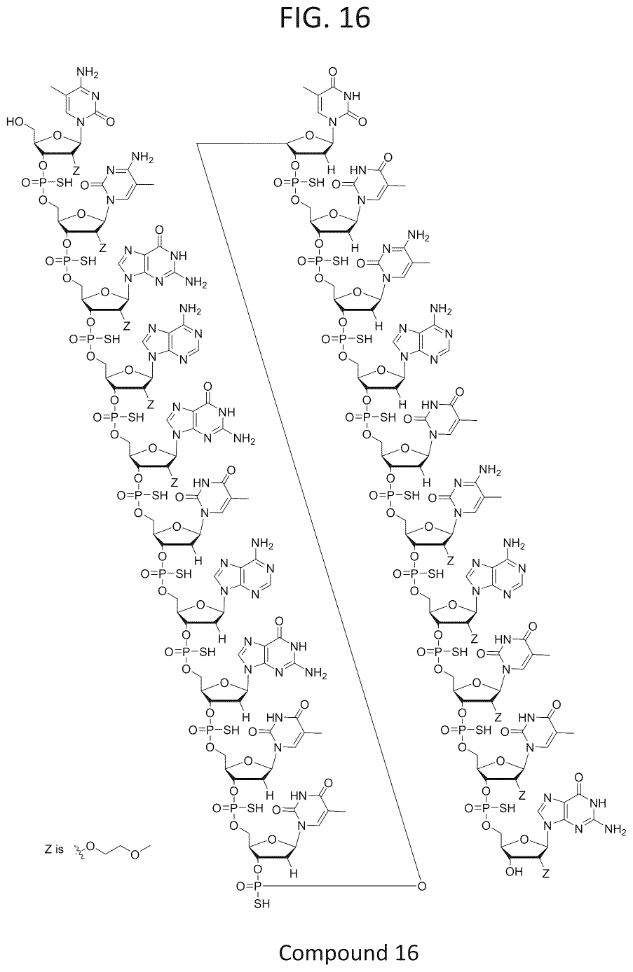

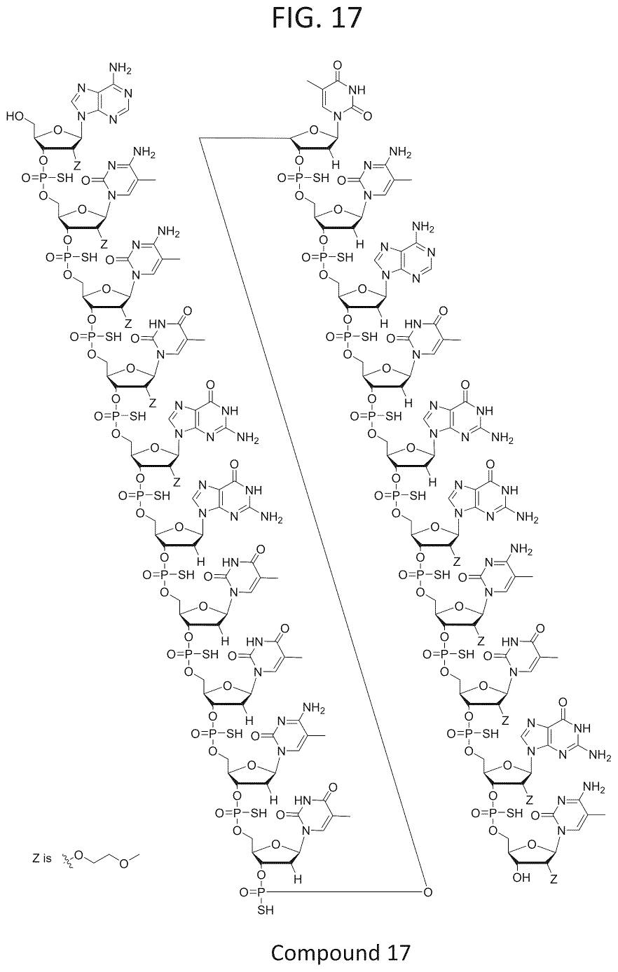

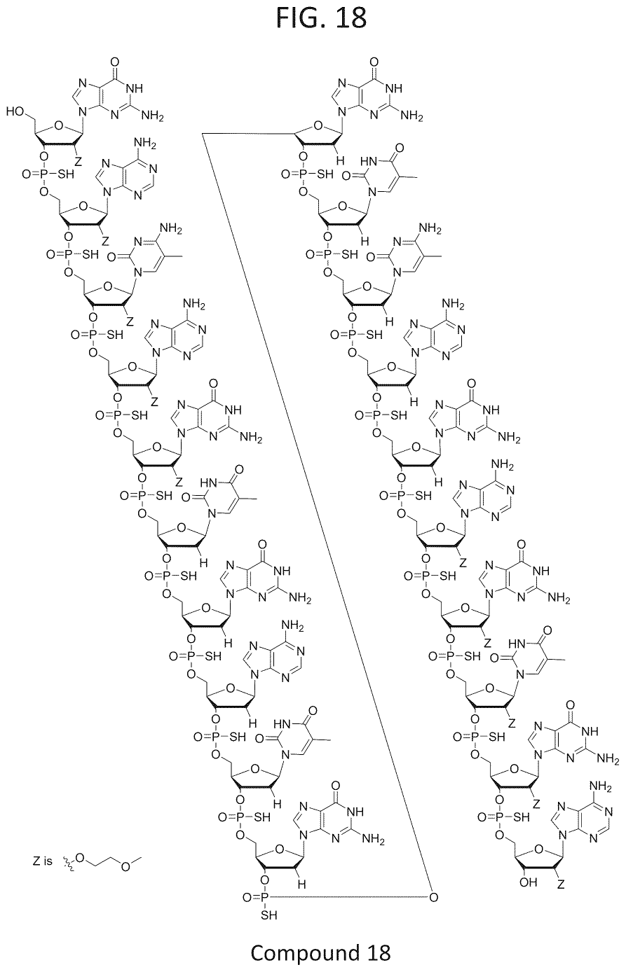

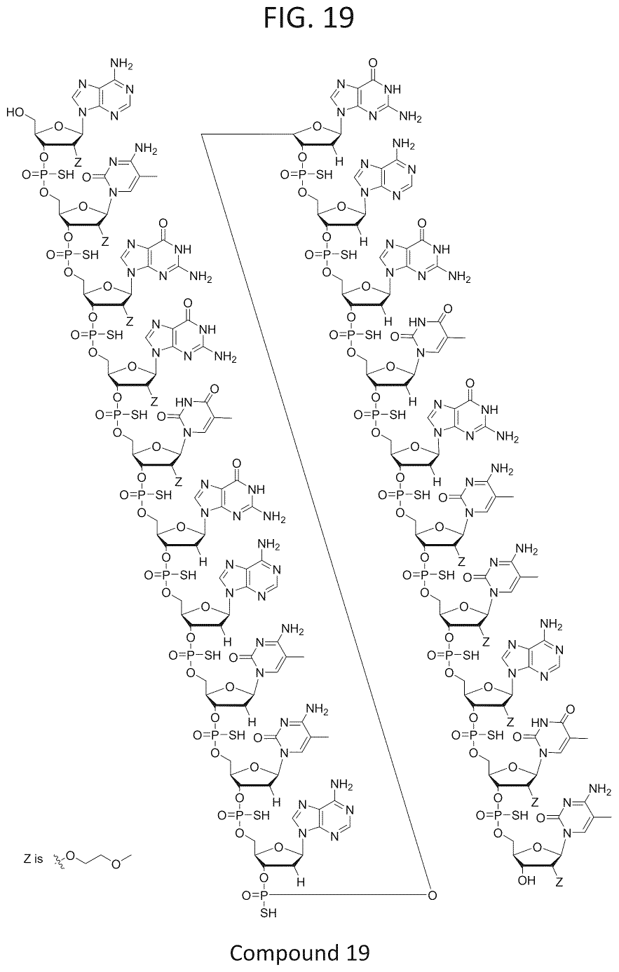

ASOs covering 19 sequences were synthesized using a gapmer design. In each of the synthesized ASOs, each cytosine is a 5-methyl cytosine. In brief, ASOs were 20 nucleotides in length with the center 10 nucleotides being standard, unmodified, 2′-deoxynucleotides. The flanking 5 nucleotides on either side had 2′-O-methoxyethyl (2′ MOE) modifications to the sugars. All nucleotides were connected via phosphorothioate bonds. Designed ASOs target a wide array of exons on RUNX1. All ASOs were synthesized at Integrated DNA Technologies (IDT). Example ASOs are provided in FIG. 1-FIG. 19.

### II. Biological Evaluations

### Example 1: Effect of MOE Gapmers with Internucleoside Linkages on Mouse Runx1 in Mouse Microglia by Free Uptake, Single Dose

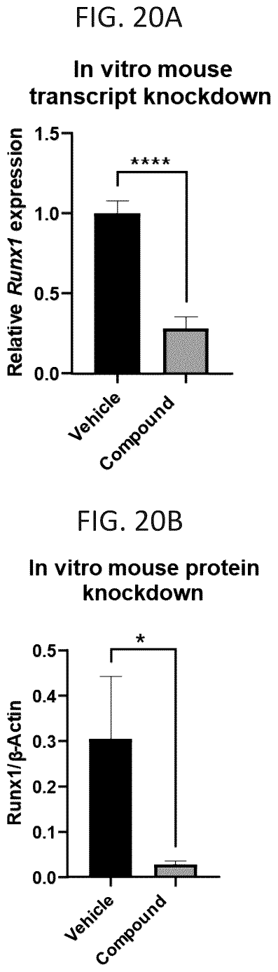

Summary: Microglia were extracted from the cortices of 8-day old mice and were cultured in vitro for 3 days. After 3 days, ASOs were added to the media to obtain a final concentration of 10 M in the media. ASOs were left on the cells for 7 days. Media was removed, cells were lysed, and RNA and protein were extracted. Levels of RNA were determined using RT-qPCR with probes against Runx1 and control probes against β-Actin. Protein knockdown was confirmed via Western blot with antibodies targeting Runx1. Effect of an exemplary ASO compound on mouse transcript knockdown and mouse protein knockdown in vitro is provided in FIG. 20A and FIG. 20B.

Modified oligonucleotides complementary to both a human and mouse Runx1 nucleic acid were designed and tested for their effect on Runx1 transcript in vitro. The modified oligonucleotides were tested in a series of experiments that had similar culture conditions. Mouse primary microglia were plated at a concentration of 40,000 cells per well and were allowed to mature for 3 days in vitro. Modified oligonucleotides were added to culture medium to achieve a final concentration of 10,000 nM as well as no oligonucleotide diluent controls. After approximately 7 days, RNA was isolated from the cells and Runx1 mRNA levels were measured by quantitative real-time PCR. Mouse Tagman Assay ID Mm01213404 was used to measure mRNA levels. Runx1 levels were compared to the housekeeping gene Beta Actin Mouse Taqman Assay ID Mm02619580_g1. Results are presented in Table 1 as relative expression of Runx1, normalized to the no oligonucleotide control. A value of 0% reduction indicates that the compound had no effect or increased RNA concentrations in the cell. The modified oligonucleotides in Table 1 are 5-10-5 mixed MOE gapmers. The gapmers are 20 nucleotides in length, wherein the central gap segment comprises ten 2′-deoxynucleosides and is flanked on either side by segments of five 2′MOE nucleosides. The internucleoside linkages are phosphorothioate linkages.

Each modified oligonucleotide listed in Table 1 below is complementary to human RUNX1 nucleic acid sequences SEQ ID NO: 1-SEQ ID NO: 19, as indicated. For each of the ASOs listed in Table 1, each cytosine is a 5-methyl cytosine. A value of 0% reduction indicates that the compound had no effect or increased mRNA concentrations in the cell. As shown below, modified oligonucleotides complementary to mouse Runx1 reduced the amount of mouse Runx1 mRNA.

### Example 2: Effect of Modified Oligonucleotides on Mouse Runx1 in Mouse Microglia by Free Uptake, Multiple Doses

Modified oligonucleotides selected from Table 1 were tested at various doses in mouse primary microglia. Mouse primary microglia were plated at a concentration of 40,000 cells per well in a 96 well plate and were allowed to mature for 3 days in vitro. After three days culture, modified oligonucleotides were added to culture medium to achieve a final concentration of 100 nM, 1,000 nM, and 10,000 nM as well as no oligonucleotide diluent controls. After a treatment period of approximately one week, total RNA was isolated from the cells and Runx1 mRNA levels were measured by quantitative real-time PCR. Mouse Taqman Assay ID Mm01213404 was used to measure mRNA levels. Runx1 levels were compared to the housekeeping gene Beta Actin Mouse Taqman Assay ID Mm02619580_g1. Results are presented in Table 2 below as relative expression of Runx1, normalized to the no oligonucleotide control. For each of the ASOs listed in Table 2, each cytosine is a 5-methyl cytosine A value of 0% reduction indicates that the compound had no effect or increased RNA concentrations in the cell. As illustrated in the table below, Runx1 mRNA levels were reduced in a dose-dependent manner in modified oligonucleotide-treated cells.

### Example 3: Effect of MOE Gapmers with Internucleoside Linkages on Human RUNX1 In Vitro, Single Dose

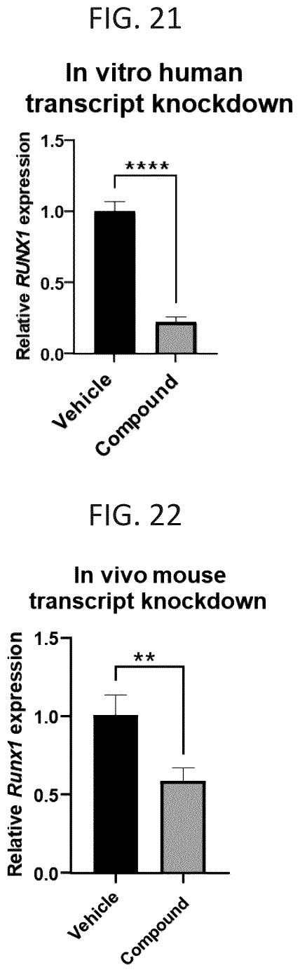

Summary: HMC-3s, an immortalized microglia-like cell line, were cultured in EMEM media. ASOs were introduced to cells via transfection utilizing a lipid-based transfection protocol. After 2 days of incubation, media was removed, cells were lysed, and RNA was extracted and analyzed via RT-qPCR with probes targeting RUNX1 and β-Actin as a control. Effect of an exemplary ASO on human transcript knockdown in vitro is provided in FIG. 21.

Modified oligonucleotides complementary to both a human and mouse RUNX1 nucleic acid were designed and tested for their effect on RUNX1 in vitro. The modified oligonucleotides were tested in a series of experiments that had similar culture conditions. Human immortalized microglia-like cells, referred to as I-IMC-3s, were plated at a concentration of 3,000 cells per well in 96 well plates and were transfected via lipid-based transfection to introduce modified oligonucleotides. Modified oligonucleotides were introduced to reach a final concentration of 1 ng/μL. After approximately 2 days, RNA was isolated from the cells and RUNX1 mRNA levels were measured by quantitative real-time PCR. Human Taqman Assay ID Hs01021970 ml was used to measure mRNA levels. RUNX1 levels were compared to the housekeeping gene Beta Actin Human Tagman Assay ID Is01060665_g1. Results are presented in Table 1 below as relative expression of RUNX1, normalized to the no oligonucleotide control. A value of 0% reduction indicates that the compound had no effect or increased RNA concentrations in the cell. The modified oligonucleotide in Table 3 is a 5-10-5 mixed MOE gapmer. The gapmers are 20 nucleotides in length, wherein the central gap segment comprises ten 2′-deoxynucleosides and is flanked on either side by segments of five 2′MOE nucleosides. The internucleoside linkages are phosphorothioate linkages.

The modified oligonucleotide listed in Table 3 below is complementary to human RUNX1 nucleic acid sequence SEQ ID NO: 10, as indicated. For the ASO listed in Table 3, each cytosine is a 5-methyl cytosine. A value of 0% reduction indicates that the compound had no effect or increased mRNA concentrations in the cell. As shown below, modified oligonucleotides complementary to RUNX1 reduced the amount of RUNX1 mRNA.

### Example 4: Potency of Modified Oligonucleotides Complementary to Mouse Runx1 in Mice

Summary: ASOs were reconstituted in sterile 0.9% NaCl. 4 μL of ASO or vehicle were slowly injected intracerebral ventricularly (ICV) into the right ventricle of sedated mice. At takedown, mice were perfused to remove peripheral immune cells from the brain. Brains were dissociated, and microglia were isolated. Isolated microglia were lysed, RNA was extracted and levels of Runx1 transcript were determined via RT-qPCR with probes targeting Runx1 and β-Actin as a control. Effect of an exemplary ASO on mouse transcript knockdown in vivo shown in FIG. 22.

Modified oligonucleotides described above were tested in a C57/B16J mouse.

Treatment: Mice were divided into groups of 4-7 mice each. Groups were given a single ICV bolus of oligonucleotide at a dose of 150 μg and sacrificed two or three weeks later. The PBS injected group served as the control group to which oligonucleotide groups were compared.

RNA Analysis: After two or three weeks, mice were sacrificed and microglia were extracted from the brain. RNA from microglia was extracted for real time PCR analysis of measurement of mRNA expression of Runx1 using Mouse Taqman Assay ID Mm01213404 and compared to the housekeeping gene Beta Actin Mouse Tagman Assay ID Mm02619580_g1. Results are presented in Table 4 below as percent change of mRNA, relative to PBS control.

The modified oligonucleotide in Table 4 below is complementary to human RUNX1 nucleic acid sequence SEQ ID NO: 15, as indicated. For the ASO listed in Table 4, each cytosine is a 5-methyl cytosine. As shown in Table 4 below, treatment with modified oligonucleotides resulted in significant reduction of Runx1 in comparison to the PBS control. Results are a combination of three individual studies.

TABLE 1

| | | ASO | | |
|---|---|---|---|---|
| | | Sequence | | |
| | Tar- | 5′ to 3′ | | |
| | geted | (X is 5- | Tar- | 2′MOE |
| ASO | Se- | methyl | geted | knock- |
| Name | quence | cytosine) | Exon | down |
| Compound | SEQ ID | GGGGTTGAGX | 8 | |
| 1/SEQ ID | NO: 1 | AGXGXGGAGX | | |
| NO: 20 | | | | |
| Compound | SEQ ID | TXGXTXTGGT | 8 | |
| 2/SEQ ID | NO: 2 | TXGGGAGGXT | | |
| NO: 21 | | | | |
| Compound | SEQ ID | GTGATTTTGA | 5 | 72.6 |
| 3/ SEQ ID | NO: 3 | TGGXTXTGTG | | |
| NO: 22 | | | | |
| Compound | SEQ ID | XTGTGATTTT | 5 | 53.0 |
| 4/ SEQ ID | NO: 4 | GATGGXTXTG | | |
| NO: 23 | | | | |
| Compound | SEQ ID | AXTGTGATTT | 5 | |
| 5/ SEQ ID | NO: 5 | TGATGGXTXT | | |
| NO: 24 | | | | |
| Compound | SEQ ID | XAXTGTGATT | 5 | |
| 6/ SEQ ID | NO: 6 | TTGATGGXTX | | |
| NO: 25 | | | | |
| Compound | SEQ ID | XXAXTGTGAT | 5 | |
| 7/ SEQ ID | NO: 7 | TTTGATGGXT | | |
| NO: 26 | | | | |
| Compound | SEQ ID | TXXAXTGTGA | 5 | |
| 8/ SEQ ID | NO: 8 | TTTTGATGGX | | |
| NO: 27 | | | | |
| Compound | SEQ ID | GTGATGGTXA | 5 | |
| 9/ SEQ ID | NO: 9 | GAGTGAAGXT | | |
| NO: 28 | | | | |
| Compound | SEQ ID | GTGAXXAGAG | 4 | 61.2 |
| 10/ SEQ | NO: 10 | TGXXATXTGG | | |
| ID NO: 29 | | | | |
| Compound | SEQ ID | GAGTAGTTTT | 4 | |
| 11/ SEQ | NO: 11 | XATXATTGXX | | |
| ID NO: 30 | | | | |
| Compound | SEQ ID | AGXGATGGGX | 3 | |
| 12/ SEQ | NO: 12 | AGGGTXTTGT | | |
| ID NO: 31 | | | | |
| Compound | SEQ ID | XTGTGATTTT | 5 | 64.1 |
| 13/ SEQ | NO: 13 | GATGGXTXTA | | |
| ID NO: 32 | | | | |
| Compound | SEQ ID | GTGAXXAGAG | 4 | 76.0 |
| 14/ SEQ | NO: 14 | TGXXATXXGG | | |
| ID NO: 33 | | | | |
| Compound | SEQ ID | AXAGTGAXXA | 4 | 71.9 |
| 15/ SEQ | NO: 15 | GAGTGXXATX | | |
| ID NO: 34 | | | | |
| Compound | SEQ ID | XXGAGTAGTT | 4 | 55.4 |
| 16/ SEQ | NO: 16 | TTXATXATTG | | |
| ID NO: 35 | | | | |
| Compound | SEQ ID | AXXTGGTTXT | 4 | 12.6 |
| 17/ SEQ | NO: 17 | TXATGGXTGX | | |
| ID NO: 36 | | | | |
| Compound | SEQ ID | GAXAGTGATG | 5 | 30.7 |
| 18/ SEQ | NO: 18 | GTXAGAGTGA | | |
| ID NO: 37 | | | | |
| Compound | SEQ ID | AXGGTGAXXA | 4 | 58.5 |
| 19/ SEQ | NO: 19 | GAGTGXXATX | | |
| ID NO: 38 | | | | |

TABLE 2

| | | ASO | | | | |
|---|---|---|---|---|---|---|
| | | Sequence | | | | |
| | Tar- | 5′ to 3′ | | | | |
| | geted | (X is 5- | Tar- | | | |
| ASO | Se- | methyl | geted | 10 | 1 | 0.1 |
| Name | quence | cytosine) | Exon | μM | μM | μM |
| Com- | SEQ ID | GTGATTTTGA | 5 | 86.0 | 76.3 | 20.7 |
| pound | NO: 3 | TGGXTXTGTG | | | | |
| 3/SEQ | | | | | | |
| ID | | | | | | |
| NO: | | | | | | |
| 22 | | | | | | |
| Com- | SEQ ID | GTGAXXAGAG | 3 | 85.0 | 52.0 | 6.6 |
| pound | NO: 10 | TGXXATXTGG | | | | |
| 10/ | | | | | | |
| SEQ | | | | | | |
| ID | | | | | | |
| NO: | | | | | | |
| 29 | | | | | | |
| Com- | SEQ ID | GTGAXXAGAG | 4 | 61.0 | 60.0 | 19 |
| pound | NO: 14 | TGXXATXXGG | | | | |
| 14/ | | | | | | |
| SEQ | | | | | | |
| ID | | | | | | |
| NO: | | | | | | |
| 33 | | | | | | |
| Com- | SEQ ID | AXAGTGAXXA | 4 | 74.6 | | 12.8 |
| pound | NO: 15 | GAGTGXXATX | | | | |
| 15/ | | | | | | |
| SEQ | | | | | | |
| ID | | | | | | |
| NO: | | | | | | |
| 34 | | | | | | |

TABLE 3

| | | ASO | | |
|---|---|---|---|---|
| | | Sequence | | |
| | Tar- | 5′ to 3′ | | |
| | geted | (X is 5- | Tar- | |
| ASO | Se- | methyl | geted | knock- |
| Name | quence | cytosine) | Exon | down |
| Com- | SEQ ID | GTGAXXAGAG | 4 | 77.6 |
| pound | NO: 10 | TGXXATXTGG | | |
| 10/ | | | | |
| SEQ | | | | |
| ID | | | | |
| NO: | | | | |
| 29 | | | | |

TABLE 4

| | | | | knock- | knock- |
|---|---|---|---|---|---|
| | | ASO | | down | down |
| | | Sequence | | 14 | 21 |
| | Tar- | 5′ to 3′ | | days | days |
| | geted | (X is 5- | Tar- | after | after |
| ASO | Se- | methyl | geted | injec- | injec- |
| Name | quence | cytosine) | Exon | tion | tion |
| Compound | SEQ ID | AXAGTGAXX | 4 | 22 | 41 |
| 15/SEQ ID | NO: 15 | AGAGTGXXA | | | |
| NO: 34 | | TX | | | |

The sequences of SEQ ID NO: 1-SEQ ID) NO: 19 listed in Tables 1-4 are provided in Table

TABLE 5

| SEQ ID NO | RNA SEQUENCE |
|---|---|
| SEQ ID NO: 1 | GCUCCGCGCUGCUCAACCCC |
| SEQ ID NO: 2 | AGCCUCCCGAACCAGAGCGA |
| SEQ ID NO: 3 | CACAGAGCCAUCAAAAUCAC |
| SEQ ID NO: 4 | CAGAGCCAUCAAAAUCACAG |
| SEQ ID NO: 5 | AGAGCCAUCAAAAUCACAGU |
| SEQ ID NO: 6 | GAGCCAUCAAAAUCACAGUG |
| SEQ ID NO: 7 | AGCCAUCAAAAUCACAGUGG |
| SEQ ID NO: 8 | GCCAUCAAAAUCACAGUGGA |
| SEQ ID NO: 9 | AGCUUCACUCUGACCAUCAC |
| SEQ ID NO: 10 | CCAGAUGGCACUCUGGUCAC |
| SEQ ID NO: 11 | GGCAAUGAUGAAAACUACUC |
| SEQ ID NO: 12 | ACAAGACCCUGCCCAUCGCU |
| SEQ ID NO: 13 | UAGAGCCAUCAAAAUCACAG |
| SEQ ID NO: 14 | CCGGAUGGCACUCUGGUCAC |
| SEQ ID NO: 15 | GAUGGCACUCUGGUCACUGU |
| SEQ ID NO: 16 | CAAUGAUGAAAACUACUCGG |
| SEQ ID NO: 17 | GCAGCCAUGAAGAACCAGGU |
| SEQ ID NO: 18 | UCACUCUGACCAUCACUGUC |
| SEQ ID NO: 19 | GAUGGCACUCUGGUCACCGU |

The sequences of SEQ ID NO: 20-SEQ ID NO: 38 listed in Tables 1-4 are provided in Table 6 below.

TABLE 6

| | SEQUENCE 5′ to 3′ |
|---|---|
| | (X is 5-methyl |
| SEQ ID NO | cytosine) |
| SEQ ID NO: 20 | GGGGTTGAGXAGXGXGGAGX |
| SEQ ID NO: 21 | TXGXTXTGGTTXGGGAGGXT |
| SEQ ID NO: 22 | GTGATTTTGATGGXTXTGTG |
| SEQ ID NO: 23 | XTGTGATTTTGATGGXTXTG |
| SEQ ID NO: 24 | AXTGTGATTTTGATGGXTXT |
| SEQ ID NO: 25 | XAXTGTGATTTTGATGGXTX |
| SEQ ID NO: 26 | XXAXTGTGATTTTGATGGXT |
| SEQ ID NO: 27 | TXXAXTGTGATTTTGATGGX |
| SEQ ID NO: 28 | GTGATGGTXAGAGTGAAGXT |
| SEQ ID NO: 29 | GTGAXXAGAGTGXXATXTGG |
| SEQ ID NO: 30 | GAGTAGTTTTXATXATTGXX |
| SEQ ID NO: 31 | AGXGATGGGXAGGGTXTTGT |
| SEQ ID NO: 32 | XTGTGATTTTGATGGXTXTA |
| SEQ ID NO: 33 | GTGAXXAGAGTGXXATXXGG |
| SEQ ID NO: 34 | AXAGTGAXXAGAGTGXXATX |
| SEQ ID NO: 35 | XXGAGTAGTTTTXATXATTG |
| SEQ ID NO: 36 | AXXTGGTTXTTXATGGXTGX |
| SEQ ID NO: 37 | GAXAGTGATGGTXAGAGTGA |
| SEQ ID NO: 38 | AXGGTGAXXAGAGTGXXATX |

The sequences of SEQ ID NO: 39-SEQ ID NO: 57 listed in Table 7 below is the DNA Sequence targeted by ASO compounds disclosed herein:

TABLE 7

| SEQ ID NO | DNA SEQUENCE |
|---|---|
| SEQ ID NO: 39 | GCTCCGCGCTGCTCAACCCC |
| SEQ ID NO: 40 | AGCCTCCCGAACCAGAGCGA |
| SEQ ID NO: 41 | CACAGAGCCATCAAAATCAC |
| SEQ ID NO: 42 | CAGAGCCATCAAAATCACAG |
| SEQ ID NO: 43 | AGAGCCATCAAAATCACAGT |
| SEQ ID NO: 44 | GAGCCATCAAAATCACAGTG |
| SEQ ID NO: 45 | AGCCATCAAAATCACAGTGG |
| SEQ ID NO: 46 | GCCATCAAAATCACAGTGGA |
| SEQ ID NO: 47 | AGCTTCACTCTGACCATCAC |
| SEQ ID NO: 48 | CCAGATGGCACTCTGGTCAC |
| SEQ ID NO: 49 | GGCAATGATGAAAACTACTC |
| SEQ ID NO: 50 | ACAAGACCCTGCCCATCGCT |
| SEQ ID NO: 51 | TAGAGCCATCAAAATCACAG |
| SEQ ID NO: 52 | CCGGATGGCACTCTGGTCAC |
| SEQ ID NO: 53 | GATGGCACTCTGGTCACTGT |
| SEQ ID NO: 54 | CAATGATGAAAACTACTCGG |
| SEQ ID NO: 55 | GCAGCCATGAAGAACCAGGT |
| SEQ ID NO: 56 | TCACTCTGACCATCACTGTC |
| SEQ ID NO: 57 | GATGGCACTCTGGTCACCGT |

### Example 5: Effect of MOE Gapmers with Internucleoside Linkages on Mouse Runx1 in Mouse Microglia by Free Uptake, Single Dose

RAJI cells, immortalized from B-lymphocytes, were cultured in standard culture conditions. Modified oligonucleotides were added directly to media at a 2 μM concentration in the media and cells were transfected using electroporation. After approximately 24 hours, a one-step RT-qPCR reaction was utilized to extract RNA, synthesize cDNA and quantify expression levels with probes targeting RUNX1 and β-Actin as a control, Results are presented in Table 8 below.

Each modified oligonucleotide listed in Table 8 below is complementary to human RUNX1 nucleic acid sequences SEQ ID NO: 1-SEQ ID NO: 19, as indicated. For each of the ASOs listed in Table 8, each cytosine is a 5-methyl cytosine.

TABLE 8

| | Targeted | Targeted | 2′MOE |
|---|---|---|---|
| ASO Name | Sequence | Exon | knockdown |
| Compound | SEQ ID | 8 | 28 |
| 1/SEQ ID | NO: 1 | | |
| NO: 20 | | | |
| Compound | SEQ ID | 8 | 33.33 |
| 2/SEQ ID | NO: 2 | | |
| NO: 21 | | | |
| Compound | SEQ ID | 5 | 82.4 |
| 3/SEQ ID | NO: 3 | | |
| NO: 22 | | | |
| Compound | SEQ ID | 5 | 86.42 |
| 4/SEQ ID | NO: 4 | | |
| NO: 23 | | | |
| Compound | SEQ ID | 5 | 79.03 |
| 5/SEQ ID | NO: 5 | | |
| NO: 24 | | | |
| Compound | SEQ ID | 5 | 67.85 |
| 6/SEQ ID | NO: 6 | | |
| NO: 25 | | | |
| Compound | SEQ ID | 5 | 64.69 |
| 7/SEQ ID | NO: 7 | | |
| NO: 26 | | | |
| Compound | SEQ ID | 5 | 65.86 |
| 8/SEQ ID | NO: 8 | | |
| NO: 27 | | | |
| Compound | SEQ ID | 5 | 11.67 |
| 9/SEQ ID | NO: 9 | | |
| NO: 28 | | | |
| Compound | SEQ ID | 4 | −43.1 |
| 10/SEQ ID | NO: 10 | | |
| NO: 29 | | | |
| Compound | SEQ ID | 4 | −41.5 |
| 11/SEQ ID | NO: 11 | | |
| NO: 30 | | | |
| Compound | SEQ ID | 3 | 8.67 |
| 12/SEQ ID | NO: 12 | | |
| NO: 31 | | | |
| Compound | SEQ ID | 5 | 84.57 |
| 13/SEQ ID | NO: 13 | | |
| NO: 32 | | | |
| Compound | SEQ ID | 4 | 31.78 |
| 14/SEQ ID | NO: 14 | | |
| NO: 33 | | | |
| Compound | SEQ ID | 4 | −25.9 |
| 15/SEQ ID | NO: 15 | | |
| NO: 34 | | | |
| Compound | SEQ ID | 4 | 4.64 |
| 16/SEQ ID | NO: 16 | | |
| NO: 35 | | | |
| Compound | SEQ ID | 4 | 0.15 |
| 17/SEQ ID | NO: 17 | | |
| NO: 36 | | | |
| Compound | SEQ ID | 5 | 38.66 |
| 18/SEQ ID | NO: 18 | | |
| NO: 37 | | | |
| Compound | SEQ ID | 4 | 34.11 |
| 19/SEQ ID | NO: 19 | | |
| NO: 38 | | | |

### Example 6: Suppression of Microglial Production of Pro-Inflammatory Cytokines

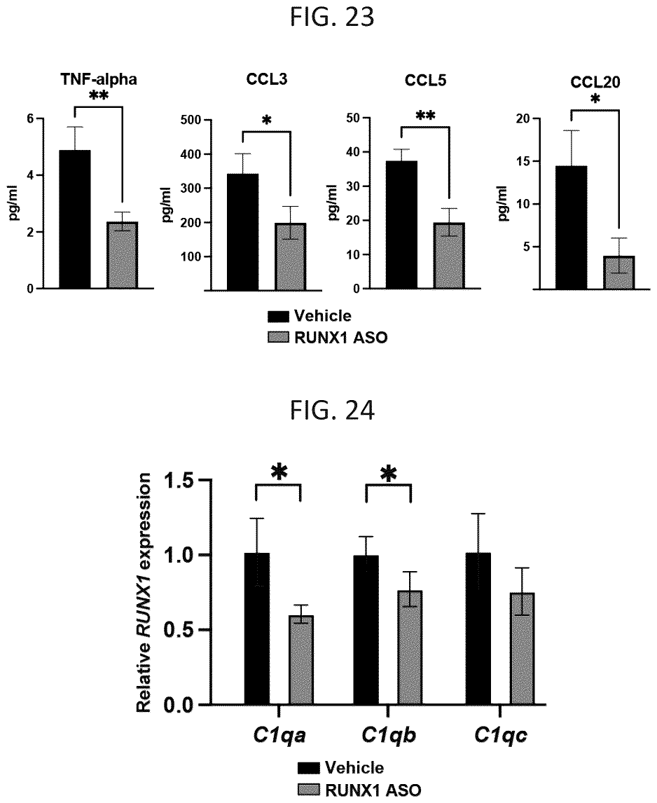

Microglia were extracted from the cortices of 8-day old mice and were cultured in vitro for 3 days. After 3 days, ASOs were added to the media to obtain a final concentration of 10 PM in the media. ASOs were left on the cells for 7 days. Media was replaced with fresh media for 3 days at which point media, was removed and profiled via cytokine array. Effect of an exemplary ASO on TNF-alpha, CCL3, CCL5, and CCL20 in comparison to vehicle is shown in FIG. 23.

### Example 7: Suppression of Microglial Production of Complement Related Genes

Microglia were extracted from the cortices of 8-day old mice and were cultured in vitro for 3 days. After 3 days, ASOs were added to the media to obtain a final concentration of 10 uM in the media. ASOs were left on the cells for 7 days. Media was removed, cells were lysed, and RNA was extracted. Levels of RNA were determined using RT-qPCR with probes against C1qa, C1qb, and C1qc and control probes against β-Actin. Effect of an exemplary ASO on C1qa, C1qb, and C1qc in comparison to vehicle is shown in FIG. 24.

While preferred embodiments of the present disclosure have been shown and described herein, it will be understood to those skilled in the art that such embodiments are provided by way of example only. It is not intended that the embodiments be limited by the specific examples provided within the specification. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

We claim:

1. An oligomeric compound, or a salt thereof, comprising a modified oligonucleotide consisting of 10-30 linked nucleosides wherein the modified oligonucleotide is at least 90% complementary to any of the nucleobase sequences of SEQ ID NO: 1-19, when measured across the entire nucleobase sequence of the modified oligonucleotide, and wherein the modified oligonucleotide comprises at least one modification selected from a modified sugar, a sugar surrogate, and a modified internucleoside linkage.

2. An oligomeric compound, or a salt thereof, comprising a modified oligonucleotide consisting of 10-30 linked nucleosides wherein the modified oligonucleotide has a nucleobase sequence that is targeted to any of the nucleobase sequences of SEQ ID NO: 39-57, when measured across the entire nucleobase sequence of the modified oligonucleotide, and wherein the modified oligonucleotide comprises at least one modification selected from a modified sugar, a sugar surrogate, and a modified internucleoside linkage.

3. The oligomeric compound of claim 2, wherein the modified oligonucleotide comprises at least two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31 or 32 modifications, or number of modifications within a range defined by any of the preceding numbers, selected from a modified sugar, a sugar surrogate, and a modified internucleoside linkage.

4. The oligomeric compound of claim 2, wherein the modified oligonucleotide is at least 90% complementary to any of the nucleobase sequences of SEQ ID NO: 39-57.

5. The oligomeric compound of claim 2, wherein the modified oligonucleotide is 100% complementary to any of the nucleobase sequences of SEQ ID NO: 39-57.

6. An oligomeric compound (formula ASO (15)) according to the following formula: 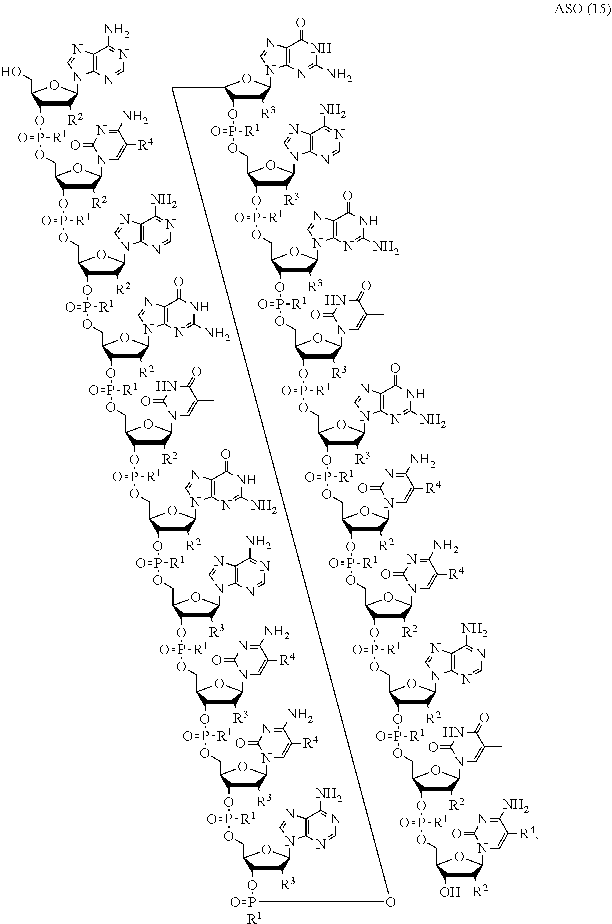
or a salt thereof, wherein:
each R¹ is independently selected from the group consisting of OH and SH;
each R² is independently selected from the group consisting of hydrogen, halo, OCH₃, OCF₃, OCH₂CH₃, OCH₂CH₂OCH₃, and OCH₂CF₃;
each R³ is independently selected from the group consisting of hydrogen, halo, OH, OCH₃, OCF₃, OCH₂CH₃, OCH₂CH₂OCH₃, and OCH₂CF₃; and
each R⁴ is independently selected from the group consisting of hydrogen and CH₃.

7. The compound of claim 6, wherein R³ is hydrogen.

8. The compound of claim 6, wherein each R² is OCH₂CH₃ or OCH₃.

9. The compound of claim 6, wherein each R² is OCH₂CH₂OCH₃.

10. The compound of claim 6, wherein each R¹ is SH.

11. A pharmaceutical composition comprising an oligomeric compound of claim 1, or a salt thereof, and a pharmaceutically acceptable carrier or diluent.

12. A method of improving cognitive function in a subject in need thereof, comprising administering a compound of claim 1, or a salt thereof to the subject.

13. The method of claim 12, wherein the subject has an overexpression of RUNX1.

14. The method of claim 12, wherein the subject has Down syndrome.

15. The method of claim 12, wherein the subject has Alzheimer's disease.

16. A method of treating a disease associated with RUNX1 in a subject in need thereof, comprising administering a compound of claim 1, or a salt thereof to the subject.

17. A method of treating cognitive loss associated with a neurodegenerative disease or disorder, the method comprising:
identifying a subject in need of treatment for cognitive decline; and
administering a therapeutically effective amount of a RUNX1 inhibitor to the subject thereby reducing the expression of RUNX1 in microglia of the subject and treating the cognitive loss in the subject.

18. The method of claim 17, wherein the subject is diagnosed with a neurodegenerative disease or disorder, wherein the neurodegenerative disease or disorder comprises Alzheimer's disease, Parkinson's disease, Down syndrome, Down syndrome associated dementia, prion disease, Amyotrophic lateral sclerosis, Nasu Hakola disorder, spinal muscular atrophy, or spinocerebellar ataxia.

19. The method of claim 18, wherein the neurodegenerative disease or disorder is Down syndrome or Down syndrome associated dementia.

20. The method of claim 18, wherein the neurodegenerative disease or disorder is Alzheimer's disease.
